(19) United States
(12) Patent Application Publication
(10) Pub. No.: US 2023/0173077 A1
(43) Pub. Date: Jun. 8, 2023

(54) FATTY ACYL AND FATTY ETHER CONJUGATES OF REMDESIVIR AND ITS ACTIVE METABOLITES AS ANTIVIRALS

(71) Applicant: AJK BIOPHARMACEUTICAL, LLC, Irvine, CA (US)

(72) Inventors: Keykavous PARANG, Irvine, CA (US); Rakesh TIWARI, Irvine, CA (US); Naglaa Salem EL-SAYED, Irvine, CA (US)

(21) Appl. No.: 17/922,576

(22) PCT Filed: Apr. 30, 2021
(86) PCT No.: PCT/US21/30265

§ 371 Date: Oct. 31, 2022

Related U.S. Application Data
(60) Provisional application No. 63/017,729, filed on Apr. 30, 2020.
(60) Provisional application No. 63/053,349, filed on Jul. 17, 2020.

(51) Int. Cl. A61K 47/54; A61K 45/06

(52) U.S. Cl. CPC A61K 47/542; A61K 45/06

(57) ABSTRACT

Antiviral compounds, pharmaceutical compositions containing the same, and methods of treating viral infections or medical disorders resulting from viral infections are disclosed.

### CROSS-REFERENCE TO RELATED APPLICATIONS

This application claims the benefit of priority to U.S. Provisional Application No. 63/017,729, filed Apr. 30, 2020, and U.S. Provisional Application No. 63/053,349, filed Jul. 17, 2020, the disclosures of which are incorporated herein by reference in their entirety.

### TECHNICAL FIELD

This disclosure relates to antiviral compounds and uses thereof, and more particularly to derivatives of remdesivir, its precursors, and its active metabolites for treatment of coronavirus infections.

### BACKGROUND

The background description includes information that may be useful in understanding the present invention. It is not an admission that any of the information provided herein is prior art or relevant to the presently claimed invention, or that any publication specifically or implicitly referenced is prior art.

The increasing prevalence of COVID-19 is a serious public health problem affecting people globally. COVID-19 is taking a devastating toll on human lives. The latest human mortality of COVID-19 pandemic infection is 3,162,697 people, while 127,725,261 people have been infected globally on Apr. 283, 20210. The discovery and approval of new compounds take several years. Therefore, several existing drug and potential drug candidates such as remdesivir and other antiviral agents have been considered to be repurposed as COVID-19 treatments.

Remdesivir (GS-5734, FIG. 1) was developed by Gilead and found to be effective against severe acute respiratory syndrome (SARS) and middle east respiratory syndrome (MERS) in animal models [1, 2]. All publications herein are incorporated by reference to the same extent as if each individual publication or patent application were specifically and individually indicated to be incorporated by reference. Where a definition or use of a term in an incorporated reference is inconsistent or contrary to the definition of that term provided herein, the definition of that term provided herein applies and the definition of that term in the reference does not apply. Remdesivir was also evaluated by Gilead for SARS-CoV-2 in early 2020. It was then used by Chinese medical researchers in patients for clinical testing in late January 2020, suggesting favorable inhibitory on SARS-CoV-2 (unpublished results). Since then, several clinical trials of remdesivir have been initiated by China and the World Health Organization (WHO). Furthermore, randomized clinical trials are ongoing or planned to determine the effect in the improvement in patients recovery [3]. Remdesivir has been approved or authorized for emergency use for the treatment of COVID-19 around the world (U.S. Food and Drug Administration Approves Gilead's Antiviral Veklury (remdesivir) for Treatment of COVID-19”. Gilead Sciences, Inc. 22 Oct. 2020).

Remdesivir is a phosphoramidite prodrug of an adenine C-nucleoside; it has a short plasma half-life (0.39 h); and is used as the IV dosage form. The compound is a broad-spectrum antiviral nucleotide prodrug that is metabolized first to the active triphosphate analog that inhibits RNA-dependent RNA polymerase (RdRp), resulting in diminished viral RNA replication [4].

Remdesivir exhibited EC₅₀ and TC₅₀ values of 0.07 μM and >2.0, respectively, against HCoV-229E. Meanwhile, 5′-O-tetradecanoyl ester conjugate of 2′,3′-dideoxy-5-fluoro-3′-thiacytidine (emtricitabine, FTC) demonstrated an EC₅₀ value of 82 μM. Clinical efficacy against SARS-CoV-2 and other coronaviruses, however, is not yet firmly established. In addition, the development of resistance to such single-mechanism antiviral drugs over time is highly likely.

Inventors have previously shown that the conjugation of certain fatty acids to the anti-HIV nucleoside reverse transcriptase inhibitors NRTIs, such as 3′-fluoro-3′-deoxythymidine (FLT, alovudine) [5-7], 2′,3′-dideoxy-3′-thiacytidine (lamivudine, 3TC), and 2′,3′-dideoxy-5-fluoro-3′-thiacytidine (emtricitabine, FTC) [8-11], enhanced activity against X4, R5, cell-associated, and/or multi-drug resistant virus when compared with their parent nucleosides. This, however, is not predictive of efficacy with SARS-CoV-2 and other coronaviruses.

Thus, there is still a need for safe and effective antiviral compounds that are effective against SARS-CoV-2 and other coronaviruses while also decreasing the likelihood of the development of resistant mutations

### SUMMARY

In accordance with the purposes of the disclosed compositions and methods, as embodied and broadly described herein, the disclosed subject matter relates to antiviral compounds and methods of making and using the same. In particular, the disclosed subject matter relates to compounds of Formula I, II, I-a, and II-a as described herein, or pharmaceutically acceptable salts thereof. Also described are pharmaceutical compositions comprising the compounds described herein and a pharmaceutically acceptable carrier or excipient. Also described are methods of treating infections with a virus (such as a coronavirus) using the compounds or compositions described herein. Also disclosed are methods of treating medical disorders resulting from an infection with a virus using the compounds or compositions described herein.

Additional advantages of the disclosed compounds, compositions, and methods will be set forth in part in the description which follows, and in part will be obvious from the description, or can be learned by practice of the disclosed compounds, compositions, and methods. The advantages of the disclosed compounds, compositions, and methods will be realized and attained by means of the elements and combinations particularly pointed out in the appended claims. It is to be understood that both the foregoing general description and the following detailed description are exemplary and explanatory only and are not restrictive of the disclosed compounds, compositions, and methods, as claimed.

### DESCRIPTION OF DRAWINGS

The accompanying figures, which are incorporated in and constitute a part of this specification, illustrate several aspects and together with the description, serve to explain the principles of the invention.

FIG. 1 shows the chemical structure of remdesivir (1) and GS 441524 (2).

FIG. 2 shows the general structure of compounds in Formulas I and II.

FIG. 3 demonstrates the synthesis of mono fatty acyl substituted and di fatty acyl substituted derivatives of remdesivir as a representative compounds in Formula I.

FIG. 4 shows the synthesis amino substituted derivatives of remdesivir as a representative compounds in Formula I.

FIG. 5 demonstrates the synthesis of mono fatty acyl substituted derivatives of compounds in Formula II at 5′-position or amino group.

FIG. 6 demonstrates the synthesis of monofatty acyl substituted derivatives of compounds in Formula II at 2′ or 3′ positions.

FIG. 7 demonstrate the structures of fatty ether derivatives of compounds in Formulas I and II.

FIG. 8 demonstrates the synthesis of mono fatty ether substituted and di fatty ether substituted derivatives of remdesivir as representative compounds in Formula I and II.

FIG. 9 schematically depicts an example of a direct synthesis of fatty acyl conjugates of remdesivir using unprotected remdesivir that are purified by HPLC as representative compounds in Formula I.

FIG. 10 shows chemical structures of representative mono fatty acyl conjugates of remdesivir in Formula I.

FIG. 11 shows the chemical structures of representative di fatty acyl conjugates of remdesivir in Formula I.

FIG. 12 shows the synthesis of selected amino substituted derivatives of remdesivir as a representative compounds in Formula I.

FIG. 13 shows the representative amino substituted fatty acyl conjugate of remdesivir in Formula I.

FIG. 14 demonstrates the synthesis of representative mono fatty acyl conjugates of remdesivir in Formula I.

FIG. 15 shows the ¹H NMR of compound 3′a.

FIG. 16 shows the ¹³C NMR of compound 3′a.

FIG. 17 shows the ¹H NMR of compound Yb.

FIG. 18 shows the ¹³C NMR of compound Yb.

FIG. 19 shows the ¹H NMR of compound 3′c.

FIG. 20 shows the ¹³C NMR of compound 3′c.

### DETAILED DESCRIPTION

The following description of the disclosure is provided as an enabling teaching of the disclosure in its best, currently known embodiments. Many modifications and other embodiments disclosed herein will come to mind to one skilled in the art to which the disclosed compositions and methods pertain having the benefit of the teachings presented in the foregoing descriptions and the associated drawings. Therefore, it is to be understood that the disclosures are not to be limited to the specific embodiments disclosed and that modifications and other embodiments are intended to be included within the scope of the appended claims. The skilled artisan will recognize many variants and adaptations of the aspects described herein. These variants and adaptations are intended to be included in the teachings of this disclosure and to be encompassed by the claims herein.

Although specific terms are employed herein, they are used in a generic and descriptive sense only and not for purposes of limitation.

As can be apparent to those of skill in the art upon reading this disclosure, each of the individual embodiments described and illustrated herein has discrete components and features which may be readily separated from or combined with the features of any of the other several embodiments without departing from the scope or spirit of the present disclosure.

Any recited method can be carried out in the order of events recited or in any other order that is logically possible. That is, unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which can require independent confirmation.

It is also to be understood that the terminology used herein is for the purpose of describing particular aspects only and is not intended to be limiting. Unless defined otherwise, all technical and scientific terms used herein have the same meaning as commonly understood by one of ordinary skill in the art to which the disclosed compositions and methods belong. It can be further understood that terms, such as those defined in commonly used dictionaries, should be interpreted as having a meaning that is consistent with their meaning in the context of the specification and relevant art and should not be interpreted in an idealized or overly formal sense unless expressly defined herein.

Prior to describing the various aspects of the present disclosure, the following definitions are provided and should be used unless otherwise indicated. Additional terms may be defined elsewhere in the present disclosure.

### Definitions

As used herein, “comprising” is to be interpreted as specifying the presence of the stated features, integers, steps, or components as referred to, but does not preclude the presence or addition of one or more features, integers, steps, or components, or groups thereof. Moreover, each of the terms “by”, “comprising,” “comprises”, “comprised of,” “including,” “includes,” “included,” “involving,” “involves,” “involved,” and “such as” are used in their open, non-limiting sense and may be used interchangeably. Further, the term “comprising” is intended to include examples and aspects encompassed by the terms “consisting essentially of” and “consisting of.” Similarly, the term “consisting essentially of” is intended to include examples encompassed by the term “consisting of.

As used in the specification and the appended claims, the singular forms “a,” “an” and “the” include plural referents unless the context clearly dictates otherwise. Thus, for example, reference to “a compound”, “a virus”, or “a carrier”, includes, but is not limited to, two or more such compounds, viruses, or carriers, and the like.

It should be noted that ratios, concentrations, amounts, and other numerical data can be expressed herein in a range format. It can be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about,” it can be understood that the particular value forms a further aspect. For example, if the value “about 10” is disclosed, then “10” is also disclosed.

When a range is expressed, a further aspect includes from the one particular value and/or to the other particular value. For example, where the stated range includes one or both of the limits, ranges excluding either or both of those included limits are also included in the disclosure, e.g. the phrase “x to y” includes the range from ‘x’ to ‘y’ as well as the range greater than ‘x’ and less than ‘y’. The range can also be expressed as an upper limit, e.g. ‘about x, y, z, or less’ and should be interpreted to include the specific ranges of ‘about x’, ‘about y’, and ‘about z’ as well as the ranges of ‘less than x’, less than y’, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y’, and ‘greater than z’. In addition, the phrase “about ‘x’ to ‘y’”, where ‘x’ and ‘y’ are numerical values, includes “about ‘x’ to about ‘y’”.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

As used herein, the terms “about,” “approximate,” “at or about,” and “substantially” mean that the amount or value in question can be the exact value or a value that provides equivalent results or effects as recited in the claims or taught herein. That is, it is understood that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In such cases, it is generally understood, as used herein, that “about” and “at or about” mean the nominal value indicated ±10% variation unless otherwise indicated or inferred. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

As used herein, the term “therapeutically effective amount” refers to an amount that is sufficient to achieve the desired therapeutic result or to have an effect on undesired symptoms but is generally insufficient to cause adverse side effects. The specific therapeutically effective dose level for any particular patient will depend upon a variety of factors including the disorder being treated and the severity of the disorder; the specific composition employed; the age, body weight, general health, sex and diet of the patient; the time of administration; the route of administration; the rate of excretion of the specific compound employed; the duration of the treatment; drugs used in combination or coincidental with the specific compound employed and like factors within the knowledge and expertise of the health practitioner and which may be well known in the medical arts. In the case of treating a particular disease or condition, in some instances, the desired response can be inhibiting the progression of the disease or condition. This may involve only slowing the progression of the disease temporarily. However, in other instances, it may be desirable to halt the progression of the disease permanently. This can be monitored by routine diagnostic methods known to one of ordinary skill in the art for any particular disease. The desired response to treatment of the disease or condition also can be delaying the onset or even preventing the onset of the disease or condition.

For example, it is well within the skill of the art to start doses of a compound at levels lower than those required to achieve the desired therapeutic effect and to gradually increase the dosage until the desired effect is achieved. If desired, the effective daily dose can be divided into multiple doses for purposes of administration. Consequently, single dose compositions can contain such amounts or submultiples thereof to make up the daily dose. The dosage can be adjusted by the individual physician in the event of any contraindications. It is generally preferred that a maximum dose of the pharmacological agents of the invention (alone or in combination with other therapeutic agents) be used, that is, the highest safe dose according to sound medical judgment. It will be understood by those of ordinary skill in the art however, that a patient may insist upon a lower dose or tolerable dose for medical reasons, psychological reasons or for virtually any other reasons.

A response to a therapeutically effective dose of a disclosed compound or pharmaceutical composition can be measured by determining the physiological effects of the treatment or medication, such as the decrease or lack of disease symptoms following administration of the treatment or pharmacological agent. Other assays will be known to one of ordinary skill in the art and can be employed for measuring the level of the response. The amount of a treatment may be varied for example by increasing or decreasing the amount of a disclosed compound and/or pharmaceutical composition, by changing the disclosed compound and/or pharmaceutical composition administered, by changing the route of administration, by changing the dosage timing and so on. Dosage can vary, and can be administered in one or more dose administrations daily, for one or several days. Guidance can be found in the literature for appropriate dosages for given classes of pharmaceutical products.

As used herein, the term “prophylactically effective amount” refers to an amount effective for preventing onset or initiation of a disease or condition.

As used herein, the term “prevent” or “preventing” refers to precluding, averting, obviating, forestalling, stopping, or hindering something from happening, especially by advance action. It is understood that where reduce, inhibit or prevent are used herein, unless specifically indicated otherwise, the use of the other two words is also expressly disclosed.

As used herein, the terms “optional” or “optionally” means that the subsequently described event or circumstance can or cannot occur, and that the description includes instances where said event or circumstance occurs and instances where it does not.

As used interchangeably herein, “subject,” “individual,” or “patient” can refer to a vertebrate organism, such as a mammal (e.g. human). “Subject” can also refer to a cell, a population of cells, a tissue, an organ, or an organism, preferably to human and constituents thereof.

As used herein, the terms “treating” and “treatment” can refer generally to obtaining a desired pharmacological and/or physiological effect. The effect can be, but does not necessarily have to be, prophylactic in terms of preventing or partially preventing a disease, symptom or condition thereof, such as an ophthalmological disorder. The effect can be therapeutic in terms of a partial or complete cure of a disease, condition, symptom or adverse effect attributed to the disease, disorder, or condition. The term “treatment” as used herein can include any treatment of ophthalmological disorder in a subject, particularly a human and can include any one or more of the following: (a) preventing the disease from occurring in a subject which may be predisposed to the disease but has not yet been diagnosed as having it; (b) inhibiting the disease, i.e., arresting its development; and (c) relieving the disease, i.e., mitigating or ameliorating the disease and/or its symptoms or conditions. The term “treatment” as used herein can refer to both therapeutic treatment alone, prophylactic treatment alone, or both therapeutic and prophylactic treatment. Those in need of treatment (subjects in need thereof) can include those already with the disorder and/or those in which the disorder is to be prevented. As used herein, the term “treating”, can include inhibiting the disease, disorder or condition, e.g., impeding its progress; and relieving the disease, disorder, or condition, e.g., causing regression of the disease, disorder and/or condition. Treating the disease, disorder, or condition can include ameliorating at least one symptom of the particular disease, disorder, or condition, even if the underlying pathophysiology is not affected, e.g., such as treating the pain of a subject by administration of an analgesic agent even though such agent does not treat the cause of the pain.

As used herein, “dose,” “unit dose,” or “dosage” can refer to physically discrete units suitable for use in a subject, each unit containing a predetermined quantity of a disclosed compound and/or a pharmaceutical composition thereof calculated to produce the desired response or responses in association with its administration.

As used herein, “therapeutic” can refer to treating, healing, and/or ameliorating a disease, disorder, condition, or side effect, or to decreasing in the rate of advancement of a disease, disorder, condition, or side effect.

### Chemical Definitions

Compounds are described using standard nomenclature. Unless defined otherwise, all technical and scientific terms used herein have the same meaning as is commonly understood by one of skill in the art to which this invention belongs.

The compounds described herein include enantiomers, mixtures of enantiomers, diastereomers, tautomers, racemates and other isomers, such as rotamers, as if each is specifically described, unless otherwise indicated or otherwise excluded by context. It is to be understood that the compounds provided herein may contain chiral centers. Such chiral centers may be of either the (R-) or (S-) configuration. The compounds provided herein may either be enantiomerically pure, or be diastereomeric or enantiomeric mixtures. It is to be understood that the chiral centers of the compounds provided herein may undergo epimerization in vivo. As such, one of skill in the art will recognize that administration of a compound in its (R-) form is equivalent, for compounds that undergo epimerization in vivo, to administration of the compound in its (S-) form. Unless stated to the contrary, a formula with chemical bonds shown only as solid lines and not as wedges or dashed lines contemplates each possible isomer, e.g., each enantiomer, diastereomer, and meso compound, and a mixture of isomers, such as a racemic or scalemic mixture.

A dash (“-”) that is not between two letters or symbols is used to indicate a point of attachment for a substituent. For example, —(C═O)NH₂ is attached through the carbon of the keto (C═O) group.

The term “substituted”, as used herein, means that any one or more hydrogens on the designated atom or group is replaced with a moiety selected from the indicated group, provided that the designated atom's normal valence is not exceeded and the resulting compound is stable. For example, when the substituent is oxo (i.e., ═O) then two hydrogens on the atom are replaced. For example, a pyridyl group substituted by oxo is a pyridine. Combinations of substituents and/or variables are permissible only if such combinations result in stable compounds or useful synthetic intermediates. A stable active compound refers to a compound that can be isolated and can be formulated into a dosage form with a shelf life of at least one month. A stable manufacturing intermediate or precursor to an active compound is stable if it does not degrade within the period needed for reaction or other use. A stable moiety or substituent group is one that does not degrade, react or fall apart within the period necessary for use. Non-limiting examples of unstable moieties are those that combine heteroatoms in an unstable arrangement, as typically known and identifiable to those of skill in the art.

Any suitable group may be present on a “substituted” or “optionally substituted” position that forms a stable molecule and meets the desired purpose of the invention and includes, but is not limited to: alkyl, haloalkyl, alkoxy, alkenyl, alkynyl, aryl, heteroaryl, cycloalkyl, heterocycle, aldehyde, amino, carboxylic acid, ester, ether, halo, hydroxy, keto, nitro, cyano, azido, oxo, silyl, sulfo-oxo, sulfonyl, sulfone, sulfoxide, sulfonylamino, or thiol.

“Alkyl” is a straight chain or branched saturated aliphatic hydrocarbon group. In certain embodiments, the alkyl is C₁-C₂, C₁-C₃, or C₁-C₆ (i.e., the alkyl chain can be 1, 2, 3, 4, 5, or 6 carbons in length). The specified ranges as used herein indicate an alkyl group with length of each member of the range described as an independent species. For example, C₁-C₆alkyl as used herein indicates an alkyl group having from 1, 2, 3, 4, 5, or 6 carbon atoms and is intended to mean that each of these is described as an independent species and C₁-C₄alkyl as used herein indicates an alkyl group having from 1, 2, 3, or 4 carbon atoms and is intended to mean that each of these is described as an independent species. When C₀-Cₙalkyl is used herein in conjunction with another group, for example (C₃-C₇cycloalkyl)C₀-C₄alkyl, or —C₀-C₄(C₃-C₇cycloalkyl), the indicated group, in this case cycloalkyl, is either directly bound by a single covalent bond (C₀alkyl), or attached by an alkyl chain, in this case 1, 2, 3, or 4 carbon atoms. Alkyls can also be attached via other groups such as heteroatoms, as in —O—C₀-C₄alkyl(C₃-C₇cycloalkyl). Examples of alkyl include, but are not limited to, methyl, ethyl, n-propyl, isopropyl, n-butyl, isobutyl, sec-butyl, t-butyl, n-pentyl, isopentyl, tert-pentyl, neopentyl, n-hexyl, 2-methylpentane, 3-methylpentane, 2,2-dimethylbutane, and 2,3-dimethylbutane. In one embodiments, the alkyl group is optionally substituted as described herein.

“Alkenyl” is a straight or branched chain aliphatic hydrocarbon group having one or more carbon-carbon double bonds, each of which is independently either cis or trans, that may occur at a stable point along the chain. Non-limiting examples include C₂-C₄alkenyl and C₂-C₆alkenyl (i.e., having 2, 3, 4, 5, or 6 carbons). The specified ranges as used herein indicate an alkenyl group having each member of the range described as an independent species, as described above for the alkyl moiety. Examples of alkenyl include, but are not limited to, ethenyl and propenyl. In one embodiment, the alkenyl group is optionally substituted as described herein.

“Alkynyl” is a straight or branched chain aliphatic hydrocarbon group having one or more carbon-carbon triple bonds that may occur at any stable point along the chain, for example, C₂-C₄alkynyl or C₂-C₆alkynyl (i.e., having 2, 3, 4, 5, or 6 carbons). The specified ranges as used herein indicate an alkynyl group having each member of the range described as an independent species, as described above for the alkyl moiety. Examples of alkynyl include, but are not limited to, ethynyl, propynyl, 1-butynyl, 2-butynyl, 3-butynyl, 1-pentynyl, 2-pentynyl, 3-pentynyl, 4-pentynyl, 1-hexynyl, 2-hexynyl, 3-hexynyl, 4-hexynyl, and 5-hexynyl. In one embodiment, the alkynyl group is optionally substituted as described herein.

“Aryl” indicates an aromatic group containing only carbon in the aromatic ring or rings. In one embodiment, the aryl group contains 1 to 3 separate or fused rings and is 6 to 14 or 18 ring atoms, without heteroatoms as ring members. When indicated, such aryl groups may be further substituted with carbon or non-carbon atoms or groups. Such substitution may include fusion to a 4- to 7- or 5- to 7-membered saturated or partially unsaturated cyclic group that optionally contains 1, 2, or 3 heteroatoms independently selected from N, O, B, P, Si and S, to form, for example, a 3,4-methylenedioxyphenyl group. Aryl groups include, for example, phenyl and naphthyl, including 1-naphthyl and 2-naphthyl. In one embodiment, aryl groups are pendant. An example of a pendant ring is a phenyl group substituted with a phenyl group. In one embodiment, the aryl group is optionally substituted as described herein.

“Heteroaryl” refers to a stable monocyclic, bicyclic, or multicyclic aromatic ring which contains from 1 to 3, or in some embodiments 1, 2, or 3 heteroatoms selected from N, O, S, B, and P (and typically selected from N, O, and S) with remaining ring atoms being carbon, or a stable bicyclic or tricyclic system containing at least one 5, 6, or 7 membered aromatic ring which contains from 1 to 3, or in some embodiments from 1 to 2, heteroatoms selected from N, O, S, B, or P, with remaining ring atoms being carbon. In one embodiments, the only heteroatom is nitrogen. In one embodiment, the only heteroatom is oxygen. In one embodiment, the only heteroatom is sulfur. Monocyclic heteroaryl groups typically have from 5 to 6 ring atoms. In some embodiments, bicyclic heteroaryl groups are 8- to 10-membered heteroaryl groups, that is groups containing 8 or 10 ring atoms in which one 5-, 6-, or 7-membered aromatic ring is fused to a second aromatic or non-aromatic ring, wherein the point of attachment is the aromatic ring. When the total number of S and O atoms in the heteroaryl group excess 1, these heteroatoms are not adjacent to one another. In one embodiment, the total number of S and O atoms in the heteroaryl group is not more than 2. In another embodiment, the total number of S and O atoms in the heteroaryl group is not more than 1. Examples of heteroaryl groups include, but are not limited to, pyridinyl, imidazolyl, imidazopyridinyl, pyrimidinyl, pyrazolyl, triazolyl, pyrazinyl, furyl, thienyl, isoxazolyl, thiazolyl, oxadiazolyl, oxazolyl, isothiazolyl, pyrrolyl, quinolinyl, isoquinolinyl, tetrahydroisoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, triazolyl, thiadiazolyl, furazanyl, benzofurazanyl, benzothiophenyl, benzothiazolyl, benzoxazolyl, quinazolinyl, quinoxalinyl, naphthyridinyl, and furopyridinyl.

### Compounds

The present invention provides compounds that are useful in the treatment of infection with a virus, for example an infection with a coronavirus.

Thus, in one aspect, a compound is provided of Formula I-a

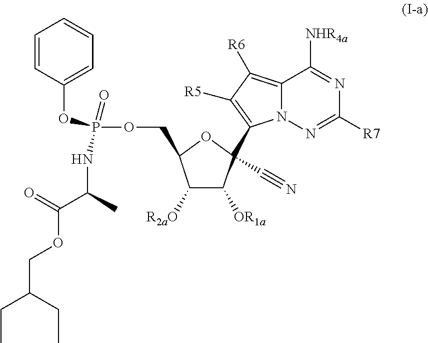

or a pharmaceutically acceptable salt thereof,
wherein:
R₁ₐ, R₂ₐ, and R₄ₐ are each independently selected from hydrogen, Zₐ—, and Zₐ-C(O)—, wherein at least one of R₁ₐ, R₂ₐ, and R₄ₐ is not hydrogen;
Zₐ is selected from Xₐ(CH₂)ₙYₐ(CH₂)ₙ—, Xₐ(CH₂)ₙYₐ(CH₂)ₙCH₂—, CH₃(CH₂)ₙCH(Br)—, CH₃(CH₂)ₙCH(Br)CH₂—, suramin, cellulose acetate, or an anionic polymer;
Xₐ is selected from CH₃, N₃, (C₁-C₆ alkyl)-S—, (aryl)-S—, (C₁-C₆ alkyl)-O—, (aryl)-O—, (C₁-C₆ alkyl)-NH—, (aryl)-NH—, Br, Cl, F, I, OH, NH₂, COOH, CHO, aryl, heteroaryl, C₂-C₆ alkynyl, C₂-C₆ alkenyl, suramin, cellulose acetate, or an anionic polymer;
Yₐ is selected from CH₂, O, S, NH, and heteroaryl;
n is independently selected at each occurrence from 0-18; and
R₅, R₆, and R₇ are independently selected from hydrogen, N₃, OH, CN, F, Cl, Br, I, NH₂, SH, C₁-C₆ alkyl, C₂-C₆ alkenyl, C₂-C₆ alkynyl, aryl, —O—(C₁-C₆ alkyl), and —O-(aryl).

In some embodiments of Formula I-a, R₁ₐ is Zₐ—.

In some embodiments of Formula I-a, R₁ₐ is Zₐ-C(O)—.

In some embodiments of Formula I-a, wherein R₂ₐ is Zₐ—.

In some embodiments of Formula I-a, wherein R₂ₐ is Zₐ—C(O)—.

In some embodiments of Formula I-a, wherein R₄ₐ is Zₐ—.

In some embodiments of Formula I-a, wherein R₄ₐ is Zₐ—C(O)—.

In some embodiments of Formula I-a, R₁ₐ is selected from Zₐ— and Zₐ-C(O)—, and R₂ₐ and R₄ₐ are each hydrogen.

In some embodiments of Formula I-a, R₂ₐ is selected from Zₐ— and Zₐ-C(O)—, and Ria and R₄ₐ are both hydrogen.

In some embodiments of Formula I-a, R₁ₐ and R₂ₐ are each independently selected from Zₐ— and Zₐ-C(O)—, and R₄ₐ is hydrogen.

In some embodiments of Formula I-a, R₄ₐ is selected from Zₐ— and Zₐ-C(O)—, and R₁ₐ and R₂ₐ are both hydrogen.

In some embodiments of Formula I-a, Zₐ is selected from Xₐ(CH₂)ₙYₐ(CH₂)ₙ— and Xₐ(CH₂)ₙYₐ(CH₂)ₙCH₂—.

In some embodiments of Formula I-a, Xₐ is CH₃.

In some embodiments of Formula I-a, wherein Xₐ is N₃.

In some embodiments of Formula I-a, Xₐ is CH₃CH₂S.

In some embodiments of Formula I-a, Yₐ is CH₂.

In some embodiments of Formula I-a, Yₐ is O.

In some embodiments of Formula I-a, Zₐ is selected from CH₃(CH₂)₁₁—, CH₃(CH₂)₁₂—, CH₃(CH₂)₁₃—, CH₃(CH₂)₁₄—, CH₃(CH₂)₁₆—, N₃(CH₂)₁₁—, CH₃(CH₂)₉O(CH₂)₂—, CH₃(CH₂)₁₁O(CH₂)₂—, and CH₃CH₂S(CH₂)₁₁—.

In some embodiments of Formula I-a, R₅ is hydrogen.

In some embodiments of Formula I-a, R₆ is hydrogen.

In some embodiments of Formula I-a, R₇ is hydrogen.

In some embodiments, the compound is selected from:

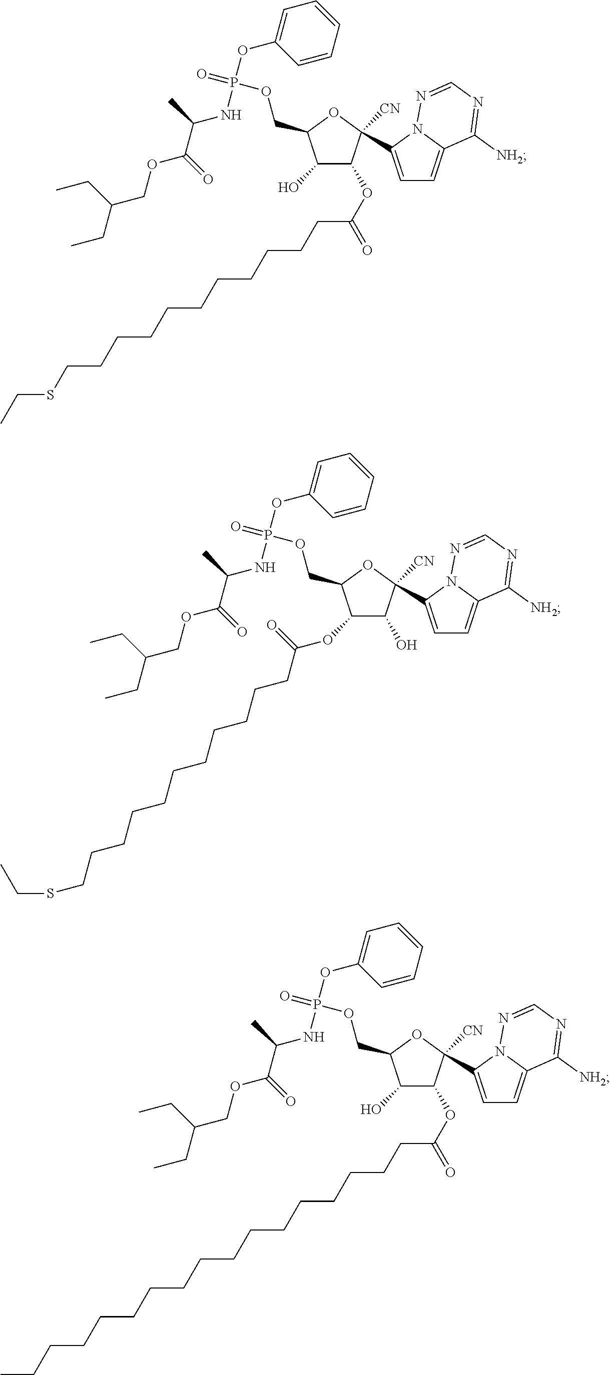 [SMILES: CCSCCCCCCCCCCCC(=O)O[C@@H]1[C@H](O)[C@@H](COP(=O)(N[C@H](C)C(=O)OCC(CC)CC)Oc2ccccc2)O[C@@]1(C#N)c1ccc2c(N)ncnn12 ; CCSCCCCCCCCCCCC(=O)O[C@H]1[C@@H](O)[C@](C#N)(c2ccc3c(N)ncnn23)O[C@@H]1COP(=O)(N[C@H](C)C(=O)OCC(CC)CC)Oc1ccccc1 ; CCCCCCCCCCCCCCCCCC(=O)O[C@@H]1[C@H](O)[C@@H](COP(=O)(N[C@H](C)C(=O)OCC(CC)CC)Oc2ccccc2)O[C@@]1(C#N)c1ccc2c(N)ncnn12]

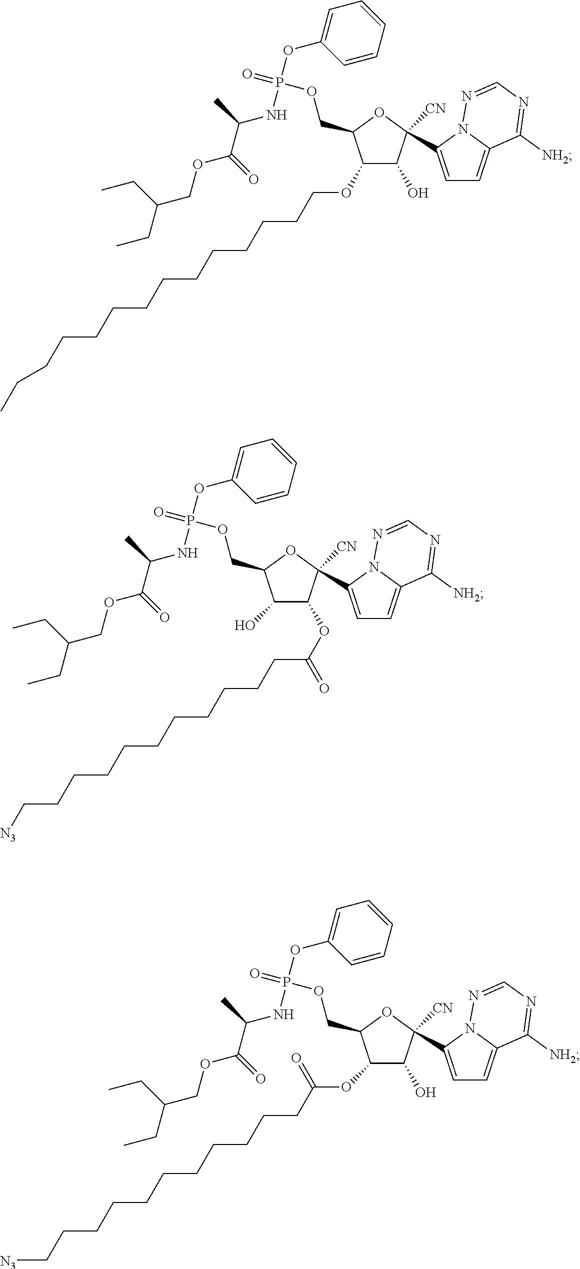 [SMILES: CCCCCCCCCCCCCCO[C@H]1[C@@H](O)[C@](C#N)(c2ccc3c(N)ncnn23)O[C@@H]1COP(=O)(N[C@H](C)C(=O)OCC(CC)CC)Oc1ccccc1 ; CCC(CC)COC(=O)[C@@H](C)NP(=O)(OC[C@H]1O[C@@](C#N)(c2ccc3c(N)ncnn23)[C@H](OC(=O)CCCCCCCCCCCN=[N+]=[N-])[C@@H]1O)Oc1ccccc1 ; CCC(CC)COC(=O)[C@@H](C)NP(=O)(OC[C@H]1O[C@@](C#N)(c2ccc3c(N)ncnn23)[C@H](O)[C@@H]1OC(=O)CCCCCCCCCCCN=[N+]=[N-])Oc1ccccc1]

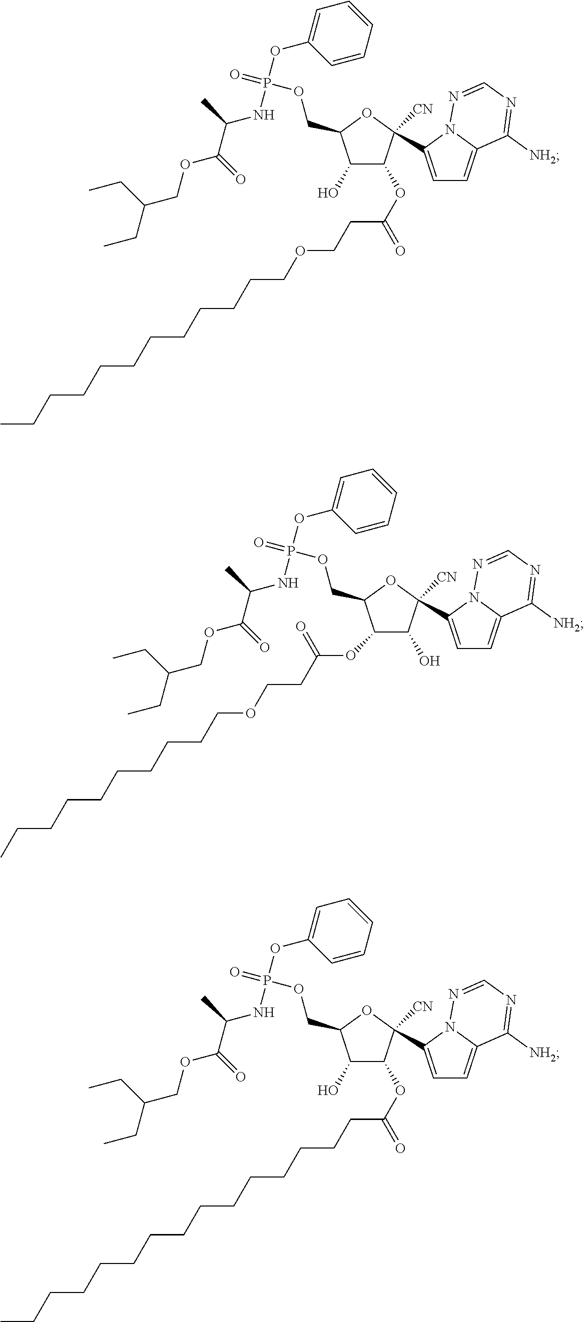 [SMILES: CCCCCCCCCCCCOCCC(=O)O[C@@H]1[C@H](O)[C@@H](COP(=O)(N[C@H](C)C(=O)OCC(CC)CC)Oc2ccccc2)O[C@@]1(C#N)c1ccc2c(N)ncnn12 ; CCCCCCCCCCOCCC(=O)O[C@H]1[C@@H](O)[C@](C#N)(c2ccc3c(N)ncnn23)O[C@@H]1COP(=O)(N[C@H](C)C(=O)OCC(CC)CC)Oc1ccccc1 ; CCCCCCCCCCCCCCCC(=O)O[C@@H]1[C@H](O)[C@@H](COP(=O)(N[C@H](C)C(=O)OCC(CC)CC)Oc2ccccc2)O[C@@]1(C#N)c1ccc2c(N)ncnn12]

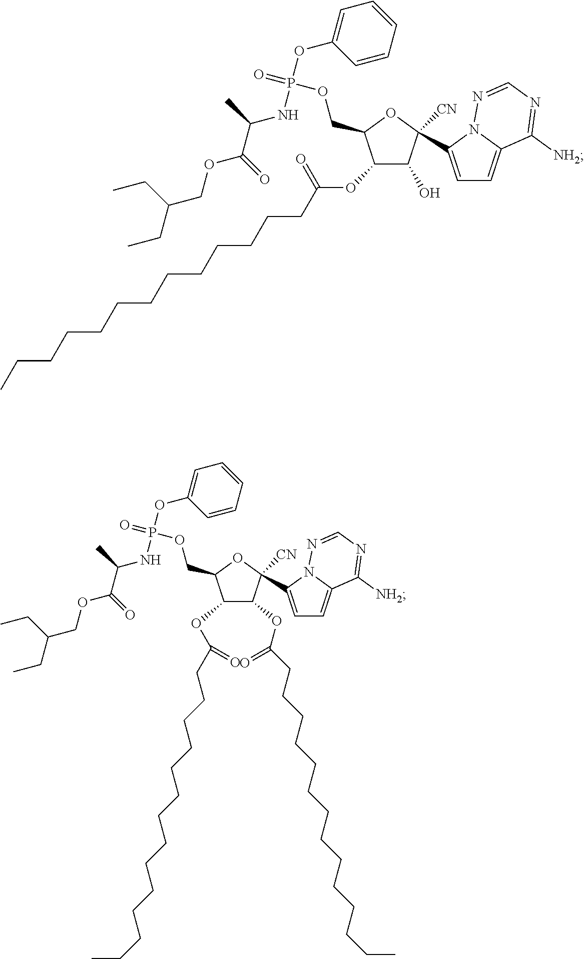 [SMILES: CCCCCCCCCCCCCC(=O)O[C@H]1[C@@H](O)[C@](C#N)(c2ccc3c(N)ncnn23)O[C@@H]1COP(=O)(N[C@H](C)C(=O)OCC(CC)CC)Oc1ccccc1 ; CCCCCCCCCCCCCCC(=O)O[C@H]1[C@@H](OC(=O)CCCCCCCCCCCCCC)[C@](C#N)(c2ccc3c(N)ncnn23)O[C@@H]1COP(=O)(N[C@H](C)C(=O)OCC(CC)CC)Oc1ccccc1]

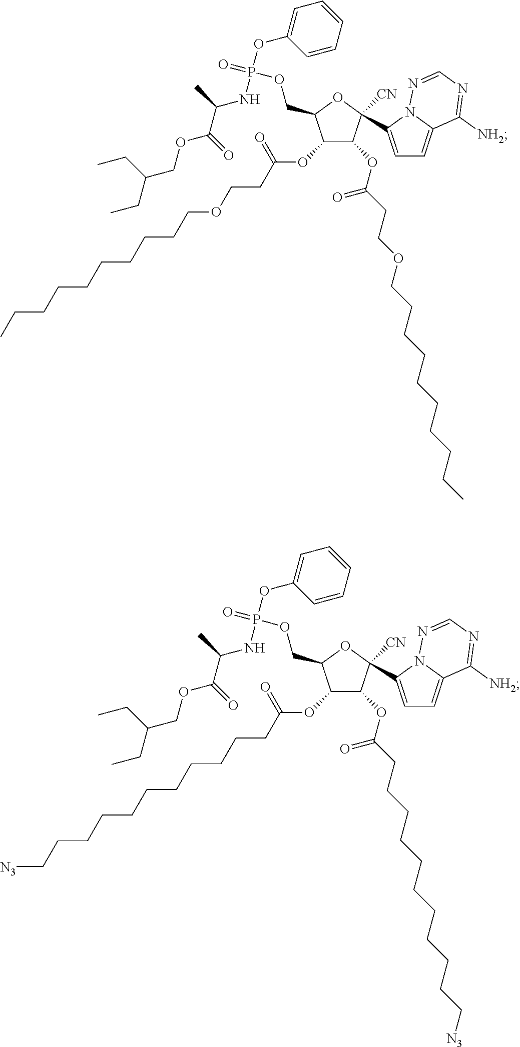 [SMILES: CCCCCCCCCCOCCC(=O)O[C@H]1[C@@H](OC(=O)CCOCCCCCCCCCC)[C@](C#N)(c2ccc3c(N)ncnn23)O[C@@H]1COP(=O)(N[C@H](C)C(=O)OCC(CC)CC)Oc1ccccc1 ; CCC(CC)COC(=O)[C@@H](C)NP(=O)(OC[C@H]1O[C@@](C#N)(c2ccc3c(N)ncnn23)[C@H](OC(=O)CCCCCCCCCCCN=[N+]=[N-])[C@@H]1OC(=O)CCCCCCCCCCCN=[N+]=[N-])Oc1ccccc1]

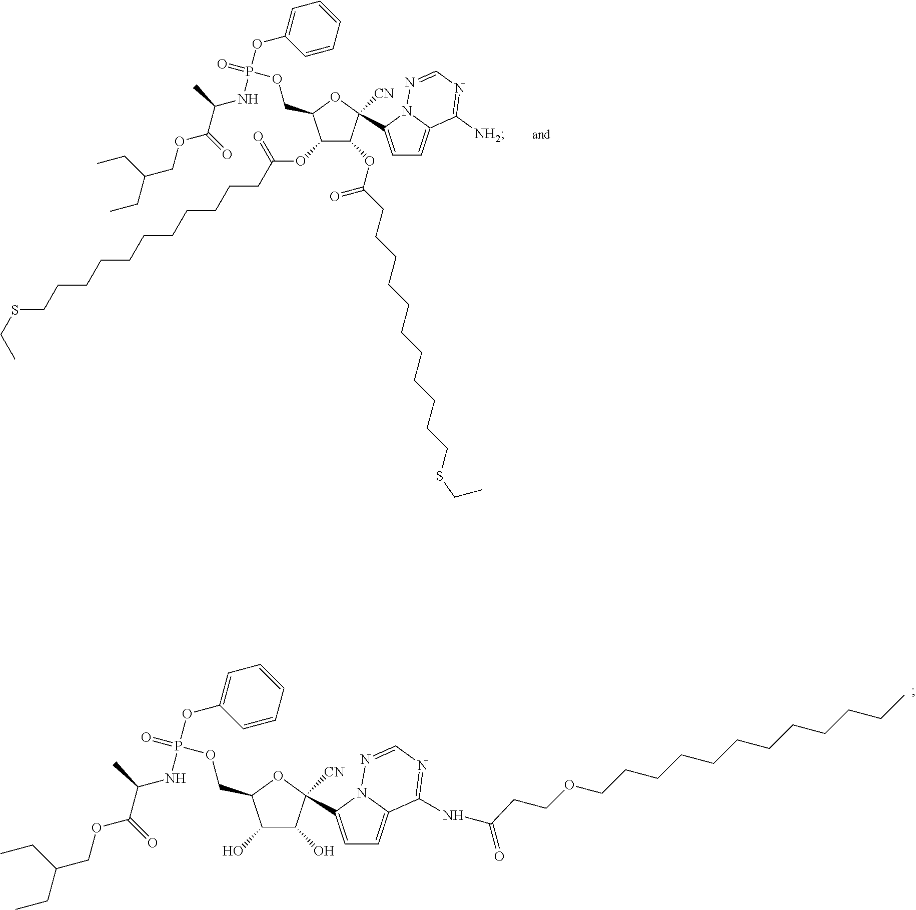 [SMILES: CCSCCCCCCCCCCCC(=O)O[C@H]1[C@@H](OC(=O)CCCCCCCCCCCSCC)[C@](C#N)(c2ccc3c(N)ncnn23)O[C@@H]1COP(=O)(N[C@H](C)C(=O)OCC(CC)CC)Oc1ccccc1 ; CCCCCCCCCCCCOCCC(=O)Nc1ncnn2c([C@]3(C#N)O[C@H](COP(=O)(N[C@H](C)C(=O)OCC(CC)CC)Oc4ccccc4)[C@@H](O)[C@H]3O)ccc12]

or a pharmaceutically acceptable salt thereof.

In another aspect, a compound is provided of Formula II-a

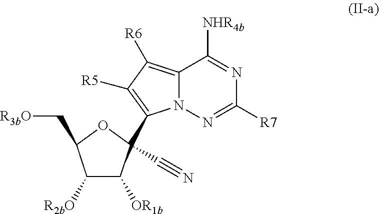

or a pharmaceutically acceptable salt thereof;
wherein:
R_{1b}, R_{2b}, R_{3b}, and R_{4b} are each independently selected from hydrogen, Zₐ—, and Zₐ-C(O)—, wherein at least one of R_{1b}, R_{2b}, R_{3b}, and R_{4b} is not hydrogen;
Zₐ is selected from Xₐ(CH₂)ₙYₐ(CH₂)ₙ—, Xₐ(CH₂)ₙYₐ(CH₂)ₙCH₂—, CH₃(CH₂)ₙCH(Br)—, CH₃(CH₂)ₙCH(Br)CH₂—, suramin, cellulose acetate, or an anionic polymer;
Xₐ is selected from CH₃, N₃, (C₁-C₆ alkyl)-S—, (aryl)-S—, (C₁-C₆ alkyl)-O—, (aryl)-O—, (C₁-C₆ alkyl)-NH—, (aryl)-NH—, Br, Cl, F, I, OH, NH₂, COOH, CHO, aryl, heteroaryl, C₂-C₆ alkynyl, C₂-C₆ alkenyl, suramin, cellulose acetate, or an anionic polymer;
Yₐ is selected from CH₂, O, S, NH, and heteroaryl;
n is independently selected at each occurrence from 0-18; and
R₅, R₆, and R₇ are independently selected from hydrogen, N₃, OH, CN, F, Cl, Br, I, NH₂, SH, C₁-C₆ alkyl, C₂-C₆ alkenyl, C₂-C₆ alkynyl, aryl, —O—(C₁-C₆ alkyl), and —O-(aryl).

In some embodiments of Formula II-a, R_{1b} is Zₐ—.

In some embodiments of Formula II-a, R_{1b} is Zₐ—C(O)—.

In some embodiments of Formula II-a, R_{2b} is Zₐ—.

In some embodiments of Formula II-a, R_{2b} is Zₐ—C(O)—.

In some embodiments of Formula II-a, R_{3b} is Zₐ—.

In some embodiments of Formula II-a, R_{3b} is Zₐ—C(O)—.

In some embodiments of Formula II-a, R_{4b} is Zₐ—.

In some embodiments of Formula II-a, R_{4b} is Zₐ—C(O)—.

In some embodiments of Formula II-a, R_{1b} is selected from Zₐ— and Zₐ—C(O)—, and R_{2b}, R_{3b}, and R_{4b} are all hydrogen.

In some embodiments of Formula II-a, R_{2b} is selected from Zₐ— and Zₐ—C(O)—, and R_{1b}, R_{3b}, and R_{4b} are all hydrogen.

In some embodiments of Formula II-a, R_{3b} is selected from Zₐ— and Zₐ—C(O)—, and R_{1b}, R_{2b}, and R_{4b} are all hydrogen.

In some embodiments of Formula II-a, R_{4b} is selected from Zₐ— and Zₐ—C(O)—, and R_{1b}, R_{2b}, and R_{3b} are all hydrogen.

In some embodiments of Formula II-a, Zₐ is selected from Xₐ(CH₂)ₙYₐ(CH₂)ₙ— and Xₐ(CH₂)ₙYₐ(CH₂)ₙCH₂—.

In some embodiments of Formula II-a, Xₐ is CH₃.

In some embodiments of Formula II-a, Xₐ is N₃.

In some embodiments of Formula II-a, Xₐ is CH₃CH₂S.

In some embodiments of Formula II-a, Yₐ is CH₂.

In some embodiments of Formula II-a, Yₐ is O.

In some embodiments of Formula II-a, Zₐ is selected from CH₃(CH₂)₁₁—, CH₃(CH₂)₁₂—, CH₃(CH₂)₁₃—, CH₃(CH₂)₁₄—, CH₃(CH₂)₁₆—, N₃(CH₂)₁₁—, CH₃(CH₂)₉O(CH₂)₂—, CH₃(CH₂)₁₁O(CH₂)₂—, and CH₃CH₂S(CH₂)₁₁—.

In some embodiments of Formula II-a, R₅ is hydrogen.

In some embodiments of Formula II-a, R₆ is hydrogen.

In some embodiments of Formula II-a, R₇ is hydrogen.

In another aspect, a compound is provided comprising at least one substituted nucleoside selected from the group consisting of Formulas I or II

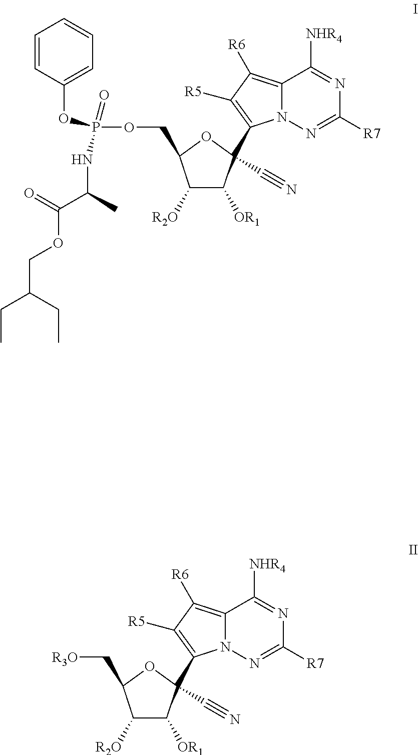

wherein

R₁, R₂, and R₃ are each independently selected from Z-CO—, an anionic polymer complexed with a cleavable linker, a fatty acid analog complexed with a cleavable linker, a fatty alcohol analog complexed with a cleavable linker, a carboxylic ester side chain of a linear or cyclic peptide, a polycarboxylic ester aryl or heteroaryl, carbopol, or a phosphodiester; wherein

the fatty acid analog is selected from the group consisting of X′(CH₂)ₙY′(CH₂)nCO- and CH₃(CH₂)ₙCH(Br)CO—;

the fatty alcohol analog is selected from the group consisting of X′_{(CH2)}nY′(CH₂)ₙCH₂O- and CH₃(CH₂)ₙCH(Br)CH₂O—;

Z is selected from X(CH₂)ₙY(CH₂)ₙ—, CH₃(CH₂)ₙCH(Br)—, suramin, cellulose acetate, or an anionic polymer;

X is selected from CH₃, N₃, alkyl-S, alkyl-O, aryl-O, aryl-S, alkyl-NH, aryl-NH, Br, Cl, F, I, OH, NH₂, COOH, CHO, CH₃S, aryl, heteroaryl, phenyl, substituted phenyl, alkyne, alkene, suramin, cellulose acetate, or an anionic polymer;

X′ is selected from CH₃, N₃, alkyl-S, alkyl-O, aryl-O, aryl-S, alkyl-NH, aryl-NH, Br, Cl, F, I, OH, NH₂, COOH, CHO, CH₃S, aryl, heteroaryl, phenyl, substituted phenyl, suramin, cellulose acetate, or an anionic polymer;

Y is selected from CH₂, O, S, NH, 1,2,3-triazole;

Y′ is selected from CH₂, O, S, NH;

independently, n is selected from 0-18;

R₄ is selected from H or R₁; and

R₅, R₆, and R₇ are each independently selected from H, N₃, F, CN, Cl, Br, F, I, OH, NH₂, SH alkyl, alkene, alkyne, aryl, O-alkyl, O-aryl.

A “pharmaceutically acceptable salt” is a derivative of the disclosed compound in which the parent compound is modified by making inorganic and organic, pharmaceutically acceptable, acid or base addition salts thereof. The salts of the present compounds can be synthesized from a parent compound that contains a basic or acidic moiety by conventional chemical methods. Generally, such salts can be prepared by reacting free acid forms of these compounds with a stoichiometric amount of the appropriate base (such as Na, Ca, Mg, or K hydroxide, carbonate, bicarbonate, or the like), or by reacting free base forms of these compounds with a stoichiometric amount of the appropriate acid. Such reactions are typically carried out in water or in an organic solvent, or in a mixture of the two. Generally, non-aqueous media like ether, ethyl acetate, ethanol, isopropanol, or acetonitrile are typical, where practicable. Salts of the present compounds further include solvates of the compounds and of the compound salts. Examples of pharmaceutically acceptable salts include, but are not limited to, mineral or organic acid salts of basic residues such as amines; alkali or organic salts of acidic residues such as carboxylic acids; and the like. The pharmaceutically acceptable salts include salts which are acceptable for human consumption and the quaternary ammonium salts of the parent compound formed, for example, from inorganic or organic salts. Example of such salts include, but are not limited to, those derived from inorganic acids such as hydrochloric, hydrobromic, sulfuric, sulfamic, phosphoric, nitric, and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicyclic, mesylic, esylic, besylic, sulfanilic, 2-acetoxybenzoic, fumaric, toluenesulfonic, methanesulfone, ethane disulfonic, oxalic, isethionic, HOOC—(CH₂)₁₋₄—COOH, and the like, or using a different acid that produced the same counterion. Lists of additional suitable salts may be found, e.g., in Remington's Pharmaceutical Sciences, 17^{th} ed., Mack Publishing Company, Easton, Pa., p. 1418 (1985).

As used herein, substantially pure means sufficiently homogeneous to appear free of readily detectable impurities as determined by standard methods of analysis, such as thin layer chromatography (TLC), nuclear magnetic resonance (NMR), gel electrophoresis, high performance liquid chromatography (HPLC) and mass spectrometry (MS), gas-chromatography mass spectrometry (GC-MS), and similar, used by those of skill in the art to assess such purity, or sufficiently pure such that further purification would not detectably alter the physical and chemical properties, such as enzymatic and biological activities, of the substance. Both traditional and modern methods for purification of the compounds to produce substantially chemically pure compounds are known to those of skill in the art. A substantially chemically pure compound may, however, be a mixture of stereoisomers.

The present disclosure also includes compounds described herein with at least one desired isotopic substitution of an atom, at an amount above the natural abundance of the isotope, i.e., enriched.

Examples of isotopes that can be incorporated into compounds of the present disclosure include isotopes of hydrogen, carbon, nitrogen, oxygen, phosphorous, fluorine, and chlorine, such as ²H, ³H, ¹¹C, ¹³C, ¹⁵N, ¹⁷O, ¹⁸O, ¹⁸F, ³¹P, ³²P, ³⁵S, ³⁶Cl, and ¹²⁵I, respectively. In one embodiment, isotopically labeled compounds can be used in metabolic studies (with ¹⁴C), reaction kinetic studies (with, for example ²H or ³H), detection or imaging techniques, such as positron emission tomography (PET) or single-photon emission computed tomography (SPECT) including drug and substrate tissue distribution assays, or in radioactive treatment of patients. In particular, an ¹⁸F labeled compound may be particularly desirable for PET or SPECT studies. Isotopically labeled compounds of this invention and prodrugs thereof can generally be prepared by carrying out the procedures disclosed herein by substituting a readily available isotopically labeled reagent for a non-isotopically labeled reagent.

By way of general example and without limitation, isotopes of hydrogen, for example deuterium (²H) and tritium (³H) may optionally be used anywhere in described structures that achieves the desired result. Alternatively or in addition, isotopes of carbon, e.g., ¹³C and ¹⁴C, may be used. In one embodiment, the isotopic substitution is replacing hydrogen with a deuterium at one or more locations on the molecule to improve the performance of the molecule as a drug, for example, the pharmacodynamics, pharmacokinetics, biodistribution, half-life, stability, AUC, Tₘₐₓ, Cₘₐₓ, etc. For example, the deuterium can be bound to carbon in allocation of bond breakage during metabolism (an alpha-deuterium kinetic isotope effect) or next to or near the site of bond breakage (a beta-deuterium kinetic isotope effect).

Isotopic substitutions, for example deuterium substitutions, can be partial or complete. Partial deuterium substitution means that at least one hydrogen is substituted with deuterium. In certain embodiments, the isotope is 80, 85, 90, 95, or 99% or more enriched in an isotope at any location of interest. In some embodiments, deuterium is 80, 85, 90, 95, or 99% enriched at a desired location. Unless otherwise stated, the enrichment at any point is above natural abundance, and in an embodiment is enough to alter a detectable property of the compounds as a drug in a human.

The compounds of the present disclosure may form a solvate with solvents (including water). Therefore, in one embodiment, the invention includes a solvated form of the active compound. The term “solvate” refers to a molecular complex of a compound of the present invention (including a salt thereof) with one or more solvent molecules. Non-limiting examples of solvents are water, ethanol, dimethyl sulfoxide, acetone and other common organic solvents. The term “hydrate” refers to a molecular complex comprising a disclosed compound and water. Pharmaceutically acceptable solvates in accordance with the invention include those wherein the solvent of crystallization may be isotopically substituted, e.g., D₂O, d6-acetone, or d6-DMSO. A solvate can be in a liquid or solid form.

A “prodrug” as used herein means a compound which when administered to a host in vivo is converted into a parent drug. As used herein, the term “parent drug” means any of the presently described compounds herein. Prodrugs can be used to achieve any desired effect, including to enhance properties of the parent drug or to improve the pharmaceutic or pharmacokinetic properties of the parent, including to increase the half-life of the drug in vivo. Prodrug strategies provide choices in modulating the conditions for in vivo generation of the parent drug. Non-limiting examples of prodrug strategies include covalent attachment of removable groups, or removable portions of groups, for example, but not limited to, acylating, phosphorylation, phosphonylation, phosphoramidate derivatives, amidation, reduction, oxidation, esterification, alkylation, other carboxy derivatives, sulfoxy or sulfone derivatives, carbonylation, or anhydrides, among others. In certain embodiments, the prodrug renders the parent compound more lipophilic. In certain embodiments, a prodrug can be provided that has several prodrug moieties in a linear, branched, or cyclic manner. For example, non-limiting embodiments include the use of a divalent linker moiety such as a dicarboxylic acid, amino acid, diamine, hydroxycarboxylic acid, hydroxyamine, dihydroxy compound, or other compound that has at least two functional groups that can link the parent compound with another prodrug moiety, and is typically biodegradable in vivo. In some embodiments, 2, 3, 4, or 5 prodrug biodegradable moieties are covalently bound in a sequence, branched, or cyclic fashion to the parent compound. Non-limiting examples of prodrugs according to the present disclosure are formed with: a carboxylic acid on the parent drug and a hydroxylated prodrug moiety to form an ester; a carboxylic acid on the parent drug and an amine prodrug to form an amide; an amino on the parent drug and a carboxylic acid prodrug moiety to form an amide; an amino on the parent drug and a sulfonic acid to form a sulfonamide; a sulfonic acid on the parent drug and an amino on the prodrug moiety to form a sulfonamide; a hydroxyl group on the parent drug and a carboxylic acid on the prodrug moiety to form an ester; a hydroxyl on the parent drug and a hydroxylated prodrug moiety to form an ester; a phosphonate on the parent drug and a hydroxylated prodrug moiety to form a phosphonate ester; a phosphoric acid on the parent drug and a hydroxylated prodrug moiety to form a phosphate ester; a hydroxyl on the parent drug and a phosphonate on the prodrug to form a phosphonate ester; a hydroxyl on the parent drug and a phosphoric acid prodrug moiety to form a phosphate ester; a carboxylic acid on the parent drug and a prodrug of the structure HO—(CH₂)₂—O—(C₂₋₂₄ alkyl) to form an ester; a carboxylic acid on the parent drug and a prodrug of the structure HO—(CH₂)₂—S—(C₂₋₂₄ alkyl) to form a thioester; a hydroxyl on the parent drug and a prodrug of the structure HO—(CH₂)₂—O—(C₂₋₂₄ alkyl) to form an ether; a hydroxyl on the parent drug and a prodrug of the structure HO—(CH₂)₂—O—(C₂₋₂₄ alkyl) to form an thioether; and a carboxylic acid, oxime, hydrazide, hydrazine, amine or hydroxyl on the parent compound and a prodrug moiety that is a biodegradable polymer or oligomer including but not limited to polylactic acid, polylactide-co-glycolide, polyglycolide, polyethylene glycol, polyanhydride, polyester, polyamide, or a peptide.

In some embodiments, a prodrug is provided by attaching a natural or non-natural amino acid to an appropriate functional moiety on the parent compound, for example, oxygen, nitrogen, or sulfur, and typically oxygen or nitrogen, usually in a manner such that the amino acid is cleaved in vivo to provide the parent drug. The amino acid can be used alone or covalently linked (straight, branched or cyclic) to one or more other prodrug moieties to modify the parent drug to achieve the desired performance, such as increased half-life, lipophilicity, or other drug delivery or pharmacokinetic properties. The amino acid can be any compound with an amino group and a carboxylic acid, which includes an aliphatic amino acid, alkyl amino acid, aromatic amino acid, heteroaliphatic amino acid, heteroalkyl amino acid, heterocyclic amino acid, or heteroaryl amino acid.

### Pharmaceutical Compositions

The compounds as used in the methods described herein can be administered by any suitable method and technique presently or prospectively known to those skilled in the art. For example, the active components described herein can be formulated in a physiologically- or pharmaceutically-acceptable form and administered by any suitable route known in the art including, for example, oral and parenteral routes of administering. As used herein, the term “parenteral” includes subcutaneous, intradermal, intravenous, intramuscular, intraperitoneal, and intrasternal administration, such as by injection. Administration of the active components of their compositions can be a single administration, or at continuous and distinct intervals as can be readily determined by a person skilled in the art.

Compositions, as described herein, comprising an active compound and a pharmaceutically acceptable carrier or excipient of some sort may be useful in a variety of medical and non-medical applications. For example, pharmaceutical compositions comprising an active compound and an excipient may be useful for the treatment or prevention of an infection with a virus in a subject in need thereof.

“Pharmaceutically acceptable carrier” (sometimes referred to as a “carrier”) means a carrier or excipient that is useful in preparing a pharmaceutical or therapeutic composition that is generally safe and non-toxic and includes a carrier that is acceptable for veterinary and/or human pharmaceutical or therapeutic use. The terms “carrier” or “pharmaceutically acceptable carrier” can include, but are not limited to, phosphate buffered saline solution, water, emulsions (such as an oil/water or water/oil emulsion) and/or various types of wetting agents. As used herein, the term “carrier” encompasses, but is not limited to, any excipient, diluent, filler, salt, buffer, stabilizer, solubilizer, lipid, stabilizer, or other material well known in the art for use in pharmaceutical formulations and as described further herein.

“Excipients” include any and all solvents, diluents or other liquid vehicles, dispersion or suspension aids, surface active agents, isotonic agents, thickening or emulsifying agents, preservatives, solid binders, lubricants and the like, as suited to the particular dosage form desired. General considerations in formulation and/or manufacture can be found, for example, in Remington's Pharmaceutical Sciences, Sixteenth Edition, E. W. Martin (Mack Publishing Co., Easton, Pa., 1980), and Remington: The Science and Practice of Pharmacy, 21st Edition (Lippincott Williams & Wilkins, 2005).

Exemplary excipients include, but are not limited to, any non-toxic, inert solid, semisolid or liquid filler, diluent, encapsulating material or formulation auxiliary of any type. Some examples of materials which can serve as excipients include, but are not limited to, sugars such as lactose, glucose, and sucrose; starches such as corn starch and potato starch; cellulose and its derivatives such as sodium carboxymethyl cellulose, ethyl cellulose, and cellulose acetate; powdered tragacanth; malt; gelatin; talc; excipients such as cocoa butter and suppository waxes; oils such as peanut oil, cottonseed oil; safflower oil; sesame oil; olive oil; corn oil and soybean oil; glycols such as propylene glycol; esters such as ethyl oleate and ethyl laurate; agar; detergents such as Tween 80; buffering agents such as magnesium hydroxide and aluminum hydroxide; alginic acid; pyrogen-free water; isotonic saline; Ringer's solution; ethyl alcohol; and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator. As would be appreciated by one of skill in this art, the excipients may be chosen based on what the composition is useful for. For example, with a pharmaceutical composition or cosmetic composition, the choice of the excipient will depend on the route of administration, the agent being delivered, time course of delivery of the agent, etc., and can be administered to humans and/or to animals, orally, rectally, parenterally, intracisternally, intravaginally, intranasally, intraperitoneally, topically (as by powders, creams, ointments, or drops), buccally, or as an oral or nasal spray. In some embodiments, the active compounds disclosed herein are administered topically.

Exemplary diluents include calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, etc., and combinations thereof.

Exemplary granulating and/or dispersing agents include potato starch, corn starch, tapioca starch, sodium starch glycolate, clays, alginic acid, guar gum, citrus pulp, agar, bentonite, cellulose and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (Veegum), sodium lauryl sulfate, quaternary ammonium compounds, etc., and combinations thereof.

Exemplary surface active agents and/or emulsifiers include natural emulsifiers (e.g. acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g. bentonite [aluminum silicate] and Veegum [magnesium aluminum silicate]), long chain amino acid derivatives, high molecular weight alcohols (e.g. stearyl alcohol, cetyl alcohol, oleyl alcohol, triacetin monostearate, ethylene glycol distearate, glyceryl monostearate, and propylene glycol monostearate, polyvinyl alcohol), carbomers (e.g. carboxy polymethylene, polyacrylic acid, acrylic acid polymer, and carboxy vinyl polymer), carrageenan, cellulosic derivatives (e.g. carboxymethylcellulose sodium, powdered cellulose, hydroxymethyl cellulose, hydroxypropyl cellulose, hydroxypropyl methylcellulose, methylcellulose), sorbitan fatty acid esters (e.g. polyoxyethylene sorbitan monolaurate [Tween 20], polyoxyethylene sorbitan [Tween 60], polyoxyethylene sorbitan monooleate [Tween 80], sorbitan monopalmitate [Span 40], sorbitan monostearate [Span 60], sorbitan tristearate [Span 65], glyceryl monooleate, sorbitan monooleate [Span 80]), polyoxyethylene esters (e.g. polyoxyethylene monostearate [Myrj 45], polyoxyethylene hydrogenated castor oil, polyethoxylated castor oil, polyoxymethylene stearate, and Solutol), sucrose fatty acid esters, polyethylene glycol fatty acid esters (e.g. Cremophor), polyoxyethylene ethers, (e.g. polyoxyethylene lauryl ether [Brij 30]), poly(vinyl-pyrrolidone), diethylene glycol monolaurate, triethanolamine oleate, sodium oleate, potassium oleate, ethyl oleate, oleic acid, ethyl laurate, sodium lauryl sulfate, Pluronic F 68, Poloxamer 188, cetrimonium bromide, cetylpyridinium chloride, benzalkonium chloride, docusate sodium, etc. and/or combinations thereof. Exemplary binding agents include starch (e.g. cornstarch and starch paste), gelatin, sugars (e.g. sucrose, glucose, dextrose, dextrin, molasses, lactose, lactitol, mannitol, etc.), natural and synthetic gums (e.g. acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (Veegum), and larch arabogalactan), alginates, polyethylene oxide, polyethylene glycol, inorganic calcium salts, silicic acid, polymethacrylates, waxes, water, alcohol, etc., and/or combinations thereof.

Exemplary preservatives include antioxidants, chelating agents, antimicrobial preservatives, antifungal preservatives, alcohol preservatives, acidic preservatives, and other preservatives.

Exemplary antioxidants include alpha tocopherol, ascorbic acid, ascorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and sodium sulfite.

Exemplary chelating agents include ethylenediaminetetraacetic acid (EDTA) and salts and hydrates thereof (e.g., sodium edetate, disodium edetate, trisodium edetate, calcium disodium edetate, dipotassium edetate, and the like), citric acid and salts and hydrates thereof (e.g., citric acid monohydrate), fumaric acid and salts and hydrates thereof, malic acid and salts and hydrates thereof, phosphoric acid and salts and hydrates thereof, and tartaric acid and salts and hydrates thereof. Exemplary antimicrobial preservatives include benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and thimerosal.

Exemplary antifungal preservatives include butyl paraben, methyl paraben, ethyl paraben, propyl paraben, benzoic acid, hydroxybenzoic acid, potassium benzoate, potassium sorbate, sodium benzoate, sodium propionate, and sorbic acid.

Exemplary alcohol preservatives include ethanol, polyethylene glycol, phenol, phenolic compounds, bisphenol, chlorobutanol, hydroxybenzoate, and phenylethyl alcohol.

Exemplary acidic preservatives include vitamin A, vitamin C, vitamin E, beta-carotene, citric acid, acetic acid, dehydroacetic acid, ascorbic acid, sorbic acid, and phytic acid. Other preservatives include tocopherol, tocopherol acetate, deteroxime mesylate, cetrimide, butylated hydroxyanisol (BHA), butylated hydroxytoluene (BHT), ethylenediamine, sodium lauryl sulfate (SLS), sodium lauryl ether sulfate (SLES), sodium bisulfite, sodium metabisulfite, potassium sulfite, potassium metabisulfite, Glydant Plus, Phenonip, methylparaben, Germall 115, Germaben II, Neolone, Kathon, and Euxyl. In certain embodiments, the preservative is an anti-oxidant. In other embodiments, the preservative is a chelating agent.

Exemplary buffering agents include citrate buffer solutions, acetate buffer solutions, phosphate buffer solutions, ammonium chloride, calcium carbonate, calcium chloride, calcium citrate, calcium glubionate, calcium gluceptate, calcium gluconate, D-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen-free water, isotonic saline, Ringer's solution, ethyl alcohol, etc., and combinations thereof.

Exemplary lubricating agents include magnesium stearate, calcium stearate, stearic acid, silica, talc, malt, glyceryl behanate, hydrogenated vegetable oils, polyethylene glycol, sodium benzoate, sodium acetate, sodium chloride, leucine, magnesium lauryl sulfate, sodium lauryl sulfate, etc., and combinations thereof.

Exemplary natural oils include almond, apricot kernel, avocado, babassu, bergamot, black current seed, borage, cade, chamomile, canola, caraway, carnauba, castor, cinnamon, cocoa butter, coconut, cod liver, coffee, corn, cotton seed, emu, eucalyptus, evening primrose, fish, flaxseed, geraniol, gourd, grape seed, hazel nut, hyssop, isopropyl myristate, jojoba, kukui nut, lavandin, lavender, lemon, litsea cubeba, macademia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils. Exemplary synthetic oils include, but are not limited to, butyl stearate, caprylic triglyceride, capric triglyceride, cyclomethicone, diethyl sebacate, dimethicone 360, isopropyl myristate, mineral oil, octyldodecanol, oleyl alcohol, silicone oil, and combinations thereof.

Additionally, the composition may further comprise a polymer. Exemplary polymers contemplated herein include, but are not limited to, cellulosic polymers and copolymers, for example, cellulose ethers such as methylcellulose (MC), hydroxyethylcellulose (HEC), hydroxypropyl cellulose (HPC), hydroxypropyl methyl cellulose (HPMC), methylhydroxyethylcellulose (MHEC), methylhydroxypropylcellulose (MHPC), carboxymethyl cellulose (CMC) and its various salts, including, e.g., the sodium salt, hydroxyethylcarboxymethylcellulose (HECMC) and its various salts, carboxymethylhydroxyethylcellulose (CMHEC) and its various salts, other polysaccharides and polysaccharide derivatives such as starch, dextran, dextran derivatives, chitosan, and alginic acid and its various salts, carageenan, various gums, including xanthan gum, guar gum, gum arabic, gum karaya, gum ghatti, konjac and gum tragacanth, glycosaminoglycans and proteoglycans such as hyaluronic acid and its salts, proteins such as gelatin, collagen, albumin, and fibrin, other polymers, for example, polyhydroxyacids such as polylactide, polyglycolide, polyl(lactide-co-glycolide) and poly(.epsilon.-caprolactone-co-glycolide)-, carboxyvinyl polymers and their salts (e.g., carbomer), polyvinylpyrrolidone (PVP), polyacrylic acid and its salts, polyacrylamide, polyacrylic acid/acrylamide copolymer, polyalkylene oxides such as polyethylene oxide, polypropylene oxide, poly(ethylene oxide-propylene oxide), and a Pluronic polymer, polyoxy ethylene (polyethylene glycol), polyanhydrides, polyvinylalchol, polyethyleneamine and polypyrridine, polyethylene glycol (PEG) polymers, such as PEGylated lipids (e.g., PEG-stearate, 1,2-Distearoyl-sn-glycero-3-Phosphoethanolamine-N-[Methoxy(Polyethylene glycol)-1000], 1,2-Distearoyl-sn-glycero-3-Phosphoethanolamine-N-[Methoxy(Polyethylene glycol)-2000], and 1,2-Distearoyl-sn-glycero-3-Phosphoethanolamine-N-[Methoxy(Polyethylene glycol)-5000]), copolymers and salts thereof.

Additionally, the composition may further comprise an emulsifying agent. Exemplary emulsifying agents include, but are not limited to, a polyethylene glycol (PEG), a polypropylene glycol, a polyvinyl alcohol, a poly-N-vinyl pyrrolidone and copolymers thereof, poloxamer nonionic surfactants, neutral water-soluble polysaccharides (e.g., dextran, Ficoll, celluloses), non-cationic poly(meth)acrylates, non-cationic polyacrylates, such as poly (meth) acrylic acid, and esters amide and hydroxy alkyl amides thereof, natural emulsifiers (e.g. acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g. bentonite [aluminum silicate] and Veegum [magnesium aluminum silicate]), long chain amino acid derivatives, high molecular weight alcohols (e.g. stearyl alcohol, cetyl alcohol, oleyl alcohol, triacetin monostearate, ethylene glycol distearate, glyceryl monostearate, and propylene glycol monostearate, polyvinyl alcohol), carbomers (e.g. carboxy polymethylene, polyacrylic acid, acrylic acid polymer, and carboxy vinyl polymer), carrageenan, cellulosic derivatives (e.g. carboxymethylcellulose sodium, powdered cellulose, hydroxymethyl cellulose, hydroxypropyl cellulose, hydroxypropyl methylcellulose, methylcellulose), sorbitan fatty acid esters (e.g. polyoxyethylene sorbitan monolaurate [Tween 20], polyoxyethylene sorbitan [Tween 60], polyoxyethylene sorbitan monooleate [Tween 80], sorbitan monopalmitate [Span 40], sorbitan monostearate [Span 60], sorbitan tristearate [Span 65], glyceryl monooleate, sorbitan monooleate [Span 80]), polyoxyethylene esters (e.g. polyoxyethylene monostearate [Myrj 45], polyoxyethylene hydrogenated castor oil, polyethoxylated castor oil, polyoxymethylene stearate, and Solutol), sucrose fatty acid esters, polyethylene glycol fatty acid esters (e.g. Cremophor), polyoxyethylene ethers, (e.g. polyoxyethylene lauryl ether [Brij 30]), poly(vinyl-pyrrolidone), diethylene glycol monolaurate, triethanolamine oleate, sodium oleate, potassium oleate, ethyl oleate, oleic acid, ethyl laurate, sodium lauryl sulfate, Pluronic F 68, Poloxamer 188, cetrimonium bromide, cetylpyridinium chloride, benzalkonium chloride, docusate sodium, etc. and/or combinations thereof. In certain embodiments, the emulsifying agent is cholesterol.

Liquid compositions include emulsions, microemulsions, solutions, suspensions, syrups, and elixirs. In addition to the active compound, the liquid composition may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable compositions, for example, injectable aqueous or oleaginous suspensions may be formulated according to the known art using suitable dispersing or wetting agents and suspending agents. The sterile injectable preparation may also be a injectable solution, suspension, or emulsion in a nontoxic parenterally acceptable diluent or solvent, for example, as a solution in 1,3-butanediol. Among the acceptable vehicles and solvents for pharmaceutical or cosmetic compositions that may be employed are water, Ringer's solution, U.S.P. and isotonic sodium chloride solution. In addition, sterile, fixed oils are conventionally employed as a solvent or suspending medium. Any bland fixed oil can be employed including synthetic mono- or diglycerides. In addition, fatty acids such as oleic acid are used in the preparation of injectables. In certain embodiments, the particles are suspended in a carrier fluid comprising 1% (w/v) sodium carboxymethyl cellulose and 0.1% (v/v) Tween 80. The injectable composition can be sterilized, for example, by filtration through a bacteria-retaining filter, or by incorporating sterilizing agents in the form of sterile solid compositions which can be dissolved or dispersed in sterile water or other sterile injectable medium prior to use.

Compositions for rectal or vaginal administration may be in the form of suppositories which can be prepared by mixing the particles with suitable non-irritating excipients or carriers such as cocoa butter, polyethylene glycol, or a suppository wax which are solid at ambient temperature but liquid at body temperature and therefore melt in the rectum or vaginal cavity and release the particles.

Solid compositions include capsules, tablets, pills, powders, and granules. In such solid compositions, the particles are mixed with at least one excipient and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets, and pills, the dosage form may also comprise buffering agents. Solid compositions of a similar type may also be employed as fillers in soft and hard-filled gelatin capsules using such excipients as lactose or milk sugar as well as high molecular weight polyethylene glycols and the like.

Tablets, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions which can be used include polymeric substances and waxes. Solid compositions of a similar type may also be employed as fillers in soft and hard-filled gelatin capsules using such excipients as lactose or milk sugar as well as high molecular weight polyethylene glycols and the like.

Compositions for topical or transdermal administration include ointments, pastes, creams, lotions, gels, powders, solutions, sprays, inhalants, or patches. The active compound is admixed with an excipient and any needed preservatives or buffers as may be required.

The ointments, pastes, creams, and gels may contain, in addition to the active compound, excipients such as animal and vegetable fats, oils, waxes, paraffins, starch, tragacanth, cellulose derivatives, polyethylene glycols, silicones, bentonites, silicic acid, talc, and zinc oxide, or mixtures thereof.

Powders and sprays can contain, in addition to the active compound, excipients such as lactose, talc, silicic acid, aluminum hydroxide, calcium silicates, and polyamide powder, or mixtures of these substances. Sprays can additionally contain customary propellants such as chlorofluorohydrocarbons.

Transdermal patches have the added advantage of providing controlled delivery of a compound to the body. Such dosage forms can be made by dissolving or dispensing the nanoparticles in a proper medium. Absorption enhancers can also be used to increase the flux of the compound across the skin. The rate can be controlled by either providing a rate controlling membrane or by dispersing the particles in a polymer matrix or gel.

The active ingredient may be administered in such amounts, time, and route deemed necessary in order to achieve the desired result. The exact amount of the active ingredient will vary from subject to subject, depending on the species, age, and general condition of the subject, the severity of the medical disorder, the particular active ingredient, its mode of administration, its mode of activity, and the like. The active ingredient, whether the active compound itself, or the active compound in combination with an agent, is preferably formulated in dosage unit form for ease of administration and uniformity of dosage. It will be understood, however, that the total daily usage of the active ingredient will be decided by the attending physician within the scope of sound medical judgment. The specific therapeutically effective dose level for any particular subject will depend upon a variety of factors including the disorder being treated and the severity of the disorder; the activity of the active ingredient employed; the specific composition employed; the age, body weight, general health, sex and diet of the patient; the time of administration, route of administration, and rate of excretion of the specific active ingredient employed; the duration of the treatment; drugs used in combination or coincidental with the specific active ingredient employed; and like factors well known in the medical arts.

The active ingredient may be administered by any route. In some embodiments, the active ingredient is administered via a variety of routes, including oral, intravenous, intramuscular, intra-arterial, intramedullary, intrathecal, subcutaneous, intraventricular, transdermal, interdermal, rectal, intravaginal, intraperitoneal, topical (as by powders, ointments, creams, and/or drops), mucosal, nasal, bucal, enteral, sublingual; by intratracheal instillation, bronchial instillation, and/or inhalation; and/or as an oral spray, nasal spray, and/or aerosol. In general, the most appropriate route of administration will depend upon a variety of factors including the nature of the active ingredient (e.g., its stability in the environment of the gastrointestinal tract), the condition of the subject (e.g., whether the subject is able to tolerate oral administration), etc.

The exact amount of an active ingredient required to achieve a therapeutically or prophylactically effective amount will vary from subject to subject, depending on species, age, and general condition of a subject, severity of the side effects or disorder, identity of the particular compound(s), mode of administration, and the like. The amount to be administered to, for example, a child or an adolescent can be determined by a medical practitioner or person skilled in the art and can be lower or the same as that administered to an adult.

Useful dosages of the active agents and pharmaceutical compositions disclosed herein can be determined by comparing their in vitro activity, and in vivo activity in animal models. Methods for the extrapolation of effective dosages in mice, and other animals, to humans are known to the art.

The dosage ranges for the administration of the compositions are those large enough to produce the desired effect in which the symptoms or disorder are affected. The dosage should not be so large as to cause adverse side effects, such as unwanted cross-reactions, anaphylactic reactions, and the like. Generally, the dosage will vary with the age, condition, sex and extent of the disease in the patient and can be determined by one of skill in the art. The dosage can be adjusted by the individual physician in the event of any counterindications. Dosage can vary, and can be administered in one or more dose administrations daily, for one or several days.

### Methods of Use

In another aspect, the present disclosure provides methods for treating, inhibiting, decreasing, reducing, ameliorating and/or preventing an infection with a virus in a subject in need thereof, the method comprising administering a therapeutically effective amount of a compound described herein, or a pharmaceutically acceptable salt thereof. In another aspect, the present disclosure provides methods for treating, inhibiting, decreasing, reducing, ameliorating and/or preventing a disease and/or symptoms associated with an infection with a virus in a subject in need thereof, comprising administering a therapeutically effective amount of a compound described herein, or a pharmaceutically acceptable salt or derivative thereof.

Viruses that are suitable for the methods and uses described herein can include both DNA viruses and RNA viruses. Exemplary viruses can belong to the following none exclusive list of families Adenoviridae, Arenaviridae, Astroviridae, Baculoviridae, Barnaviridae, Betaherpesvirinae, Bimaviridae, Bromoviridae, Bunyaviridae, Caliciviridae, Chordopoxvirinae, Circoviridae, Comoviridae, Coronaviridae, Cystoviridae, Corticoviridae, Entomopoxvirinae, Filoviridae, Flaviviridae, Fuselloviridae, Geminiviridae, Hepadnaviridae, Herpesviridae, Gammaherpesvirinae, Inoviridae, Iridoviridae, Leviviridae, Lipothrixviridae, Microviridae, Myoviridae, Nodaviridae, Orthomyxoviridae, Papovaviridae, Paramyxoviridae, Paramyxovirinae, Partitiviridae, Parvoviridae, Phycodnaviridae, Picornaviridae, Plasmaviridae, Pneumovirinae, Podoviridae, Polydnaviridae, Potyviridae, Poxviridae, Reoviridae, Retroviridae, Rhabdoviridae, Sequiviridae, Siphoviridae, Tectiviridae, Tetraviridae, Togaviridae, Tombusviridae, and Totiviridae.

Specific examples of suitable viruses include, but are not limited to, Mastadenovirus, Human adenovirus 2, Aviadenovirus, African swine fever virus, arenavirus, Lymphocytic choriomeningitis virus, Ippy virus, Lassa virus, Arterivirus, Human astrovirus 1, Nucleopolyhedrovirus, Autographa californica nucleopolyhedrovirus, Granulovirus, Plodia interpunctella granulovirus, Badnavirus, Commelina yellow mottle virus, Rice tungro bacilliform, Barnavirus, Mushroom bacilliform virus, Aquabirnavirus, Infectious pancreatic necrosis virus, Avibimavirus, Infectious bursal disease virus, Entomobimavirus, Drosophila X virus, Alfamovirus, Alfalfa mosaic virus, Ilarvirus, Ilarvirus Subgroups 1-10, Tobacco streak virus, Bromovirus, Brome mosaic virus, Cucumovirus, Cucumber mosaic virus, Bhanja virus Group, Kaisodi virus, Mapputta virus, Okola virus, Resistencia virus, Upolu virus, Yogue virus, Bunyavirus, Anopheles A virus, Anopheles B virus, Bakau virus, Bunyamwera virus, Bwamba virus, C virus, California encephalitis virus, Capim virus, Gamboa virus, Guama virus, Koongol virus, Minatitlan virus, Nyando virus, Olifantsvlei virus, Patois virus, Simbu virus, Tete virus, Turlock virus, Hantavirus, Hantaan virus, Nairovirus, Crimean-Congo hemorrhagic fever virus, Dera Ghazi Khan virus, Hughes virus, Nairobi sheep disease virus, Qalyub virus, Sakhalin virus, Thiafora virus, Crimean-congo hemorrhagic fever virus, Phlebovirus, Sandfly fever virus, Bujaru complex, Candiru complex, Chilibre complex, Frijoles complex, Punta Toro complex, Rift Valley fever complex, Salehabad complex, Sandfly fever Sicilian virus, Uukuniemi virus, Uukuniemi virus, Tospovirus, Tomato spotted wilt virus, Calicivirus, Vesicular exanthema of swine virus, Capillovirus, Apple stem grooving virus, Carlavirus, Carnation latent virus, Caulimovirus, Cauliflower mosaic virus, Circovirus, Chicken anemia virus, Closterovirus, Beet yellows virus, Comovirus, Cowpea mosaic virus, Fabavirus, Broad bean wilt virus 1, Nepovirus, Tobacco ringspot virus, Coronavirus, Avian infectious bronchitis virus, Bovine coronavirus, Canine coronavirus, Feline infectious peritonitis virus, Human coronavirus 299E, Human coronavirus OC43, Murine hepatitis virus, Porcine epidemic diarrhea virus, Porcine hemagglutinating encephalomyelitis virus, Porcine transmissible gastroenteritis virus, Rat coronavirus, Turkey coronavirus, Rabbit coronavirus, Torovirus, Berne virus, Breda virus, Corticovirus, Alteromonas phage PM2, Pseudomonas Phage phi6, Deltavirus, Hepatitis delta virus, Dianthovirus Carnation ringspot virus, Red clover necrotic mosaic virus, Sweet clover necrotic mosaic virus, Enamovirus, Pea enation mosaic virus, Filovirus, Marburg virus, Ebola virus Zaire, Flavivirus, Yellow fever virus, Tick-borne encephalitis virus, Rio Bravo Group, Japanese encephalitis, Tyuleniy Group, Ntaya Group, Uganda S Group, Dengue Group, Modoc Group, Pestivirus, Bovine diarrhea virus, Hepatitis C virus, Furovirus, Soil-borne wheat mosaic virus, Beet necrotic yellow vein virus, Fusellovirus, Sulfobolus virus 1, Subgroup I, II, and III geminivirus, Maize streak virus, Beet curly top virus, Bean golden mosaic virus, Orthohepadnavirus, Hepatitis B virus, Avihepadnavirus, Alphaherpesvirinae, Simplexvirus, Human herpesvirus 1, Varicellovirus, Human herpesvirus 3, Cytomegalovirus, Human herpesvirus 5, Muromegalovirus, Mouse cytomegalovirus 1, Roseolovirus, Human herpesvirus 6, Lymphocryptovirus, Human herpesvirus 4, Rhadinovirus, Ateline herpesvirus 2, Hordeivirus, Barley stripe mosaic virus, Hypoviridae, Hypovirus, Cryphonectria hypovirus 1-EP713, Idaeovirus, Raspberry bushy dwarf virus, Inovirus, Coliphage fd, Plectrovirus, Acholeplasma phage L51, Iridovirus, Chilo iridescent virus, Chloriridovirus, Mosquito iridescent virus, Ranavirus, Frog virus 3, Lymphocystivirus, Lymphocystis disease virus flounder isolate, Goldfish virus 1, Levivirus, Enterobacteria phage MS2, Allolevirus, Enterobacteria phage Qbeta, Lipothrixvirus, Thermoproteus virus 1, Luteovirus, Barley yellow dwarf virus, Machlomovirus, Maize chlorotic mottle virus, Marafivirus, Maize rayado fino virus, Microvirus, Coliphage phiX174, Spiromicrovirus, Spiroplasma phage 4, Bdellomicrovirus, Bdellovibrio phage MAC 1, Chlamydiamicrovirus, Chlamydia phage 1, T4-like phages, coliphage T4, Necrovirus, Tobacco necrosis virus, Nodavirus, Nodamura virus, Influenzavirus A, B and C, Thogoto virus, Polyomavirus, Murine polyomavirus, Papillomavirus, Rabbit (Shope) Papillomavirus, Paramyxovirus, Human parainfluenza virus 1, Morbillivirus, Measles virus, Rubulavirus, Mumps virus, Pneumovirus, Human respiratory syncytial virus, Partitivirus, Gaeumannomyces graminis virus 019/6-A, Chrysovirus, Penicillium chrysogenum virus, Alphacryptovirus, White clover cryptic viruses 1 and 2, Betacryptovirus, Parvovirinae, Parvovirus, Minute mice virus, Erythrovirus, B19 virus, Dependovirus, Adeno-associated virus 1, Densovirinae, Densovirus, Junonia coenia densovirus, Iteravirus, Bombyx mori virus, Contravirus, Aedes aegypti densovirus, Phycodnavirus, 1-Paramecium bursaria Chlorella NC64A virus group, Paramecium bursaria chlorella virus 1, 2-Paramecium bursaria Chlorella Pbi virus, 3-Hydra viridis Chlorella virus, Enterovirus, Human poliovirus 1, Rhinovirus Human rhinovirus 1A, Hepatovirus, Human hepatitis A virus, Cardiovirus, Encephalomyocarditis virus, Aphthovirus, Foot-and-mouth disease virus, Plasmavirus Acholeplasma phage L2, Podovirus, Coliphage T7, Ichnovirus, Campoletis sonorensis virus, Bracovirus, Cotesia melanoscela virus, Potexvirus, Potato virus X, Potyvirus, Potato virus Y, Rymovirus, Ryegrass mosaic virus, Bymovirus, Barley yellow mosaic virus, Orthopoxvirus, Vaccinia virus, Parapoxvirus, Orf virus, Avipoxvirus, Fowlpox virus, Capripoxvirus, Sheep pox virus, Leporipoxvirus, Myxoma virus, Suipoxvirus, Swinepox virus, Molluscipoxvirus, Molluscum contagiosum virus, Yatapoxvirus, Yaba monkey tumor virus, Entomopoxviruses A, B, and C, Melolontha melolontha entomopoxvirus, Amsacta moorei entomopoxvirus, Chironomus luridus entomopoxvirus, Orthoreovirus, Mammalian orthoreoviruses, reovirus 3, Avian orthoreoviruses, Orbivirus, African horse sickness viruses 1, Bluetongue viruses 1, Changuinola virus, Corriparta virus, Epizootic hemarrhogic disease virus 1, Equine encephalosis virus, Eubenangee virus group, Lebombo virus, Orungo virus, Palyam virus, Umatilla virus, Wallal virus, Warrego virus, Kemerovo virus, Rotavirus, Groups A-F rotaviruses, Simian rotavirus SA11, Coltivirus, Colorado tick fever virus, Aquareovirus, Groups A-E aquareoviruses, Golden shiner virus, Cypovirus, Cypovirus types 1-12, Bombyx mori cypovirus 1, Fijivirus, Fijivirus groups 1-3, Fiji disease virus, Fijivirus groups 2-3, Phytoreovirus, Wound tumor virus, Oryzavirus, Rice ragged stunt, Mammalian type B retroviruses, Mouse mammary tumor virus, Mammalian type C retroviruses, Murine Leukemia Virus, Reptilian type C oncovirus, Viper retrovirus, Reticuloendotheliosis virus, Avian type C retroviruses, Avian leukosis virus, Type D Retroviruses, Mason-Pfizer monkey virus, BLV-HTLV retroviruses, Bovine leukemia virus, Lentivirus, Bovine lentivirus, Bovine immunodeficiency virus, Equine lentivirus, Equine infectious anemia virus, Feline lentivirus, Feline immunodeficiency virus, Canine immunodeficiency virus Ovine/caprine lentivirus, Caprine arthritis encephalitis virus, Visna/maedi virus, Primate lentivirus group, Human immunodeficiency virus 1, Human immunodeficiency virus 2, Human immunodeficiency virus 3, Simian immunodeficiency virus, Spumavirus, Human spuma virus, Vesiculovirus, Vesicular stomatitis Indiana virus, Lyssavirus, Rabies virus, Ephemerovirus, Bovine ephemeral fever virus, Cytorhabdovirus, Lettuce necrotic yellows virus, Nucleorhabdovirus, Potato yellow dwarf virus, Rhizidiovirus, Rhizidiomyces virus, Sequivirus, Parsnip yellow fleck virus, Waikavirus, Rice tungro spherical virus, Lambda-like phages, Coliphage lambda, Sobemovirus, Southern bean mosaic virus, Tectivirus, Enterobacteria phage PRD1, Tenuivirus, Rice stripe virus, Nudaurelia capensis beta-like viruses, Nudaurelia beta virus, Nudaurelia capensis omega-like viruses, Nudaurelia omega virus, Tobamovirus, Tobacco mosaic virus (vulgare strain; ssp. NC82 strain), Tobravirus, Tobacco rattle virus, Alphavirus, Sindbis virus, Rubivirus, Rubella virus, Tombusvirus, Tomato bushy stunt, virus, Carmovirus, Carnation mottle virus, Turnip crinkle virus, Totivirus, Saccharomyces cerevisiae virus, Giardiavirus, Giardia lamblia virus, Leishmaniavirus, Leishmania brasiliensis virus 1-1, Trichovirus, Apple chlorotic leaf spot virus, Tymovirus, Turnip yellow mosaic virus, Umbravirus, and Carrot mottle virus.

The present disclosure also provides methods for treating, inhibiting, decreasing, reducing, ameliorating and/or preventing a coronavirus infection in a subject in need thereof, the method comprising administering a therapeutically effective amount of a compound described herein, or a pharmaceutically acceptable salt thereof. In another aspect, the present disclosure provides methods for treating, inhibiting, decreasing, reducing, ameliorating and/or preventing the disease and/or symptoms associated with a coronavirus infection in a subject in need thereof, comprising administering a therapeutically effective amount of a compound described herein, or a pharmaceutically acceptable salt or derivative thereof. A “coronavirus infection” as used herein refers to an infection caused by or otherwise associated with growth of coronavirus in a subject, in the family Coronaviridae (subfamily Coronavirinae).

In one aspect, a method is provided for treating a coronavirus infection in a subject in need thereof, the method comprising administering a therapeutically effective amount of a compound described herein, or a pharmaceutically acceptable salt thereof.

In another aspect, a method is provided for treating a disease associated with a coronavirus infection in a subject in need thereof, the method comprising administering a therapeutically effective amount of a compound described herein, or a pharmaceutically acceptable salt thereof.

In another aspect, a method is provided for treating, inhibiting, decreasing, reducing, ameliorating and/or preventing one or more symptoms associated with a coronavirus infection in a subject in need thereof, the method comprising administering a therapeutically effective amount of a compound described herein, or a pharmaceutically acceptable salt thereof.

Coronaviruses are species of virus belonging to the subfamily Coronavirinae in the family Coronaviridae, in the order Nidovirales. Coronaviruses are enveloped viruses with a positive-sense single-stranded RNA genome and with a nucleocapsid of helical symmetry.

The genomic size of coronaviruses ranges from approximately 26 to 32 kilobases, the largest for an RNA virus. The name “coronavirus” is derived from the Latin corona, meaning crown or halo, and refers to the characteristic appearance of virions under electron microscopy (E.M.) with a fringe of large, bulbous surface projections creating an image reminiscent of a royal crown or of the solar corona. This morphology is created by the viral spike (S) peplomers, which are proteins that populate the surface of the virus and determine host tropism. Proteins that contribute to the overall structure of all coronaviruses are the spike (S), envelope (E), membrane (M) and nucleocapsid (N) proteins. In the specific case of the SARS coronavirus and SARS coronavirus 2, a defined receptor-binding domain on S mediates the attachment of the virus to its cellular receptor, angiotensin-converting enzyme 2 (ACE2). Some coronaviruses (specifically the members of Betacoronavirus subgroup A) also have a shorter spike-like protein called hemagglutinin esterase (HE).

In one embodiment, the coronavirus infection is an infection of the upper and/or lower respiratory tract. The “upper respiratory tract” includes the mouth, nose, sinus, middle ear, throat, larynx, and trachea. The “lower respiratory tract” includes the bronchial tubes (bronchi) and the lungs (bronchi, bronchioles and alveoli), as well as the interstitial tissue of the lungs.

In another embodiment, the coronavirus infection is an infection of the gastrointestinal tract. The “gastrointestinal tract” may include any area of the canal from the mouth to the anus, including the mouth, esophagus, stomach, and intestines.

In yet another embodiments, the coronavirus infection is a renal infection.

It is understood and herein contemplated that the coronavirus infections disclosed herein can cause a pathological state associated with the coronavirus infection referred to herein as a “coronavirus disease.” In some embodiments, the coronavirus disease is selected from a common cold, pneumonia, pneumonitis, bronchitis, severe acute respiratory syndrome (SARS), coronavirus disease 2019 (COVID-2019), Middle East respiratory syndrome (MERS), sinusitis, porcine diarrhea, porcine epidemic diarrhea, avian infectious bronchitis, otitis and pharyngitis. In particular embodiments, the coronavirus disease is a common cold. In particular embodiments, the coronavirus disease is selected from SARS, COVID-19, and MERS. In a particular embodiment, the coronavirus disease is COVID-19. In another particular embodiment, the coronavirus disease is IBV, PorCoV HKU15, or PEDV.

Other indications associated with coronavirus infections are described in Gralinski & Baric, 2015, J. Pathol. 235:185-195 and Cavanagh, 2005, “Coronaviridae: a review of coronavirus and toroviruses”, Coronaviruses with Special Emphasis on First Insights Concerning SARS 1, ed. By A. Schmidt, M. H. Wolff and O. Weber, Birkhauser Verlag Baser, Switzerland, each of which is incorporated herein by reference in their entirety.

The coronavirus causing the infection may be selected from an alphacoronavirus, a betacoronavirus, a gammacoronavirus, or a deltacoronavirus.

Representative examples of alphacoronaviruses include, but are not limited to, a colacovirus (e.g., Bat coronavirus CDPHE15), a decacovirus (e.g., Bat coronavirus HKU10, Rhinolophus ferrumequinum alphacoronavirus Hub-2013), a duvinacovirus (e.g., Human coronavirus 229E), a luchacovirus (e.g., Lucheng Rn rat coronavirus), a minacovirus (e.g., Ferret coronavirus, Mink coronavirus 1), a minunacovirus (e.g., Miniopterus bat coronavirus 1, Miniopterus bat coronavirus HKU8), a myotacovirus (e.g., Myotis rickettii alphacoronavirus Sax-2011), a nyctacovirus (e.g., Nyctalus velutinus alphacoronavirus SC-2013), a pedacovirus (e.g., Porcine epidemic diarrhea virus (PEDV), Scotophilus bat coronavirus 512), a rhinacovirus (e.g., Rhinolophus bat coronavirus HKU2), a setracovirus (e.g., Human coronavirus NL63, NL63-related bat coronavirus strain BtKYNL63-9b), or a tegacovirus (e.g. Alphacoronavirus 1).

Representative examples of betacoronaviruses include, but are not limited to an embecovirus 1 (e.g., Betacoronavirus 1, Human coronavirus OC43, China Rattus coronavirus HKU24, Human coronavirus HKU1, Murine coronavirus), a hibecovirus (e.g., Bat Hp-betacoronavirus Zhejiang2013), a merbecovirus (e.g., Hedgehog coronavirus 1, Middle East respiratory syndrome-related coronavirus (MERS-CoV), Pipistrellus bat coronavirus HKU5, Tylonycteris bat coronavirus HKU4), a nobecovirus (e.g., Rousettus bat coronavirus GCCDC1, Rousettus bat coronavirus HKU9), or a sarbecovirus (e.g., severe acute respiratory syndrome coronavirus (SARS-CoV), severe acute respiratory syndrome coronavirus 2 (SARS-CoV-2).

Representative examples of gammacoronaviruses include, but are not limited to, a cegacovirus (e.g., Beluga whale coronavirus SQ1) or an Igacovirus (e.g., Avian coronavirus (IBV)).

Representative examples of deltacoronaviruses include, but are not limited to, an andecovirus (e.g., Wigeon coronavirus HKU20), a buldecovirus (e.g., Bulbul coronavirus HKU11, Porcine coronavirus HKU15 (PorCoV HKU15), Munia coronavirus HKU13, White-eye coronavirus HKU16), a herdecovirus (e.g., Night heron coronavirus HKU19), or a moordecovirus (e.g., Common moorhen coronavirus HKU21).

In some embodiments, the coronavirus is a human coronavirus. Representative examples of human coronaviruses include, but are not limited to, human coronavirus 229E (HCoV-229E), human coronavirus OC43 (HCoV-OC43), human coronavirus HKU1 (HCoV-HKU1), Human coronavirus NL63 (HCoV-NL63), severe acute respiratory syndrome coronavirus (SARS-CoV), severe acute respiratory syndrome coronavirus 2 (SARS-CoV-2), and Middle East respiratory syndrome-related coronavirus (MERS-CoV).

In one embodiment, a method is provided for treating coronavirus disease 2019 (COVID-2019) comprising administering a therapeutically effective amount of a compound described herein, or a pharmaceutically acceptable salt thereof.

In one embodiment, a method is provided for preventing coronavirus disease 2019 (COVID-2019) comprising administering a therapeutically effective amount of a compound described herein, or a pharmaceutically acceptable salt thereof.

In another embodiment, a method is provided for inhibiting, decreasing, reducing, ameliorating and/or preventing one or more symptoms associated with coronavirus disease 2019 (COVID-2019) comprising administering a therapeutically effective amount of a compound described herein, or a pharmaceutically acceptable salt thereof.

In another aspect, a method is provided for inhibiting replication of a coronavirus in a coronavirus-infected cell, the method comprising contacting the cell with a therapeutically effective amount of a compound described herein, or a pharmaceutically acceptable salt thereof. In some embodiments, the coronavirus is SARS-CoV-2. In some embodiments, the cell is a human cell.

In another aspect, a method is provided for reducing viral load of a coronavirus in a coronavirus-infected cell, the method comprising contacting the cell with a therapeutically effective amount of compound described herein, or a pharmaceutically acceptable salt thereof. In some embodiments, the coronavirus is SARS-CoV-2. In some embodiments, the cell is a human cell.

In some embodiments, the compounds as used in the methods described herein may be administered in combination or alternation with one or more additional active agents. Representative examples additional active agents include antimicrobial agents (including antibiotics, antiviral agents and anti-fungal agents), anti-inflammatory agents (including steroids and non-steroidal anti-inflammatory agents) and antiseptic agents.

Representative examples of antibiotics include amikacin, amoxicillin, ampicillin, atovaquone, azithromycin, aztreonam, bacitracin, carbenicillin, cefadroxil, cefazolin, cefdinir, cefditoren, cefepime, cefiderocol, cefoperazone, cefotetan, cefoxitin, cefotaxime, cefpodoxime, cefprozil, ceftaroline, ceftazidime, ceftibuten, ceftizoxime, ceftriaxone, chloramphenicol, colistimethate, cefuroxime, cephalexin, cephradine, cilastatin, cinoxacin, ciprofloxacin, clarithromycin, clindamycin, dalbavancin, dalfopristin, daptomycin, demeclocycline, dicloxacillin, doripenem, doxycycline, eravacycline, ertapenem, erythromycin, fidaxomicin, fosfomycin, gatifloxacin, gemifloxacin, gentamicin, imipenem, lefamulin, lincomycin, linezolid, lomefloxacin, loracarbef, meropenem, metronidazole, minocycline, moxifloxacin, nafcillin, nalidixic acid, neomycin, norfloxacin, ofloxacin, omadacycline, oritavancin, oxacillin, oxytetracycline, paromomycin, penicillin, pentamidine, piperacillin, plazomicin, quinupristin, rifaximin, sarecycline, secnidazole, sparfloxacin, spectinomycin, sulfamethoxazole, sulfisoxazole, tedizolid, telavancin, telithromycin, ticarcillin, tigecycline, tobramycin, trimethoprim, trovafloxacin, and vancomycin.

Representative examples of antiviral agents include, but are not limited to, abacavir, acyclovir, adefovir, amantadine, amprenavir, atazanavir, balavir, baloxavir marboxil, boceprevir, cidofovir, cobicistat, daclatasvir, darunavir, delavirdine, didanosine, docasanol, dolutegravir, doravirine, ecoliever, edoxudine, efavirenz, elvitegravir, emtricitabine, enfuvirtide, entecavir, etravirine, famciclovir, fomivirsen, fosamprenavir, forscarnet, fosnonet, famciclovir, favipravir, fomivirsen, foscavir, ganciclovir, ibacitabine, idoxuridine, indinavir, inosine, inosine pranobex, interferon type I, interferon type II, interferon type III, lamivudine, letermovir, letermovir, lopinavir, loviride, maraviroc, methisazone, moroxydine, nelfinavir, nevirapine, nitazoxanide, oseltamivir, peginterferon alfa-2a, peginterferon alfa-2b, penciclovir, peramivir, pleconaril, podophyllotoxin, pyramidine, raltegravir, remdesevir, ribavirin, rilpivirine, rimantadine, rintatolimod, ritonavir, saquinavir, simeprevir, sofosbuvir, stavudine, tarabivirin, telaprevir, telbivudine, tenofovir alafenamide, tenofovir disoproxil, tenofovir, tipranavir, trifluridine, trizivir, tromantadine, umifenovir, valaciclovir, valganciclovir, vidarabine, zalcitabine, zanamivir, and zidovudine.

Representative examples of antifungal agents include, but are not limited to, voriconazole, itraconazole, posaconazole, fluconazole, ketoconazole, clotrimazole, isavuconazonium, miconazole, caspofungin, anidulafungin, micafungin, griseofulvin, terbinafine, flucytosine, terbinafine, nystatin, and amphotericin b.

Representative examples of steroidal anti-inflammatory agents include, but are not limited to, hydrocortisone, dexamethasone, prednisolone, prednisone, triamcinolone, methylprednisolone, budesonide, betamethasone, cortisone, and deflazacort. Representative examples of non-steroidal anti-inflammatory drugs include ibuprofen, naproxen, ketoprofen, tolmetin, etodolac, fenoprofen, flurbiprofen, diclofenac, piroxicam, indomethacin, sulindax, meloxicam, nabumetone, oxaprozin, mefenamic acid, and diflunisal.

### Additional Aspects of the Disclosure

The following description includes information that may be useful in understanding the present invention. It is not an admission that any of the information provided herein is prior art or relevant to the presently claimed invention, or that any publication specifically or implicitly referenced is prior art.

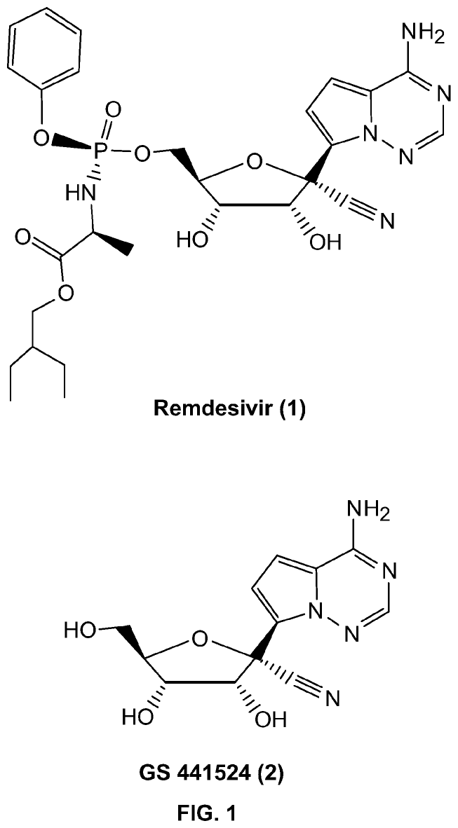

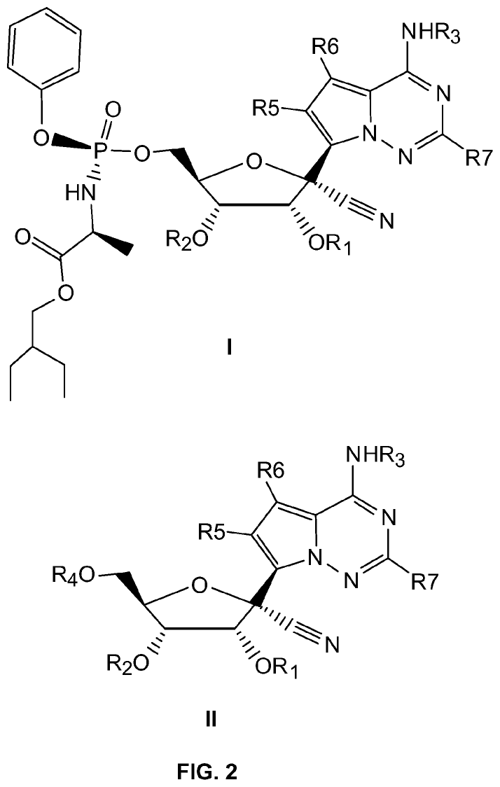

Inventors have found that fatty acyl conjugates of remdesivir and its active analogs or metabolite, such as GS 441524 (2, FIG. 1), have activity against SARS-COV-2 and other viruses that are dependent on RdRp for replication. The fatty acylated conjugates cross the cell membrane, achieving higher concentrations in target cells followed by released parent compound endow the conjugates with dual-action antiviral activity. The development of viral resistance to the parent drug would occur at a slower rate.

This conjugation with fatty acids results in the development of antiviral agents, such as anti-SARS-COV-2 compounds having efficacy against SARS-COV2, Ebola, and other coronaviruses, longer duration of action by the sustained intracellular release of active substrates at adequate concentrations, and higher uptake into infected cells.

Various objects, features, aspects and advantages of the inventive subject matter will become more apparent from the following detailed description of preferred embodiments, along with the accompanying drawing figures in which like numerals represent like components.

One should appreciate that the disclosed techniques provide many advantageous technical effects, including providing safe and effective means for treating coronavirus infections that also reduce the likelihood of the development of drug-resistant viral strains.

The following discussion provides many example embodiments of the inventive subject matter. Although each embodiment represents a single combination of inventive elements, the inventive subject matter is considered to include all possible combinations of the disclosed elements. Thus if one embodiment comprises elements A, B, and C, and a second embodiment comprises elements B and D, then the inventive subject matter is also considered to include other remaining combinations of A, B, C, or D, even if not explicitly disclosed.

As used herein, and unless the context dictates otherwise, the term “coupled to” is intended to include both direct coupling (in which two elements that are coupled to each other contact each other) and indirect coupling (in which at least one additional element is located between the two elements). Therefore, the terms “coupled to” and “coupled with” are used synonymously.

Disclosed herein are compounds, compositions, methods for making and using such compounds and compositions. In specific aspects, disclosed are novel fatty acid-nucleoside and fatty ether systems containing remdesivir (1) or its parent analog or metabolite GS 441524 (2, (2R,3R,4S,5R)-2-(4-aminopyrrolo[2,1-f][1,2,4]triazin-7-yl)-3,4-dihydroxy-5-(hydroxymethyl)tetrahydrofuran-2-carbonitrile) and natural and unnatural fatty acid analogs, which can be used as antiviral agents, including anti-coronavirus agents.

The rationale of this design is based on the data that fatty acylation of nucleosides generates prodrug conjugates with comparable or enhanced efficacy, longer duration of action by the sustained intracellular release of active substrates at adequate concentrations, and higher uptake into infected cells. The released fatty acids and nucleosides will act by different mechanisms. The development of viral resistance to the parent drug would occur at a slower rate.

The present invention relates to fatty acid and fatty alcohol substituted nucleoside derivatives of remdesivir (1) and GS 441524 (2) that display potent antiviral activity.

The present invention relates to novel nucleoside derivatives and nucleoside conjugates. The compounds and compositions of the invention may be used systemically as therapeutic or preventative agents.

These fatty acids or fatty alcohols may be connected in different ways to remdesivir, active analog, its metabolite, or active analogs through attachment to free hydroxyl or amino groups or any combination of them or through a linker. One or more than one fatty acids or fatty alcohols can be connected either to two hydroxyl groups, one hydroxyl/one amino group or all free amino and hydroxyl groups directly or through a linker.

The nucleoside backbone of remdesivir or its metabolite may, in exemplary aspects, be modified forms with substitutions at nucleoside base, and substitutions at positions 1′, 2′, 3′, 4′, and 5′ of carbohydrate moiety (Formulas I and II) and/or double bond between C3′ and C4′ in carbohydrate moiety or other nucleoside derivatives known to those skilled in the art while conjugated with fatty acids.

In another aspect, the fatty acid in Formulas I-II may be of the general formula X(CH₂)ₙY(CH₂)ₙCOOH or CH₃(CH₂)ₙCH(Br)COOH and the fatty alcohol is X(CH₂)ₙY(CH₂)ₙCH₂OH or CH₃(CH₂)ₙCH(Br)CH₂OH, wherein n=0-21; X=CH₃, N₃, alkyl-S, alkyl-O, aryl-O, aryl-S, alkyl-NH, aryl-NH, Br, Cl, F, I, OH, NH₂, COOH, CHO, CH₃S, aryl, heteroaryl, phenyl, alkene, alkyne, or substituted phenyl; and Y=CH₂, 0, S, NH, or 1,2,3-triazole.

In another aspect, the scaffold in Formulas I-II may be derivatives containing one to three 2-hydroxybenzyl alcohol (e.g., 4,4′-dihydroxy-3,3′-di-(hydroxymethyl)diphenylmethane, 4,6-dihydroxy-1,3-benzenedimethanol, 4,4′,4″-methanetriyltris(2-(hydroxymethyl)phenol)), polycarboxylic acids (e.g. tribenzenetriacetic acid, hydroxybenzendicarboxylic acid, [(hydroxyphenylene)dixoy]diacetic acid, tris(carboxymethoxy)benzene, and triazine-tricarboxylic acid derivatives, such as (triazinetriyltroxy)triacetic acid and 1,3,5-triazine-2,4,6-tricarboxylic acid), and anionic polymers (cellulose sulfate, cellulose sulfate acetate, dextran sulfate, naphthalene sulfonate derivatives, polystyrene sulfonate, carrageenans, polycarboxylic acid, or polyvinylpyrrolidone, and where other polyanionic compounds are polyphosphorylated polymers, suramin, cyclodextrin sulfate, or multisulfated and multiphosphorylated peptides and alkyl chains).

In another aspect, the compounds of Formulas I-II display antiviral activity against coronaviruses or other viruses.

In another aspect, Remdesivir or its active metabolite is acylated on free NH₂ and OH groups, in the chemical structure using fatty acids, such as 12-azidododecanoic acid, 12-thioethydodecanoic acid, tetradecanoic acid, and 4-oxatetradecanoic acid, fatty acyl chlorides, or fatty acid anhydrides in the presence of HBTU, DIC, and DIPEA as coupling and activating reagents, respectively, or other coupling and activating reagents using appropriate protection and deprotection steps.

In another aspect, the mono fatty acyl or fatty ether conjugates at 3′-, 4′-, or 5′-hydroxyl groups in Formulas I or II are synthesized by the methods described here or those known skills in art.

In another aspect, difatty or trifatty acyl or ether conjugates at 3′-, 4′-, and 5′-hydroxyl groups in Formulas I or II are synthesized by the methods described here or those known skills in art.

In another aspect, fatty acyl amide or alkyl amino-substituted conjugates in Formulas I or II are synthesized by the methods described here or those known skills in art.

In another aspect, a combination of multiple fatty acyl, fatty ether, and fatty acyl amides in any of free hydroxyl or free amino-substituted by the methods described here or those known skills in art.

In addition to direct ester or ether derivatives, the fatty acids or fatty alcohols are linked to the nucleosides through other linkers and/or scaffolds, including phosphoramidate, phosphotriesters, phosphodiesters, phosphomonoesters, triglycerides, linear peptide backbones, cyclic peptide backbone, hydroxyphenyldicarboxylic acid derivatives, and compounds containing multi cycloSaligenyl groups.

Linear and cyclic peptides can have glutamic acid or aspartic acid for the attachment of nucleosides and serine or threonine for the attachment of fatty acids. The number of amino acids can be of the desired length, preferably 1-25. Amino acids can be L or D. Polycarboxylic acid derivatives used as scaffolds for nucleosides conjugation include tribenzenetriacetic acid, hydroxybenzendicarboxylic acid, [(hydroxyphenylene)dixoy]diacetic acid, tris(carboxymethoxy)benzene, (triazinetriyltroxy)triacetic acid, triazine-tricarboxylic acid derivatives, such as (triazinetriyltroxy)triacetic acid and 1,3,5-triazine-2,4,6-tricarboxylic acid. Scaffolds containing one to three 2-hydroxybenzyl alcohol are conjugated through a phosphotriester (cyclosaligenyl phosphotriester) to nucleosides and/or fatty alcohols.

In another aspect, the compounds of Formulas I-II, may be chemically linked to another compound directly or through a linker, wherein the other compound is another nucleoside, a polymer, or a polyanionic molecule, wherein this compound is cellulose sulfate, cellulose sulfate acetate, dextran sulfate, naphthalene sulfonate derivatives, polystyrene sulfonate, carrageenans, polycarboxylic acid, polyvinylpyrrolidone, or cyclodextrin sulfate, and where other polyanionic compounds are polyphosphorylated polymers, suramin, or multisulfated and multiphosphorylated peptides and alkyl chains.

In another aspect, the compounds of Formulas I-II or one or more nucleoside or nucleotide analogs may be linked to a scaffold directly or through a linker in the presence or absence of fatty acids or fatty alcohols.

Synthesized compounds are active against SARS-CoV-2 and other coronaviruses and have potential activity as antiviral agents.

In one aspect, the compounds of Formulas I-II may be in the form of a composition which comprises a carrier, additive, or excipient.

In another aspect, the compounds of Formulas I-II may be in the form of a composition that may be used to treat or prevent infection, transmission, or acquisition of COVID-19 and other coronaviruses-related diseases.

In another aspect, the compounds of Formulas I-II may be chemically linked to another compound to provide a composition of matter and may contain a carrier or excipient, and may be used in a method for treating, preventing, or reducing viral diseases by delivering the composition of matter in injectable, solid or semi-solid forms, such as a tablet, film, gel, cream, ointment, pessary, or the like.

In another aspect, the compounds of Formulas I-II may be chemically linked to another compound to provide a composition of matter and may contain a carrier or excipient, and may be used in a method for preventing or treating the viral infection as part of a combination product, wherein the other components of the product are other nucleosides, nucleotide, or protease inhibitors, or integrase inhibitors or entry/fusion inhibitors, or other viral inhibitors known to those skilled in the art.

The present invention can be understood more readily by reference to the following detailed description of the invention, and the Examples and Figures included therein.

Within the context of the application a nucleoside analog should be understood as a member of a group of synthetic compounds that are structurally similar to natural nucleosides and interfere with DNA or RNA synthesis. Similarly, a nucleotide analog should be understood as a member of a group of synthetic compounds that are structurally similar to natural nucleotides and interfere with DNA or RNA synthesis. A remdesivir analog should be understood as a member of a group of synthetic compounds that include a core structure of remdesivir (e.g. the nucleotide analog) with one or more substitutions, deletions, and/or additions to the cores structure that are not represented in the parent nucleoside analog. A fatty acid analog should be understood as a synthetic molecule that incorporates one or more hydrocarbon chains corresponding to a naturally occurring lipid and/or fatty acid.

Before the present compounds, compositions, articles, systems, devices, and/or methods are disclosed and described, it is to be understood that they are not limited to specific synthetic methods unless otherwise specified, or to particular reagents unless otherwise specified, as such may, of course, vary. It is also to be understood that the terminology used herein is for the purpose of describing particular aspects only and is not intended to be limiting. Although any methods and materials similar or equivalent to those described herein can be used in the practice or testing of the present invention, example methods and materials are now described.

The following discussion provides many example embodiments of the inventive subject matter. Although each embodiment represents a single combination of inventive elements, the inventive subject matter is considered to include all possible combinations of the disclosed elements. Thus, if one embodiment comprises elements A, B, and C, and a second embodiment comprises elements B and D, then the inventive subject matter is also considered to include other remaining combinations of A, B, C, or D, even if not explicitly disclosed.

In some embodiments, the numbers expressing quantities of ingredients, properties such as concentration, reaction conditions, and so forth, used to describe and claim certain embodiments of the invention are to be understood as being modified in some instances by the term “about.” Accordingly, in some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the invention are approximations, the numerical values set forth in the specific examples are reported as precisely as practicable. The numerical values presented in some embodiments of the invention may contain certain errors necessarily resulting from the standard deviation found in their respective testing measurements. [0048] As used in the description herein and throughout the claims that follow, the meaning of “a,” “an,” and “the” includes plural reference unless the context clearly dictates otherwise. Also, as used in the description herein, the meaning of “in” includes “in” and “on” unless the context clearly dictates otherwise.

Throughout the description and claims of this specification the word “comprise” and other forms of the word, such as “comprising” and “comprises,” means including but not limited to, and is not intended to exclude, for example, other additives, components, integers, or steps.

As used herein, the terms “optional” or “optionally” means that the subsequently described event or circumstance can or cannot occur and that the description includes instances where said event or circumstance occurs and instances where it does not.

The term “Formulas I and II” includes all the substitutions in the nucleoside bases and carbohydrate moiety.

The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the invention and does not pose a limitation on the scope of the invention otherwise claimed.

No language in the specification should be construed as indicating any non-claimed element essential to the practice of the invention.

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified, thus fulfilling the written description of all Markush groups used in the appended claims.

Disclosed are novel fatty acid-nucleoside and fatty ether systems containing remdesivir (1) or its parent analog or metabolite GS 441524 (2) and natural and unnatural fatty acid analogs, which can be used as antiviral agents, including anti-coronavirus agents.

The disclosed compositions can include fatty acids or fatty alcohols connected in different ways to remdesivir, active analog, or its metabolite through attachment to free hydroxyl or amino groups or any combination of the. One or more than one fatty acids or fatty alcohols can be connected either to two hydroxyl groups, one hydroxyl/one amino group, or all free amino and hydroxyl groups.

The Inventors designed, synthesized, and characterized by analytical methods, such as NMR and mass spectrometry.

The nucleoside backbone of remdesivir or its metabolite may, in exemplary aspects, be modified forms with substitutions at nucleoside base, and substitutions at positions 1′, 2′, 3′, 4′, and 5′ of carbohydrate moiety, and/or double bond between C3′ and C4′ in carbohydrate moiety or other nucleoside derivatives known to those skilled in the art while conjugated with fatty acids.

The fatty acid in Formulas I-II may be of the general formula X(CH₂)ₙY(CH₂)ₙCOOH or CH₃(CH₂)ₙCH(Br)COOH and the fatty alcohol is X(CH₂)ₙY(CH₂)ₙCH₂OH or CH₃(CH₂)ₙCH(Br)CH₂OH, wherein n=0-21; X=CH₃, N₃, alkyl-S, alkyl-O, aryl-O, aryl-S, alkyl-NH, aryl-NH, Br, Cl, F, I, OH, NH₂, COOH, CHO, CH₃S, aryl, heteroaryl, phenyl, alkene, alkyne, or substituted phenyl; and Y=CH₂, 0, S, NH, or 1,2,3-triazole.

The compounds of Formulas I-II can display antiviral activity against any virus. Examples of susceptible virus include enveloped viruses, such as coronaviruses, Ebola virus, and Zika virus. Contemplated coronaviruses include SARS coronaviruses (e.g. SARS-CoV-2) and MERS coronaviruses.

Remdesivir and its active metabolite are acylated on free the NH₂ and/or OH groups, in the chemical structure using fatty acids, such as 12-azidododecanoic acid, 12-thioethydodecanoic acid, tetradecanoic acid, and 4-oxatetradecanoic acid, fatty acyl chlorides, or fatty acid anhydrides in the presence of HBTU, DIC, and DIPEA as coupling and activating reagents, respectively, or other coupling and activating reagents using appropriate protection and deprotection steps. The fatty acyl or fatty ether substitutions can be mono, di, tri, or tetra substitution using any of the free hydroxyl or amino groups and can be synthesized by methods described here or through known skills in art.

The compounds in Formulas I-II can be used as long-acting antiviral agents by sustained releases of active substrates at adequate concentrations and higher uptake into infected cells.

Compounds of the inventive concept represent a new and novel class of antiviral agents. The structures of these series of compounds are different than those of current antiviral agents.

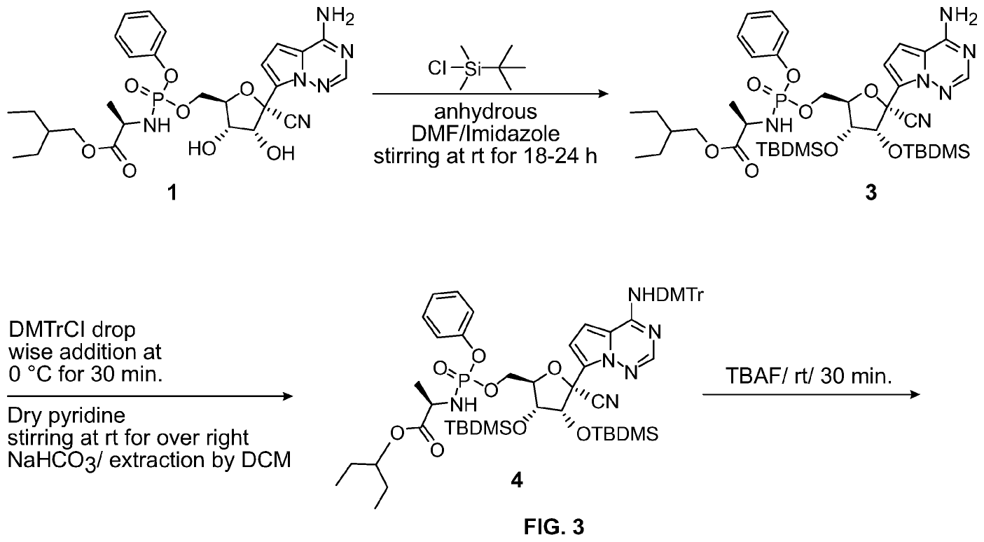

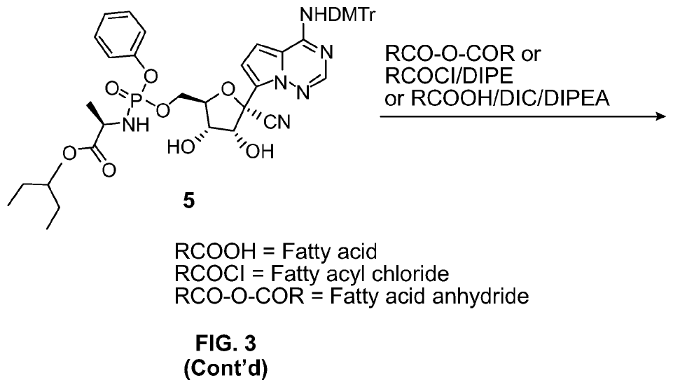

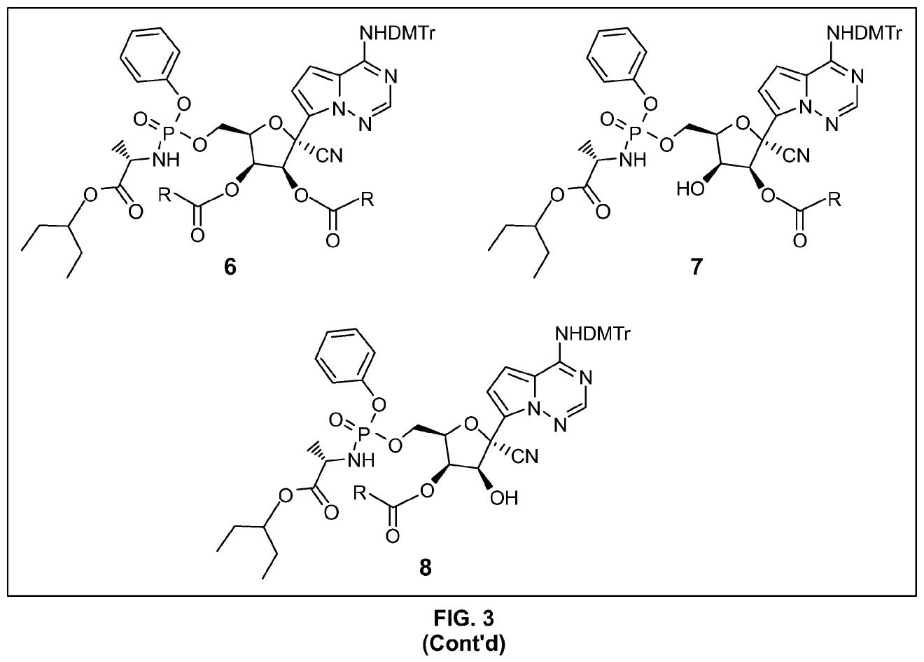

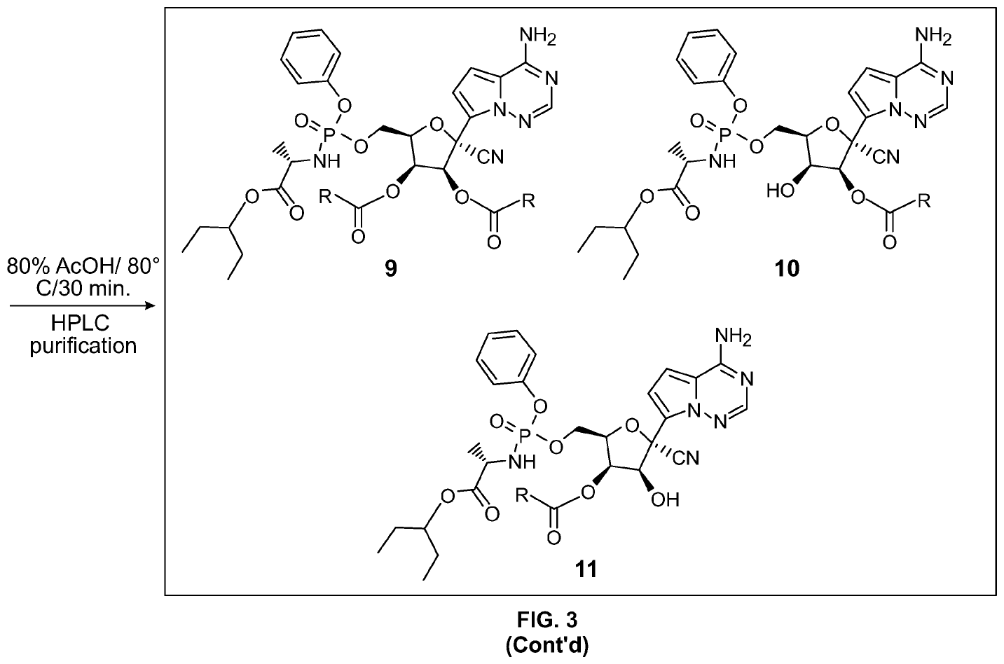

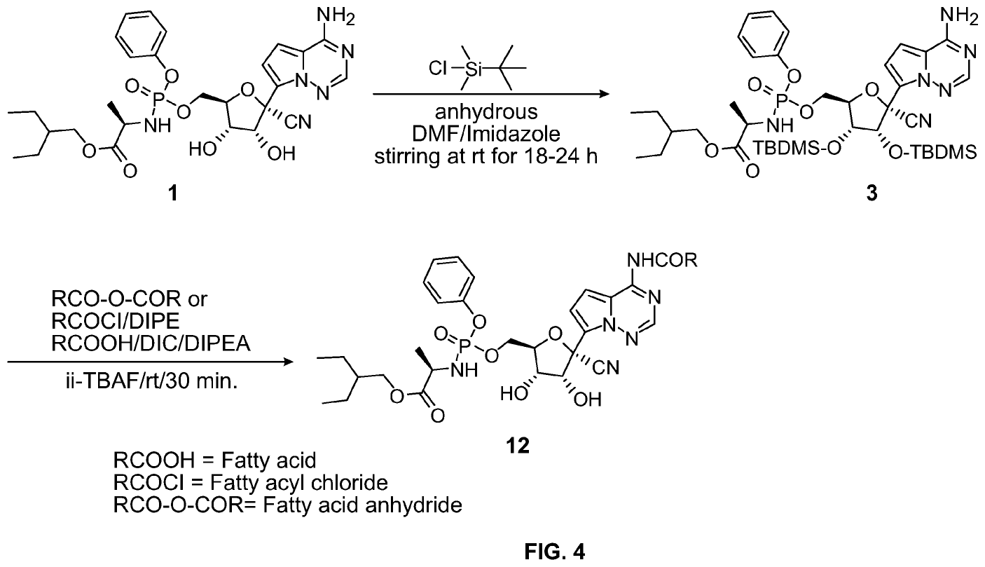

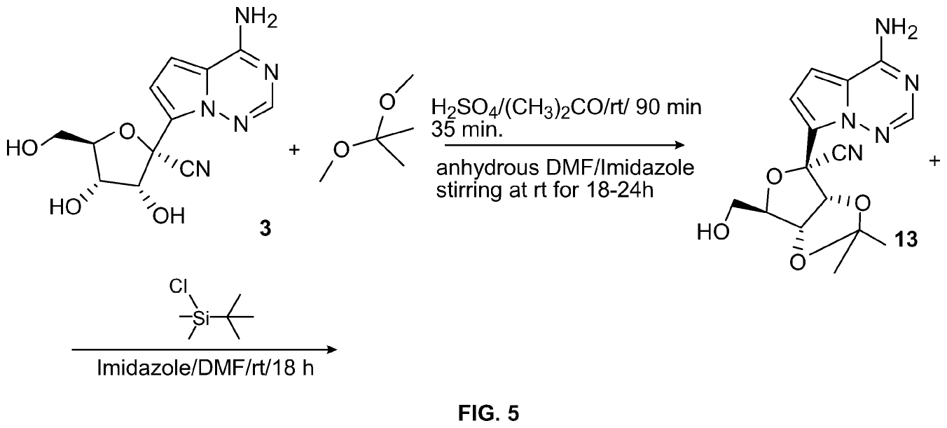

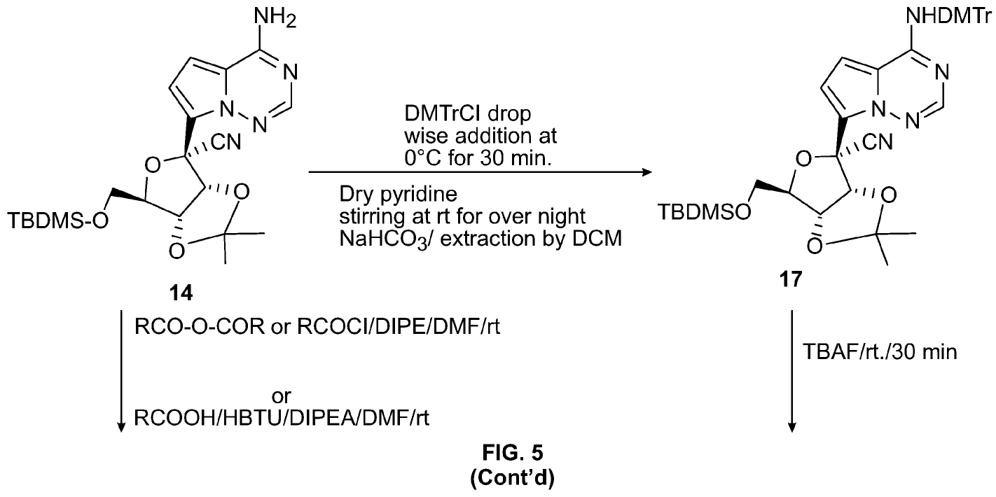

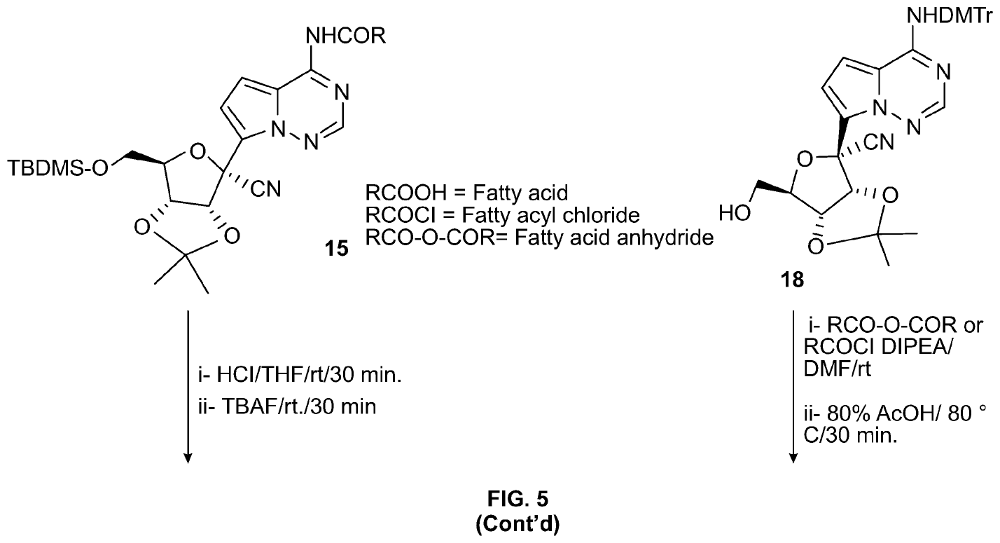

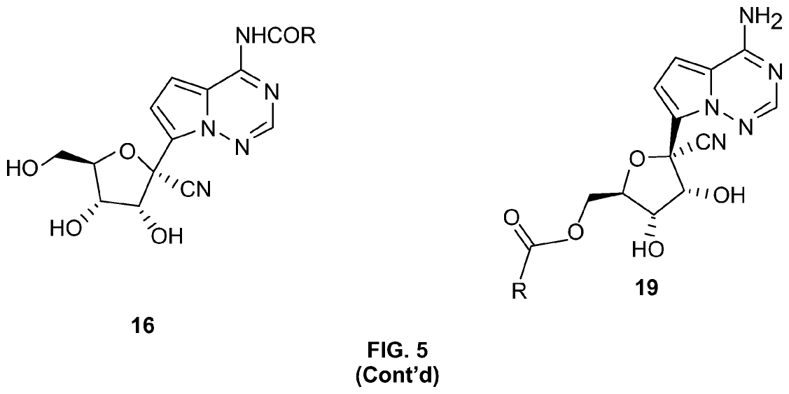

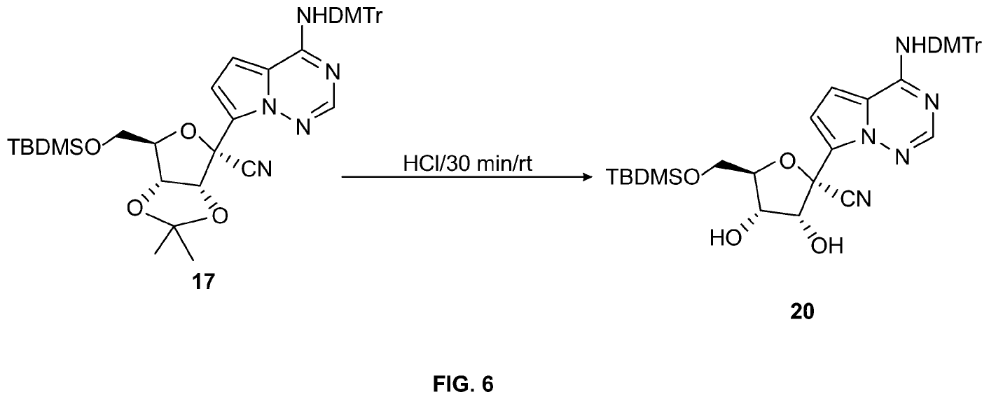

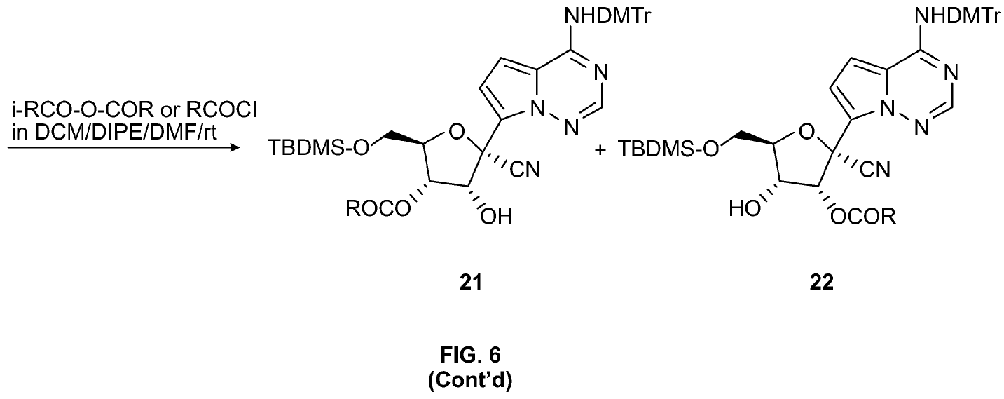

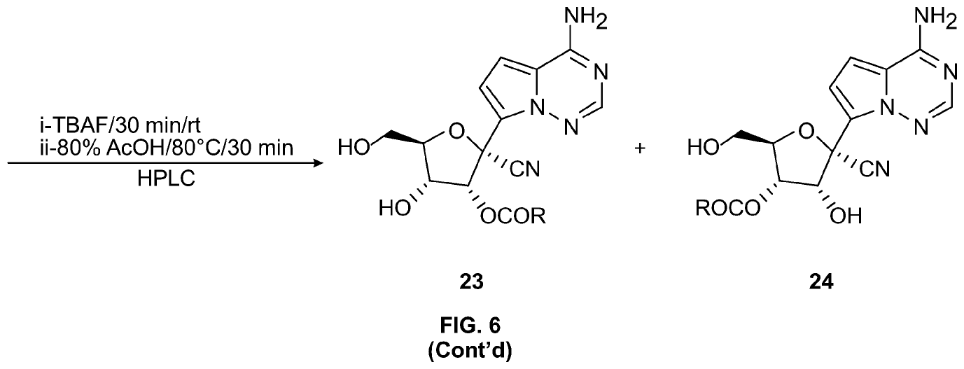

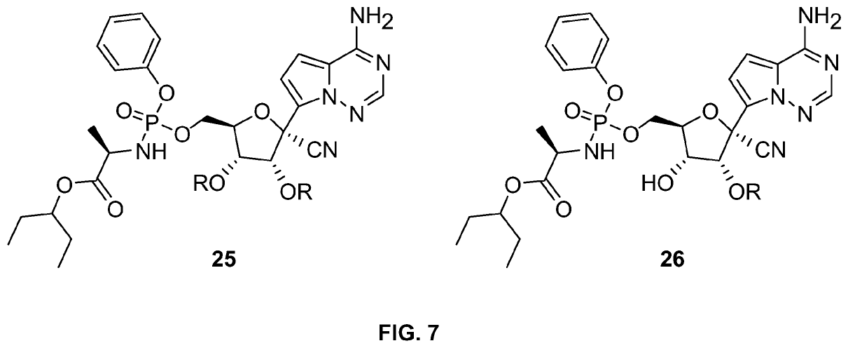

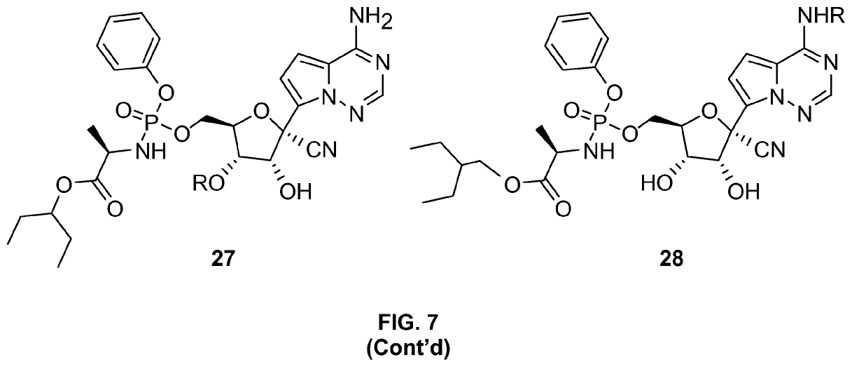

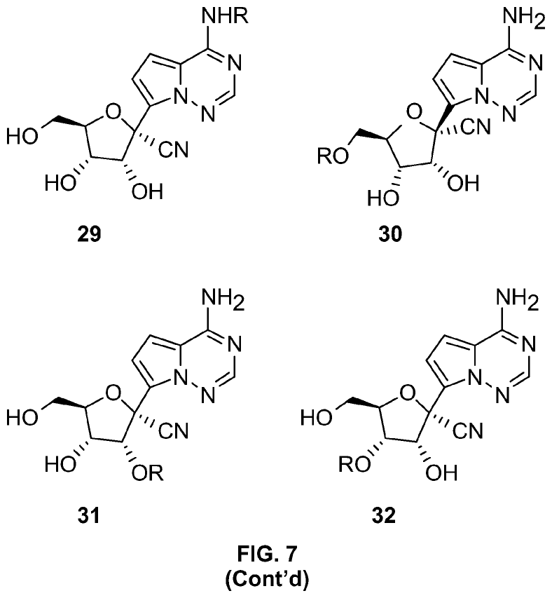

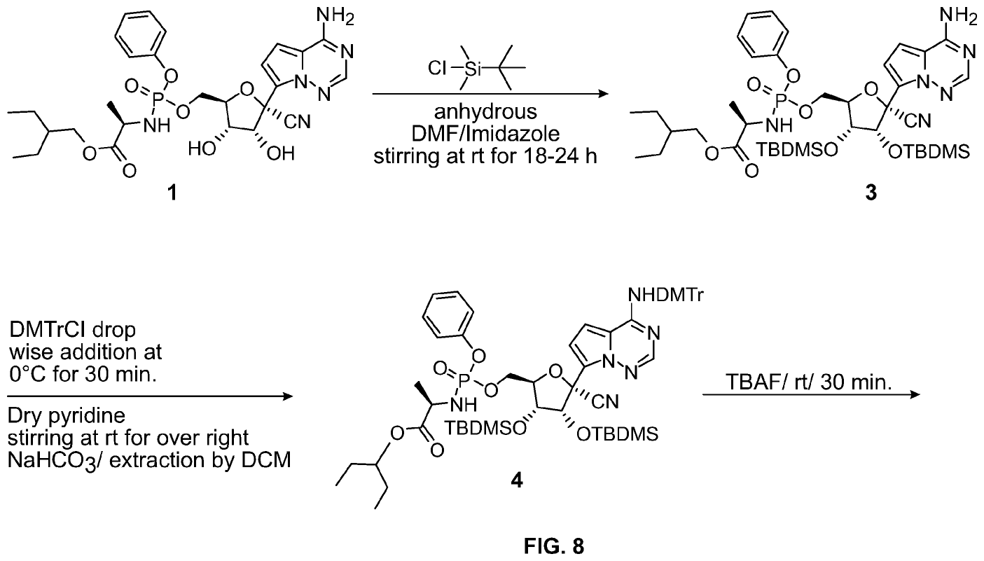

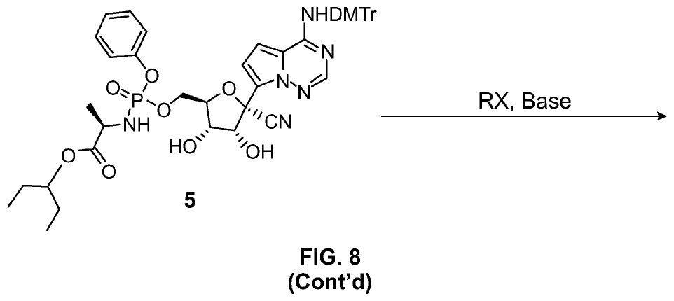

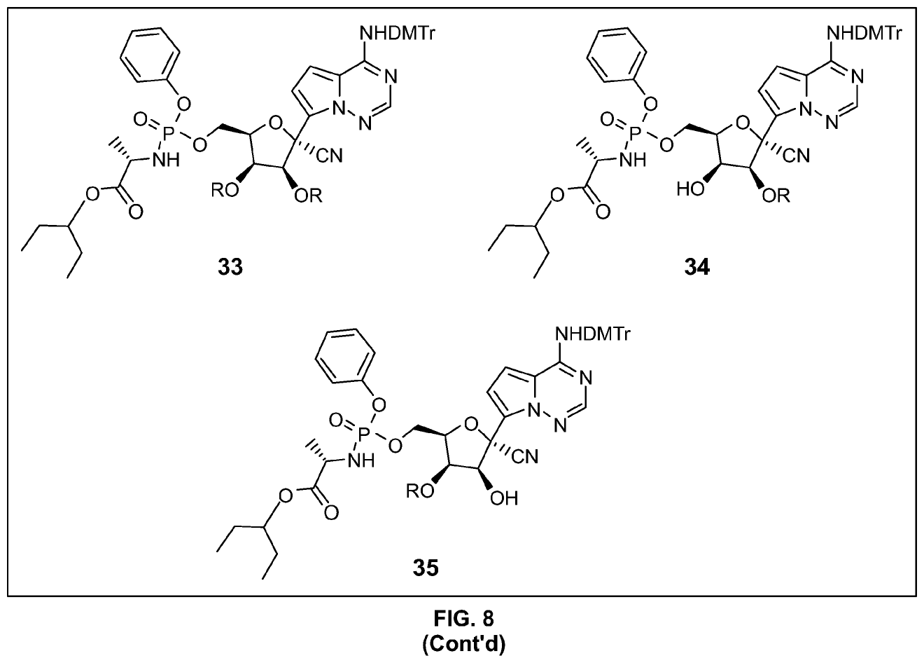

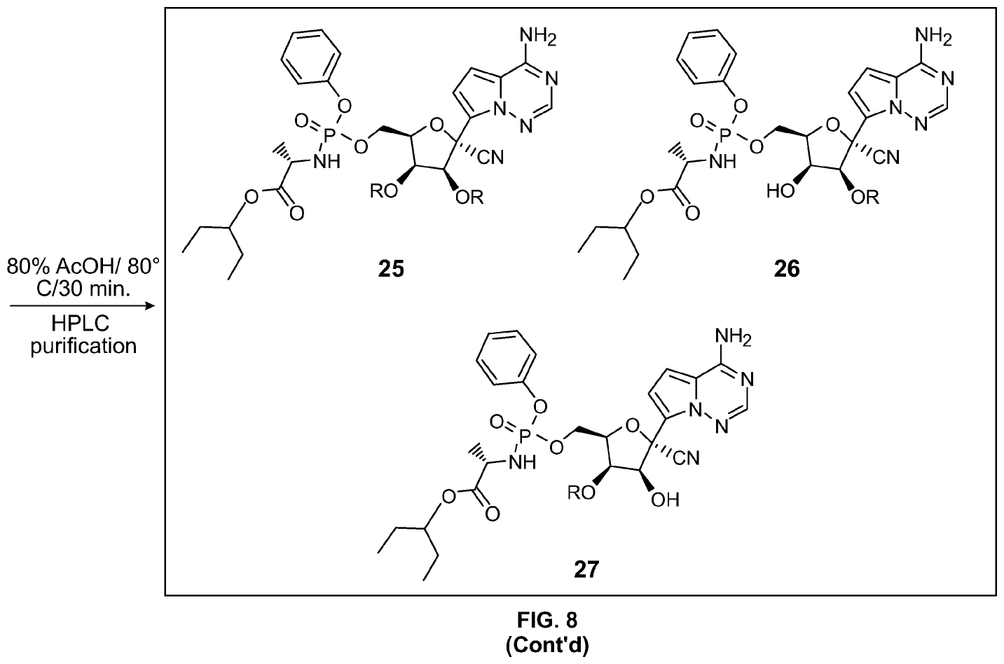

Examples of synthetic methodologies are shown in FIGS. 3 to 6.

Antiviral compounds were identified by slowing replication of the virus. As shown, compounds of the inventive concept significantly inhibit replication of these viruses and do so at concentrations that are physiologically achievable.

In consideration of their dual mode of action, Inventors believe that compounds of the inventive concept can exhibit antiviral activity against a broad range of viruses, in particular enveloped viruses. Examples of suitable DNA viruses include (but are not limited to) Herpesviruses, Poxviruses, Hepadnaviruses, and Asfarviridae. Suitable RNA viruses include (but are not limited to) Flavivirus, Alphavirus, Togavirus, Coronavirus, Hepatitis D, Orthomyxovirus, Paramyxovirus, Rhabdovirus, Bunyavirus, Filovirus, Retroviruses, and Retroviruses. In particular, the Applicant believes that compounds of the inventive concept can be effective against disease caused by a coronavirus, such as COVID-19.

Compounds of the inventive concept can be provided to an individual in need of treatment by any suitable route. Suitable routes include injection, infusion, topical application to skin, topical application to a mucus membrane (e.g. oral, nasal, vaginal, and/or rectal mucosa), application to the ocular surface, introduction to the gastrointestinal tract, and/or inhalation. Modes of application can vary depending on the viral disease being treated, the stage of the viral disease, and/or characteristics of the individual being treated. In some embodiments the manner of application of the drug can change over the course of treatment. For example, an individual presenting with acute symptoms may initially be treated by injection or infusion in order to rapidly provide useful concentrations of the drug, then moved to ingestion (for example, of a pill or tablet) to maintain such useful concentrations over time.

Accordingly, formulations that include a drug of the inventive concept can be provided in different forms and with different excipients. For example, formulations provided for ingestion can be provided as a liquid, a powder that is dissolved in a liquid prior to consumption, a pill, a tablet, or a capsule. Solid forms provided for ingestion can be provided with enteric coatings or similar features that provide release of the drug in a selected portion of the gastrointestinal tract (e.g. the small intestine) and/or provide sustained release of the drug over time. Formulations intended for topical application can be provided as a liquid, a gel, a paste, an ointment, and/or a powder. Such formulations can be provided as part of a dressing, film, or similar appliance that is placed on a body surface. Formulations intended for injection (e.g. subcutaneous, intramuscular, intraocular, intraperitoneal, intravenous, etc.) or infusion can be provided as a liquid or as a dry form (such as a powder) that is dissolved or suspended in liquid prior to use. Formulations intended for inhalation can similarly be provided in a liquid form or a dray form that is suspended or dissolved in liquid prior to use, or as a dry powder of particle size suitable for inhalation. Such inhaled formulations can be provided as an atomized spray or subjected to nebulization to generate a liquid droplet suspension in air or other suitable gas vehicle for inhalation.

Liquid formulations can be in the form of a solution, a suspension, a micellar suspension, and/or an emulsion. Similarly, dry or granular formulations can be provided as lyophilized or spray-dried particulates, which in some embodiments can be individually encapsulated.

Compounds of the inventive concept can be provided in any amount that provides a suitably effective antiviral effect. It should be appreciated that this can vary for a given compound depending upon the route of administration, the virus being treated, and the characteristics of the individual being treated. Suitable doses can range from 0.1 μg/kg to 100 mg/kg body weight, or from 0.01 μg/mL to 100 mg/mL w/w/concentration.

Dosing schedules applied to a compound of the inventive concept can vary depending upon the virus being treated, the mode of application, the severity of the disease state, and the characteristics of the individual. In some embodiments, application of the drug can be essentially constant, for example, through infusion, incorporation into ongoing intravenous therapy, and/or inhalation. In other embodiments, a compound of the inventive concept can be applied once. In still other embodiments, a compound of the inventive concept can be provided periodically over a suitable period of time. For example, a compound of the inventive concept can be provided every 2 hours, every 3 hours, every 4 hours, every 6 hours, every 8 hours, every 12 hours, daily, on alternating days, twice a week, weekly, every two weeks, monthly, every 2 months, every 3 months, every 6 months, or yearly.

As noted above, formulation, dose, and dosing schedule for a compound of the inventive concept can vary depending on the state of the viral disease. In some embodiments, such a compound can be provided to an individual in need of prophylactic treatment, for example, to an uninfected individual in order to prevent the establishment of infection by a virus following exposure. In other embodiments, a compound of the inventive concept can be provided to an individual who is infected with a virus but is asymptomatic. In still other concepts, a compound of the inventive concept can be provided to an individual that is infected with a virus and is symptomatic. As noted above, dosing, route, and dosing schedule of the compound can be adjusted as symptoms of an active viral infection change.

In some embodiments, a compound as described above can be used in combination with one or more other active companion compounds. Suitable companion compounds include antiviral compounds, antibacterial compounds, antifungal compounds, anti-inflammatory compounds, bronchodilators, and compounds that treat pain. The Inventor anticipates that synergistic (i.e. greater than additive effects) can result from such combinations in regard to antiviral effect, reduction in disease time course, reduction in severity of symptoms, and/or morbidity.

Similarly, in some embodiments, two or more compounds as described above can be used in combination. The Inventor anticipates that synergistic (i.e. greater than additive effects) can result from such combinations in regard to antiviral effect, reduction in disease time course, reduction in the severity of symptoms, and/or morbidity.

It should be apparent to those skilled in the art that many more modifications besides those already described are possible without departing from the inventive concepts herein. The inventive subject matter, therefore, is not to be restricted except in the spirit of the appended claims. Moreover, in interpreting both the specification and the claims, all terms should be interpreted in the broadest possible manner consistent with the context. In particular, the terms “comprises” and “including” should be interpreted as referring to elements, components, or steps in a non-exclusive manner, indicating that the referenced elements, components, or steps may be present, or utilized, or combined with other elements, components, or steps that are not expressly referenced. Where the specification claims refer to at least one of something selected from the group consisting of A, B, C . . . and N, the text should be interpreted as requiring only one element from the group, not A plus N, or B plus N, etc.

A number of embodiments of the disclosure have been described. Nevertheless, it will be understood that various modifications may be made without departing from the spirit and scope of the invention. Accordingly, other embodiments are within the scope of the following claims.

By way of non-limiting illustration, examples of certain embodiments of the present disclosure are given below.

### EXAMPLES

The following examples are put forth so as to provide those of ordinary skill in the art with a complete disclosure and description of how the compounds, compositions, articles, devices and/or methods claimed herein are made and evaluated, and are intended to be purely exemplary of the invention and are not intended to limit the scope of what the inventors regard as their invention. However, those of skill in the art should, in light of the present disclosure, appreciate that many changes can be made in the specific embodiments which are disclosed and still obtain a like or similar result without departing from the spirit and scope of the invention.

Efforts have been made to ensure accuracy with respect to numbers (e.g., amounts, temperature, etc.), but some errors and deviations should be accounted for. Unless indicated otherwise, parts are parts by weight, the temperature is in ° C. or is at ambient temperature, and pressure is at or near atmospheric.

Chemicals and solvents were procured from Sigma-Aldrich. The final compounds used in further studies were purified with silica gel chromatography or reversed-phase high-performance liquid chromatography. The chemical structure of intermediates and final products were characterized by NMR and high-resolution MALDI-TOF or QTOF from Bruker Inc.

### General Method for Synthesis of the Nucleoside Conjugates

FIG. 3 depicts a general synthetic route for synthesis of the antiviral compounds described above. In general, appropriate protecting groups are used in the synthesis of monosubstituted, disubstituted, and other substituted analogs for compounds described I Formulas I and II. After fatty acylation, the protecting groups were removed using appropriate deprotecting reagents (FIGS. 3-6), and the final products are purified and characterized.

Alternatively, the compounds in Formulas I and II were directly reacted with fatty acids, fatty alcohols, fatty acyl chlorides, fatty alkyl chlorides, or fatty acid anhydrides, and the final products were purified from each other using flash chromatography, silica gel column chromatography or HPLC.

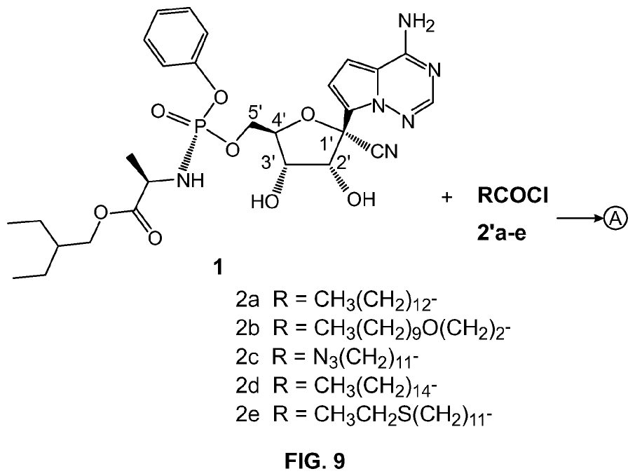

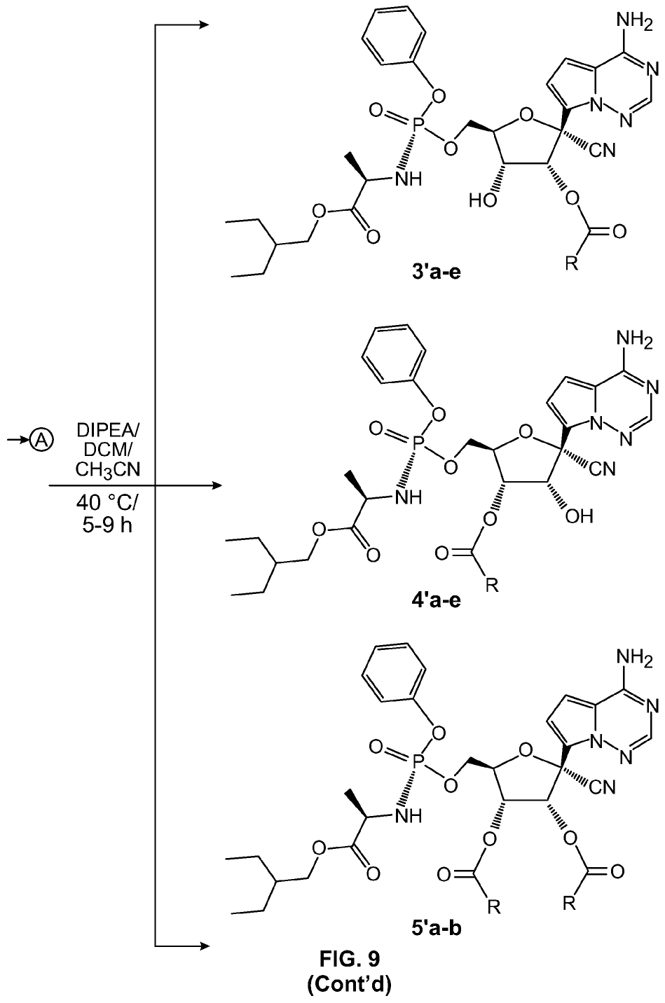

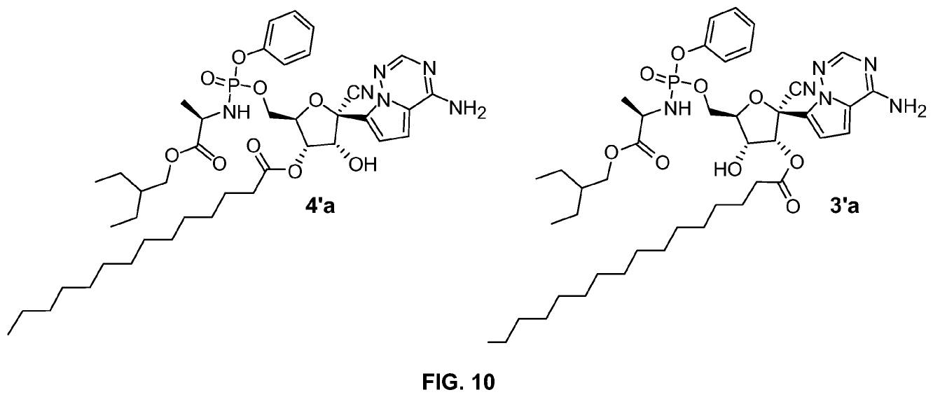

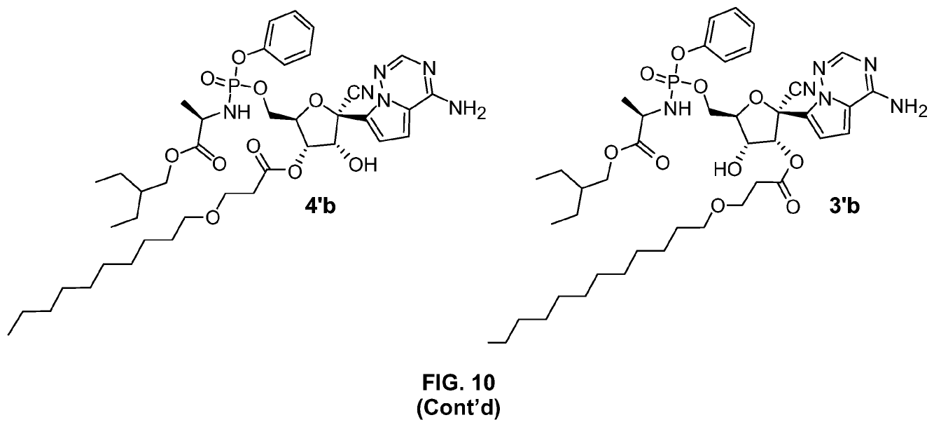

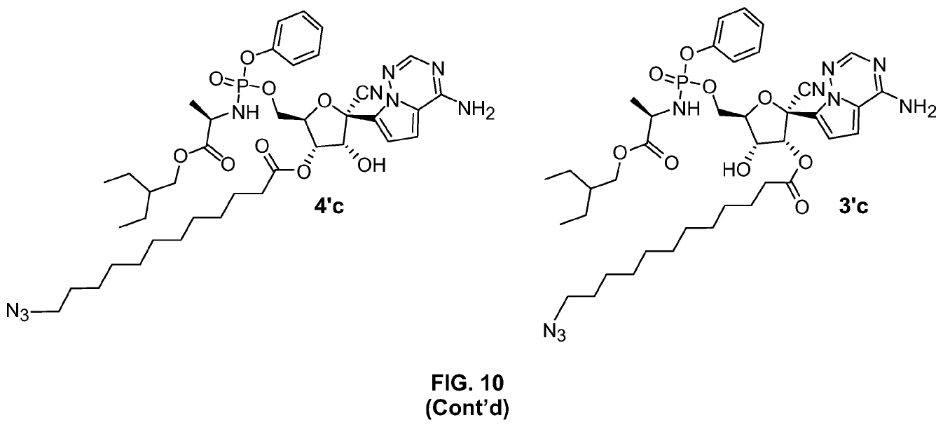

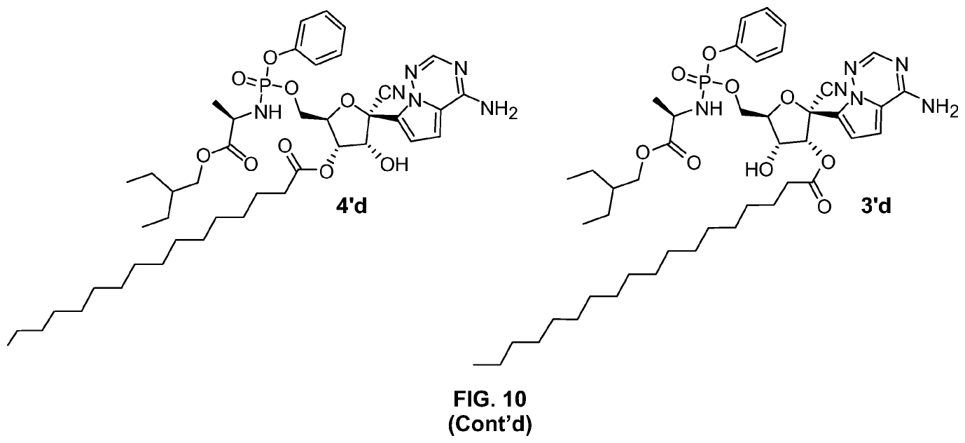

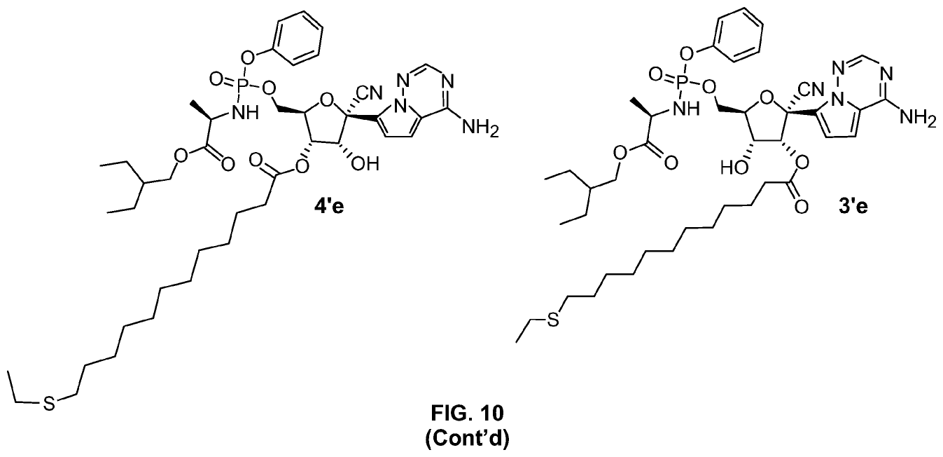

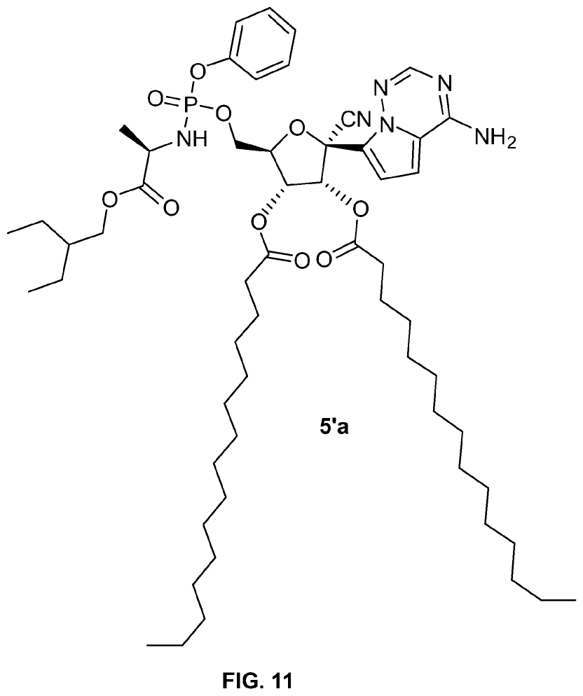

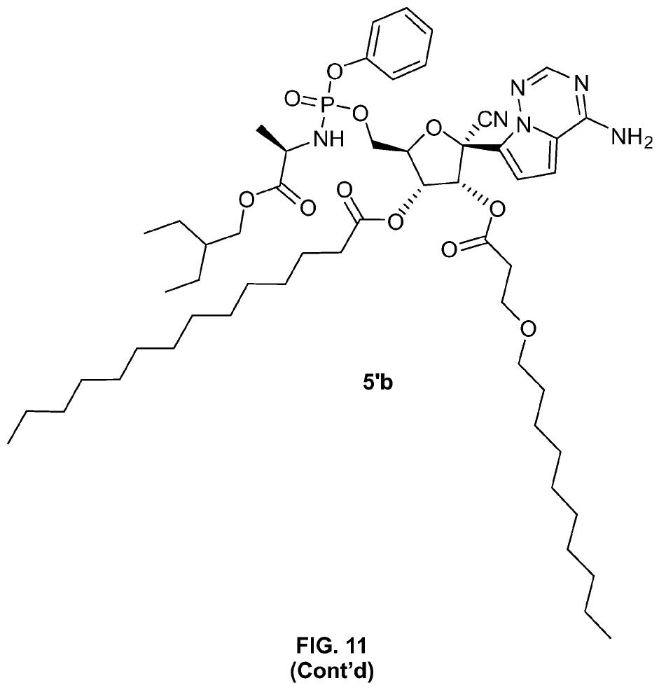

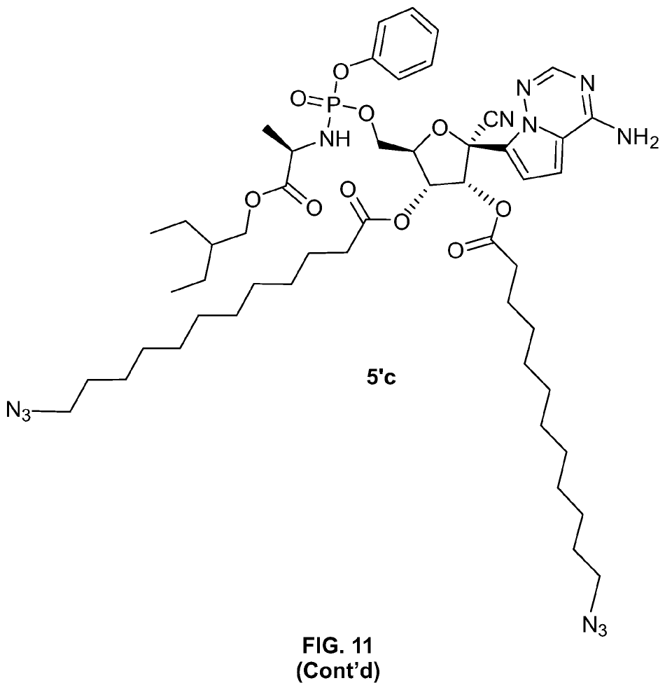

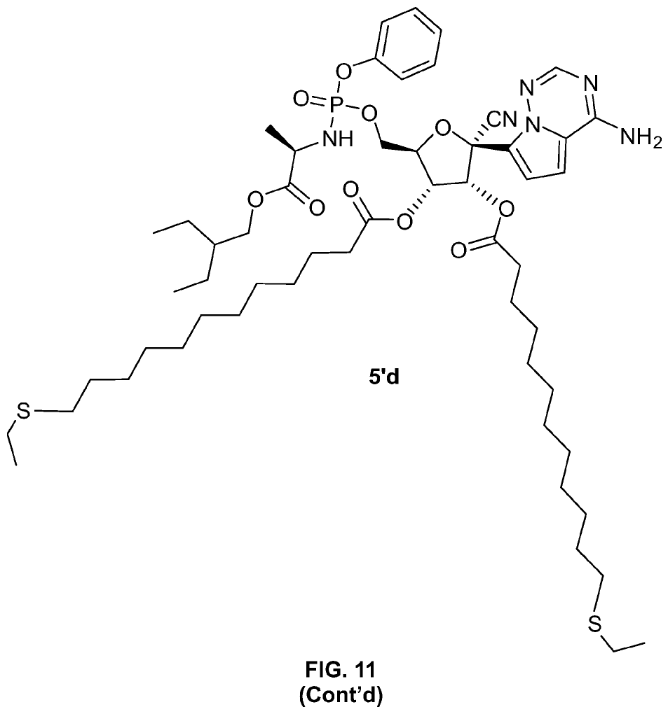

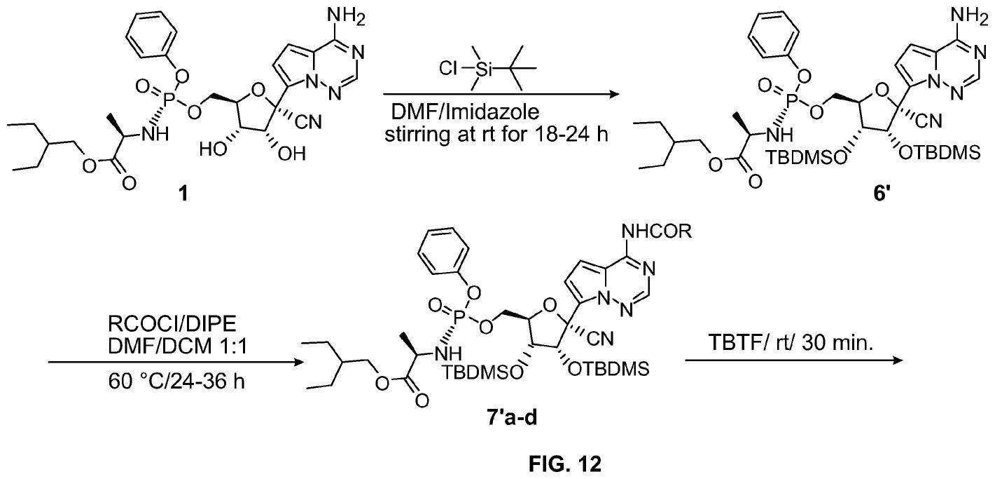

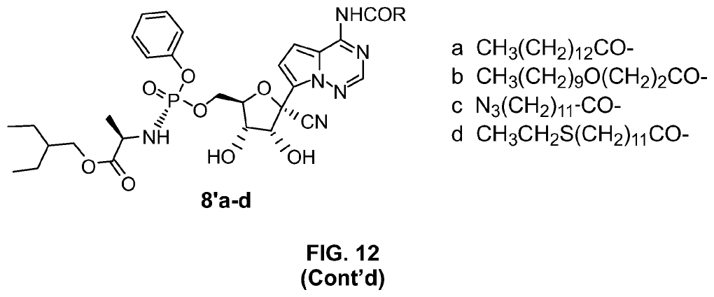

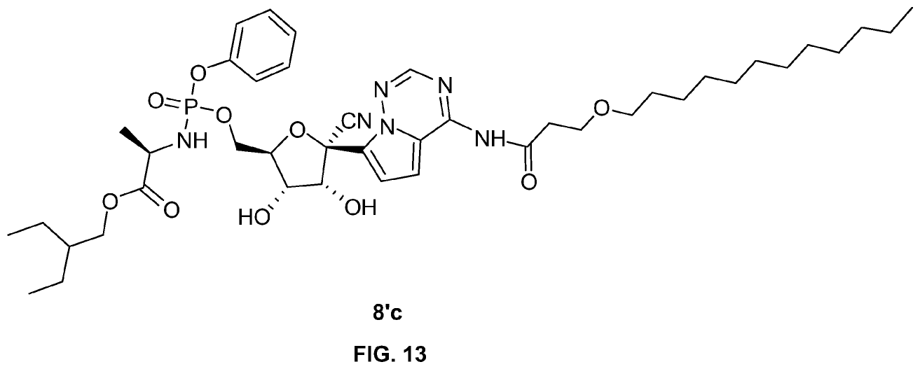

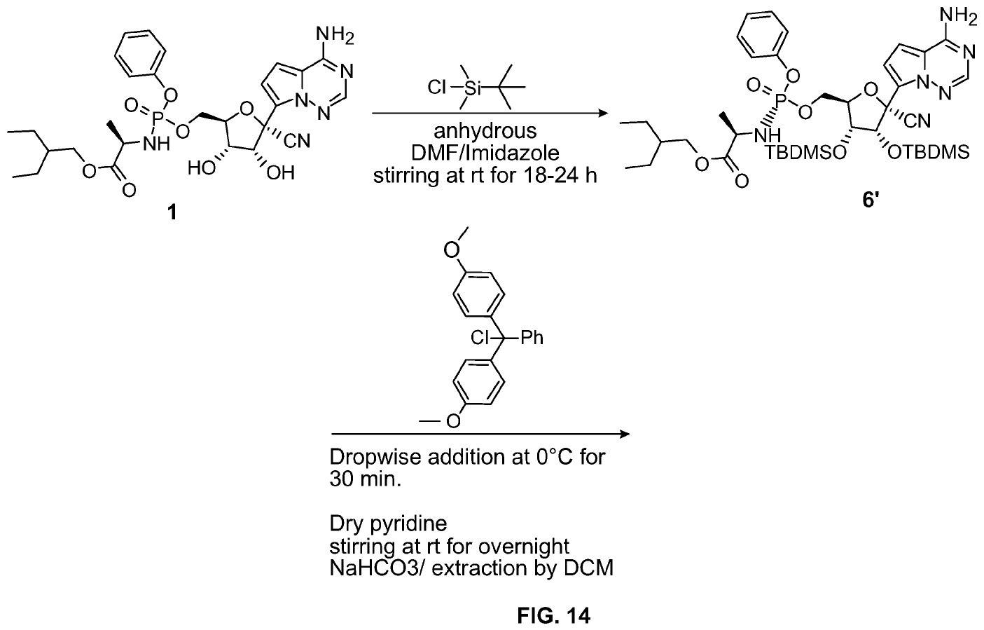

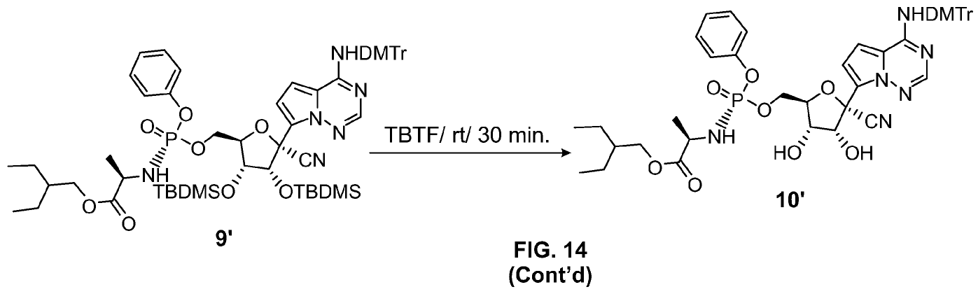

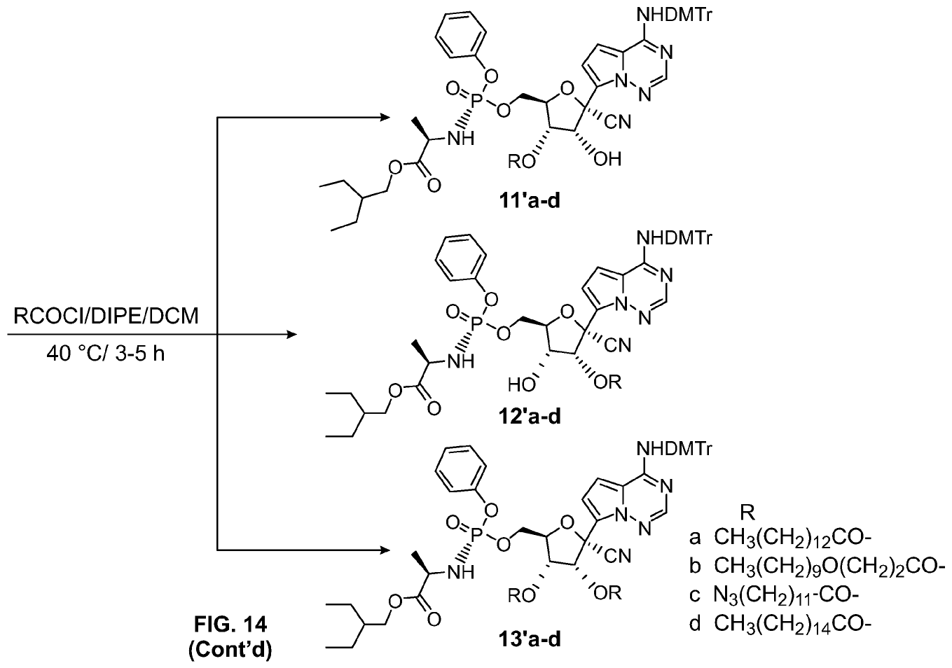

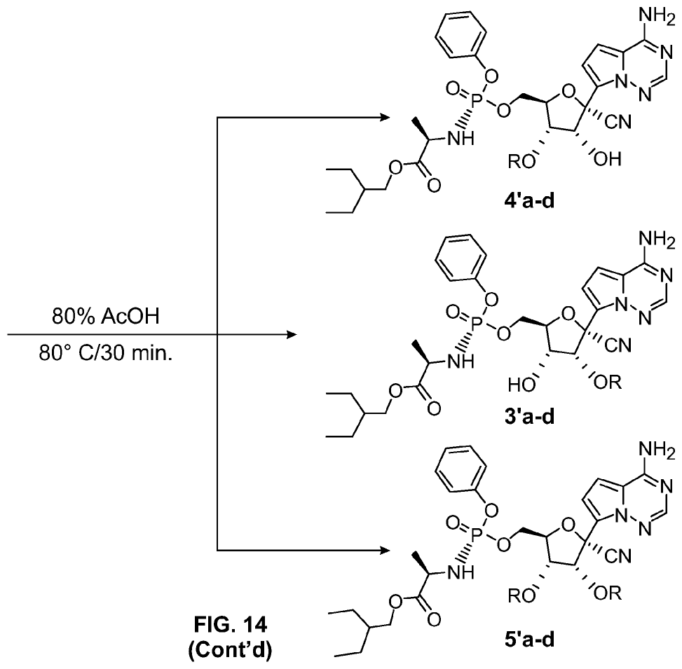

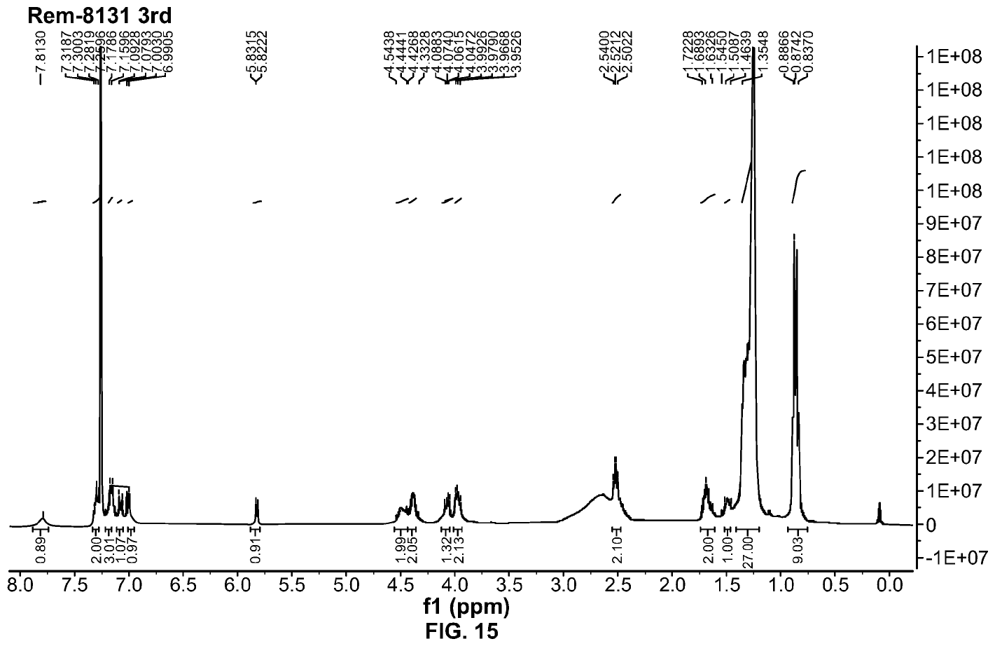

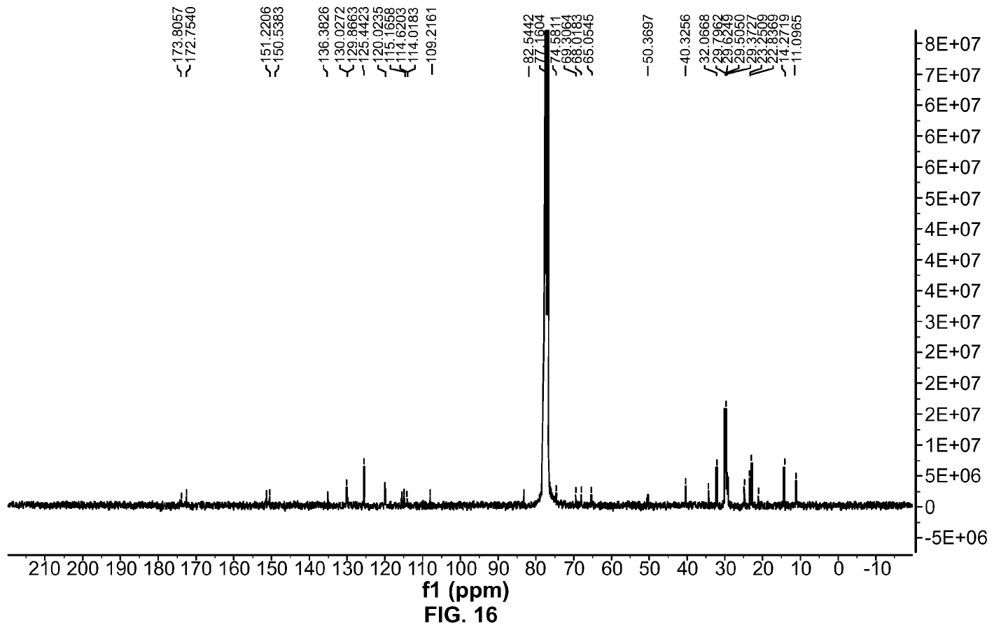

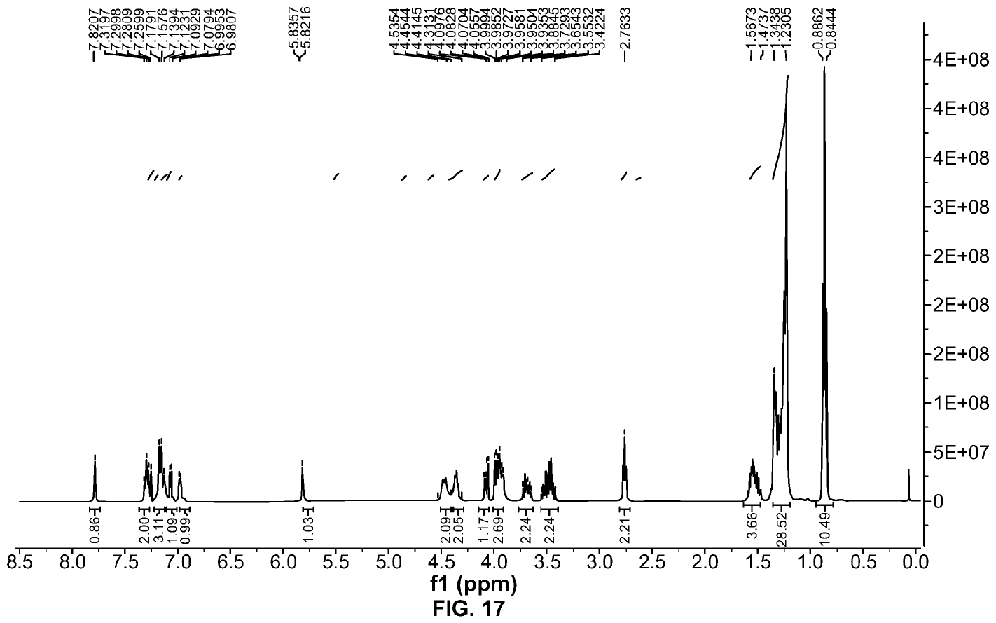

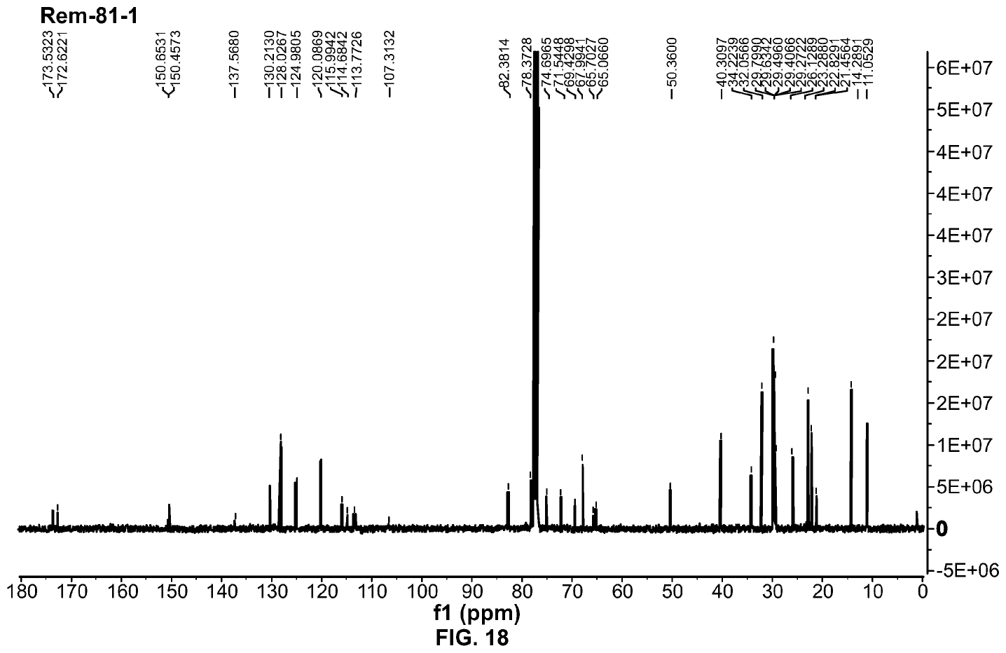

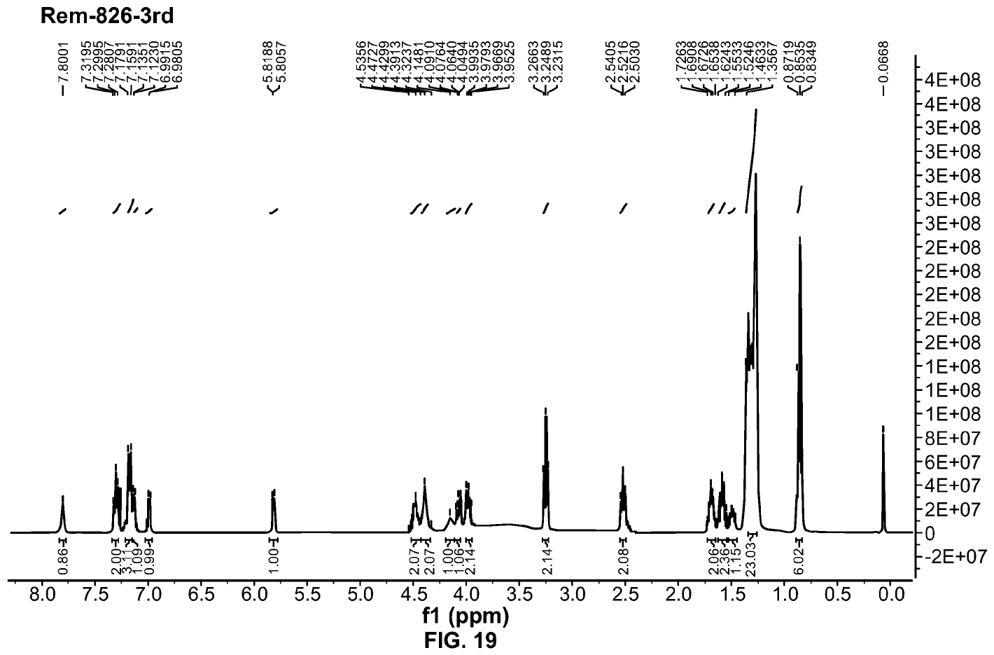

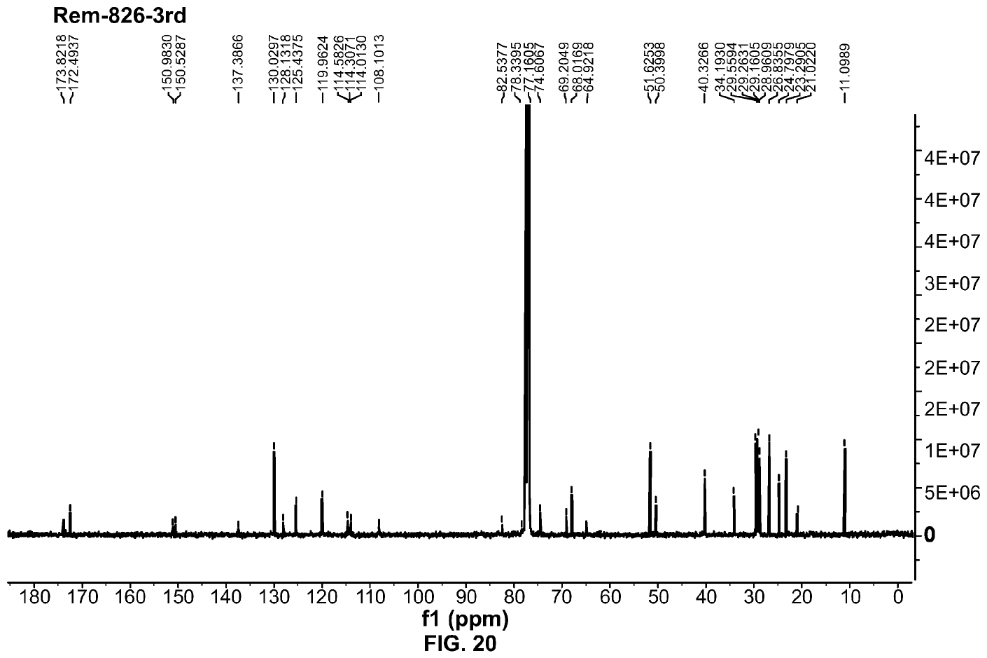

As an example, five different fatty acids; myristic acid, palmitic acid, 4-tetraoxodecanoic acid, 12-azidododecanoic acid, and 12-thioethyldodecanoic acid, in the form of acyl chloride (2′a-e), underwent reaction with the available reactive 2′ and 3′ hydroxyl groups of remdesivir (1). In the second step, fatty acylation was performed by reacting an equimolar amount of the corresponding fatty acyl chloride with a dilute solution of 1 in the presence of diisopropylethylamine (DIPEA) for 5-9 hours to generate mono fatty acyl derivatives that correspond to 2′- and 3′-fatty acyl RDV conjugates as 3′a-e and 4′a-e, respectively. The corresponding compounds from both series have the same molecular weight but differ in their chemical structures. Meanwhile, the use of 3 molar equivalents of fatty acyl chloride and 1 molar equivalent of 1 yielded the 2′,3′-difatty acyl derivatives of RDV (5′a and 5′b) (FIG. 9). Through employing this conjugation plan by controlling the coupling conditions (i.e. reactant concentrations, temperature, and reaction time), we were able to control the target products and the yield. This direct coupling strategy was successful with the five fatty acyl chlorides used (2′a-e) and reduced the number of reaction steps, time, and solvents required for purification. This is in contrast to the protection and deprotection strategy, which involves the selective protection of different functional groups with proper protecting agents, purification of the protected intermediates, coupling with fatty acyl chloride, purification, removal of the protecting group, and purification of the final products in reduced yield.

Mono fatty acyl derivatives 4′a-e were obtained in higher yield as compared to 3′a-e, possibly due to the higher reactivity of the 3′-hydroxyl group than the 2′-hydroxyl group of RDV. The presence of an adjacent strong electron withdrawing group (CN) at C-1′ in 1 reduces the nucleophilicity of the 2′-hydroxyl group; therefore, the carboxy chloride of the fatty acid tends to react more efficiently with the 3′-hydroxy group. The two structural isomers for each fatty acyl monoconjugate were separated on semi-preparative reverse-phase high-performance liquid chromatography (RP-HPLC) and fully characterized using nuclear magnetic resonance (NMR) and mass spectroscopy.

### Materials

Compound 1 (RDV) was purchased from Hangzhou Molcore Biopharmatech Co., Ltd (Hangzhou, China). 12-Bromododecanoic acid, 2-ethanethiol, sodium azide, oxalyl chloride were purchased from MilliporeSigma (USA). All the other reagents, including solvents, were purchased from Fisher Scientific (USA). The final products were purified on a Phenomenex Gemini 10 μm ODS reversed-phase column (2.1×25 cm) with semi-preparative Shimadzu HPLC system using a gradient system at a constant flow rate of 7 mL/min for derivatives 3′a-e and 4′a-e, and 9 mL/min for products 5′a and 5′b.

The purity of the compounds was confirmed by using a Shimadzu analytical HPLC system on a C18 column (Phenomenex Synergi™ 4 μm Hydro-RP 80 Å, LC Column 250×4.6 mm) using a gradient system (water/acetonitrile) at a constant flow rate of 1 mL/min with UV detection at 254 nm. Analytical HPLC confirmed the purity of the final products. The chemical structures of final products were characterized by nuclear magnetic resonance spectrometry 1D NMR (H, ¹³C), and 2D NMR spectra (HSQC, HSQC-TOCSY, HMBC, COSY) measured on a Bruker NMR spectrometer (400 MHz). The chemical shifts were reported in parts per millions (ppm). The compounds' molecular weight was confirmed by a high-resolution mass spectroscopy time-of-flight electrospray mass spectrometer using (Phenomenex Luna, 4 um C18 150×4.6 mm HPLC Column).

### Preparation of Fatty Acyl Chloride

The appropriate fatty acid (3.0 mmol) was dissolved in 5 mL of anhydrous benzene. Then oxalyl chloride (300 μL, 3.6 mmol) was added to the solution of fatty acid, and the reaction mixture was stirred at room temperature (25° C.) for 5 h. The solvents were evaporated to dryness under reduced pressure to get the corresponding fatty acid chloride as yellow syrup.

### General Procedure for the Synthesis of Monofatty Acyl RDV Conjugates 3′a-e and 4′a-e

Remdesivir (1, 61 mg, 0.1 mmol) was dissolved in an anhydrous DCM/THF (2:1, 40 mL) followed by addition of DIPEA (8 equiv, 140 μL, 0.8 mM). The freshly prepared acid chloride (2′a-e, 1.5 equiv, 0.15 mmol) was dissolved in (10 mL) of anhydrous DCM and added to the mixture dropwise over 60 min. The reaction mixture was kept stirring at 40° C. for 6-9 h. LC/MS was used to monitor the progress of the reaction. Upon completing the reaction, the reaction mixture was cooled down to room temperature, the solvents were evaporated to dryness, and the crude product was dissolved in acetonitrile+water+0.1% trifluoroacetic acid (TFA). A C₁₈ reverse phase column was used for purification and separation of different isomers of conjugates 3′a-e and 4′a-e using a gradient of acetonitrile/water containing 0.1% TFA at the flow rate of 7 mL/min using a semipreparative HPLC system.

(2R,3R,4R,5R)-2-(4-aminopyrrolo[2,1-f][1,2,4]triazin-7-yl)-2-cyano-5-((((((R)-1-(2-ethylbutoxy)-1-oxopropan-2-yl)amino)(phenoxy)phosphoryl)oxy)methyl)-4-hydroxytetra-hydrofuran-3-yl tetradecanoate (3′a). Compound 3′a was obtained by the reaction of 1 with myristoyl chloride (2′a). Yield (3.23 mg, 5.3%, colorless solid). ¹H NMR (400 MHz, CDCl₃) δ 7.81 (s, 1H), 7.30 (t, J=7.6 Hz, 2H), 7.17 (d, J=7.6 Hz, 3H), 7.09 (d, J=5.6 Hz, 1H), 6.99 (d, J=4.8 Hz, 1H), 5.82 (d, J=5.6 Hz, 1H), 4.54-4.44 (m, 2H), 4.42-4.35 (m, 2H), 4.06 (dd, J=6.0, 6.0 Hz, 1H), 3.98 (dd, J=5.6, 6.0 Hz, 1H), 3.95-3.90 (m, 1H), 2.62 (br, 1H, NH), 2.52 (t, J=7.6 Hz, 2H), 1.72-1.63 (m, 2H), 1.54-1.46 (m, 1H), 1.36-1.25 (m, 27H), 0.89-0.84 (m, 9H). ¹³C NMR (101 MHz, CDCl₃) δ 173.81, 172.75, 151.22, 150.54, 136.38, 130.03, 129.87, 125.44, 120.02, 115.17, 114.62, 114.02, 109.22, 82.54, 78.36, 74.58, 69.31, 68.02, 65.05, 50.37, 40.33, 34.22, 32.07, 29.80, 29.63, 29.51, 29.37, 29.21, 24.83, 23.29, 22.84, 21.06, 14.27, 11.10, HR-MS (ESI-TOF) (m/z): C₄₁H₆₁N₆O₉P, calcd., 812.9458; found, 813.4431 [M+H]⁺.
((2R,3R,4R,5R)-2-(4-aminopyrrolo[2,1-f][1,2,4]triazin-7-yl)-2-cyano-5-((((((R)-1-(2-ethylbutoxy)-1-oxopropanyl)amino)(phenoxy)phosphoryl)oxy)methyl)-4-hydroxytetra-hydro-furan-3-yl 3-(decyloxy)propanoate (3b). Compound 3′b was obtained by the reaction of 1 with 3-(decyloxy)propanoyl chloride (2%). Yield (3.3 mg, 5.3%, colorless solid). ¹H NMR (400 MHz, CDCl₃) δ 7.85 (s, 1H), 7.28 (t, J=7.6 Hz, 2H), 7.15 (dd, J=8.4, 7.6 Hz, 3H), 7.08 (d, J=5.6 Hz, 1H), 6.99 (d, J=5.6 Hz, 1H), 5.83 (d, J=5.6 Hz, 1 H), 4.53-4.43 (m, 2H), 4.43-4.31 (m, 2H), 4.08 (dd, J=5.6, 6.0 Hz, 1H), 4.00-3.95 (m, 2H), 3.94-3.88 (m, 1H), 3.73-3.65 (m, 2H), 3.55-3.42 (m, 2H), 2.76 (t, J=7.6 Hz, 2H), 1.57-1.47 (m, 3H), 1.34-1.23 (m, 24H), 0.89-0.84 (m, 9H). ¹³C NMR (101 MHz, CDCl3) δ 173.53, 172.62, 150.65, 150.46, 137.57, 130.21, 128.03, 124.98, 120.09, 115.99, 114.68, 113.77, 107.31, 82.38, 78.37, 74.70, 71.54, 69.43, 65.70, 67.99, 65.07, 50.36, 40.31, 34.22, 32.06, 29.80 29.63, 29.50, 29.41, 29.27, 26.13, 23.29, 22.83, 21.16, 14.27, 11.05. HR-MS (ESI-TOF) (m/z): C₄₀H₅₉N₆O₁₀P, 814.9178; found, 815.4182 [M+H]⁺.
(2R,3R,4R,5R)-2-(4-aminopyrrolo[2,1-f][1,2,4]triazin-7-yl)-2-cyano-5-((((((R)-1-(2-ethylbutoxy)-1-oxopropan-2-yl)amino)(phenoxy)phosphoryl)oxy)methyl)-4-hydroxytetra hydrofuran-3-yl palmitate (3′c). Compound 3′c was obtained by the reaction of 1 with 12-azidododecanoyl chloride (2′c). Yield (4.27 mg, 7%, colorless solid). ¹H NMR (400 MHz, CDCl₃) δ 7.80 (s, 1H, H-2 of the pyrrolotriazine ring), 7.29 (t, J=7.6 Hz, 2H, phenyl m-Hs), 7.18 (d, J=8.0 Hz, 3H phenyl o- and p-Hs), 7.14 (d, J=4.8 Hz, 1H), 6.99 (d, J=4.4 Hz, 1H), 5.81 (d, J=5.2 Hz, 1H), 4.53-4.43 (m, 2H), 4.43-4.33 (m, 2H), 4.14 (t, 1H) 4.06 (dd, J=5.8, 5.8 Hz, 1H), 3.99 (dd, J=5.7, 5.6 Hz, 2H), 3.25 (t, J=6.8 Hz, 2H), 2.52 (t, J=7.6 Hz, 2H), 1.72-1.65 (m, 2H), 1.62-1.55 (m, 2H), 1.52-1.46 (m, 1H), 1.36-1.27 (m, 23 Hs), 0.86 (t, J=7.4 Hz, 6H). ¹³C NMR (101 MHz, CDCl₃) δ 173.82, 172.49, 150.98, 150.53, 137.39, 130.03 128.13, 125.44, 119.96, 114.58, 114.31, 114.01, 108.10, 82.54, 78.34, 74.61, 69.20, 68.02, 64.92, 51.63, 50.40, 40.33, 34.19, 29.58, 29.26, 29.16, 28.96, 26.84, 24.80, 23.29, 21.02, 11.10. HR-MS (ESI-TOF) (m/z): C₃₉H₅₆N₉O₉P, calcd, 825.9048; found, 826.4096 [M+H]⁺.
(2R,3R,4R,5R)-2-(4-aminopyrrolo[2,1-f][1,2,4]triazin-7-yl)-2-cyano-5-((((((R)-1-(2-ethylbutoxy)-1-oxopropan-2-yl)amino)(phenoxy)phosphoryl)oxy)methyl)-4-hydroxytetra hydrofuran-3-yl palmitate (3′d). Compound 3′d was obtained by the reaction of 1 with palmitoyl chloride (2′d). Yield (6.0 mg, 10%, colorless solid). ¹H NMR (400 MHz, CDCl₃) δ 7.82 (s, 1H), 7.30 (t, J=7.6 Hz, 2H), 7.18 (dd, J=8.0, 8.0 Hz, 3H), 7.11 (d, J=4.4 Hz, 1H), 6.98 (d, J=4.8 Hz, 1H), 5.82 (d, J=5.6 Hz, 1H), 4.52-4.45 (m, 2H), 4.40-4.30 (m, 2H), 4.07 (dd, J=6.0, 6.0 Hz, 1H), 4.00 (dd, J=5.6, 5.6 Hz, 1H), 3.93-3.86 (m, 1H), 2.50 (t, J=7.6 Hz, 2H), 1.71-1.59 (m, 2H), 1.53-1.47 (m, 1H), 1.37-1.25 (m, 31H), 0.89-0.84 (m, 9H). ¹³C NMR (101 MHz, CDCl3) δ 173.46, 172.52150.80, 150.46, 137.22, 130.05, 128.43, 125.45, 120.05, 115.20, 114.57, 114.11, 10811, 82.50, 78.33, 74.65, 69.30, 68.02, 65.00, 50.42, 40.33, 34.22, 32.07, 29.79, 29.64, 29.36, 29.23, 2917, 29.10, 24.84, 23.26, 22.84, 21.08, 14.27, 11.06. HR-MS (ESI-TOF) (m/z): C₄₃H₆₅N₆O₉P, 840.9866; found, 841.4703 [M+H]⁺.
(2R,3R,4R,5R)-2-(4-aminopyrrolo[2,1-f][1,2,4]triazin-7-yl)-2-cyano-5-(((((®-1-(2-ethyl-butoxy)-1-oxopropanyl)amino)(phenoxy)phosphoryl)oxy)methyl)-4-hydroxy-tetrahydro-furan-3-yl 12-(ethylthio)dodecanoate (3′e). Compound 3′e was obtained by the reaction of 1 with 12-(ethylthio)dodecanoyl chloride (2′e). Yield (5.49 mg, 9%, colorless solid). ¹H NMR (400 MHz, CDCl₃) δ 7.79 (s, 1H), 7.30 (t, J=7.6 Hz, 2H), 7.19 (dd, J=7.2, 8.4 Hz, 3H), 7.11 (d, J=4.8 Hz, 1H), 6.93 (d, J=4.8 Hz, 1H), 5.84 (d, J=5.6 Hz, 1H), 4.51-4.44 (m, 2H), 4.39-4.30 (m, 2H), 4.18 (t, 1H), 4.06 (dd, J=6.0, 6.0 Hz, 1H), 3.97 (dd, J=5.6, 6.0 Hz, 1H), 3.94-3.89 (m, 1H), 2.56-2.45 (m, 6H), 1.72-1.65 (m, 2H), 1.61-1.53 (m, 2H), 1.51-1.46 (m, 1H), 1.36-1.23 (m, 24H), 0.88-0.83 (dt, J=4.36, 7.4 Hz, 6H). ¹³C NMR (101 MHz, CDCl₃): δ 173.49, 172.58, 151.51, 150.50, 138.65, 130.00, 127.30, 125.38, 120.06, 115.44, 114.76, 116.66, 107.36, 82.53, 78.47, 74.58, 69.27, 67.96, 65.72, 50.36, 40.18, 34.11, 31.80, 29.78, 29.64, 29.58, 29.30, 29.22, 29.17, 29.09, 26.05, 24.83, 23.28, 21.10, 14.96, 11.04. HR-MS (ESI-TOF) (m/z): C₄₁H₆₁N₆O₉PS 844.9995; found, 845.4168 [M+H]⁺.
(2R,3S,4R,5R)-5-(4-aminopyrrolo[2,1-f][1,2,4]triazin-7-yl)-5-cyano-2-((((((R)-1-(2-ethylbutoxy)-1-oxopropan-2-yl)amino)(phenoxy)phosphoryl)oxy)methyl)-4-hydroxy tetrahydro-furan-3-yl tetradecanoate (4′a). Compound 4′a was obtained by the reaction of 1 with myristoyl chloride (2′a). Yield (39.6 mg, 65%, colorless solid). ¹H NMR (400 MHz, CDCl₃) δ 7.85 (s, 1H), 7.19 (t, J=7.6 Hz), 7.07 (dd, J=5.6, 3.2 Hz, 3H), 7.01 (d, J=5.2 Hz, 1H), 6.92 (d, J=4.4 Hz, 1H), 5.33 (dd, J=2.8, 2.8 Hz, 1H), 4.71 (d, J=6 Hz, 1H), 4.60-4.54 (br. m, 1H), 4.41-4.30 (m, 2H), 4.07 (dd, J=5.6 Hz, 5.6 Hz, 1H), 3.99-3.94 (m, 2H), 3.93-3.88 (m, 1H), 2.48 (dt, J=7.6 Hz, 2.8 Hz, 2H), 1.73-1.65 (m, 2H), 1.53-1.47 (m, 1H), 1.35-1.25 (m, 27H), 0.89-0.85 (m, 9H). ¹³C NMR (101 MHz, CDCl3) δ 173.64, 173.54, 152.83, 150.46, 141.79, 130.01, 129.81, 125.23, 120.08, 115.58, 114.88, 112.92 105.55, 83.28, 78.67, 75.04, 72.39, 67.93, 65.77, 50.37, 40.31, 34.19, 32.06, 29.80, 29.63, 29.50, 29.40, 29.26, 24.85, 23.25, 22.83, 21.12, 14.27, 11.10. HR-MS (ESI-TOF) (m/z): C₄₁H₆₁N₆O₉P calcd., 812.4635 found, 813.4425 [M+H]⁺.
(2R,3S,4R,5R)-5-(4-aminopyrrolo[2,1-f][1,2,4]triazin-7-yl)-5-cyano-2-((((((R)-1-(2-ethylbutoxy)-1-oxopropan-2-yl)amino)(phenoxy)phosphoryl)oxy)methyl)-4-hydroxy-tetrahydrofuran-3-yl-3-(decyloxy)propanoate (4′b). Compound 4′b was obtained by the reaction of 1 with 3-(decyloxy)propanoyl chloride (2′b). Yield (32.3 mg, 53%, colorless solid). ¹H NMR (400 MHz, CDCl₃) δ 7.86 (s, 1H), 7.24 (t, J=7.6 Hz, 2H), 7.09 (t, J=8 Hz, 3H), 7.04 (d, J=3.2 Hz, 1H), 6.98 (d, J=4.0 Hz, 1H), 5.53 (dd, J=2.0, 2.4 Hz, 1H), 4.84 (d, J=5.6 Hz, 1H), 4.64-4.57 (br. m, 1H), 4.40-4.30 (m, 2H), 4.08 (dd, J=6.0, 6.0 Hz, 1H), 3.99 (dd, J=6.0, 5.6 Hz, 1H), 3.96-3.90 (m, 2H), 3.80-3.66 (m, 2H), 3.55-3.42 (m, 2H), 2.77 (t, J=6.0 Hz, 2H), 1.55-1.46 (m, 3H), 1.35-1.25 (m, 24H), 0.90-0.85 (m, 9H). ¹³C NMR (101 MHz, CDCl₃) δ 173.48, 170.42, 151.72, 150.45, 138.99, 129.91, 127.63, 125.36, 120.15, 115.37, 114.01, 113.86, 107.01, 82.83, 78.27, 75.41, 72.01, 71.72, 65.86, 67.95, 65.69, 50.39, 40.33, 35.43, 32.03, 29.85, 29.72, 29.55, 29.45, 26.06, 23.26, 22.82, 21.20, 14.26, 11.06. HR-MS (ESI-TOF) (m/z): C₄₀H₅₉N₆O₁₀P 814.9178; found, 815.4215 [M+H]⁺.
(2R,3S,4R,5R)-5-(4-aminopyrrolo[2,1-f][1,2,4]triazin-7-yl)-5-cyano-2-((((((R)-1-(2-ethylbutoxy)-1-oxopropan-2-yl)amino)(phenoxy)phosphoryl)oxy)methyl)-4-hydroxy-tetrahydrofuran-3-yl-12-azidododecanoate (4′c). Compound 4′c was obtained by the reaction of 1 with 12-azidododecanoyl chloride (2′c). Yield (30.50 mg, 50%, colorless solid). ¹H NMR (400 MHz, CDCl₃) δ 7.84 (s, 1H), 7.22 (t, J=7.2 Hz, 2H), 7.18 (d, J=7.6 Hz, 1H), 7.06 (dd, J=6.8, 7.6 Hz, 3H), 6.98 (d, J=3.6 Hz, 1H), 5.30 (dt, J=2.8, 3.2 Hz, 1H), 4.75 (d, J=5.2 Hz, 1H), 4.62-4.54 (br. m, J=5.6 Hz, 1H), 4.44-4.29 (m, 2H), 4.06 (dd, J=6.0, 6.0 Hz, 1H), 3.99 (dd, J=5.6, 5.6 Hz, 1H), 3.93-3.85 (m, 1H), 3.81 (d, J=7.6 Hz, 1H), 3.25 (t, J=6.8 Hz, 2H), 2.48 (t, J=7.6 Hz, 2H), 1.71-1.66 (m, 2H), 1.61-1.55 (m, 2H), 1.52-1.46 (m, 1H), 1.35-1.25 (m, 23H), 0.87 (t, J=7.6 Hz, 6H). ¹³C NMR (101 MHz, CDCl₃) δ 173.63, 173.51, 150.83, 150.31, 137.73, 129.94, 128.40, 125.42, 120.06, 115.28, 114.15, 113.90, 108.62, 83.23 78.35, 74.93, 72.23, 67.99, 65.81, 51.62, 50.37, 40.31, 34.16, 29.84, 29.57, 29.33, 29.27, 28.96, 26.84, 24.82, 23.24, 20.99, 11.10. HR-MS (ESI-TOF) (m/z): C₃₉H₅₆N₉O₉P: 825.9048, 826.4135 [M+H]⁺.
(2R,3S,4R,5R)-5-(4-aminopyrrolo[2,1-f][1,2,4]triazin-7-yl)-5-cyano-2-((((((R)-1-(2-ethylbutoxy)-1-oxopropan-2-yl)amino)(phenoxy)phosphoryl)oxy)methyl)-4-hydrox-ytetrahydrofuran-3-yl pentadecanoate (4′d). Compound 4′d was obtained by the reaction of 1 with palmitoyl chloride (2′d). Yield (39.0 mg, 64%, colorless solid). ¹H NMR (400 MHz, CDCl₃) δ 7.86 (s, 1H, H-2 of the pyrrolotriazine ring), 7.22 (t, J=7.6 Hz, 2H), 7.18 (d, J=7.6 Hz, 1H), 7.09 (dd, J=7.2, 8.0 Hz, 3H), 6.98 (d, J=4.8 Hz, 1H), 5.30 (dd, 1H, J=2.4, 2.8 Hz,), 4.76 (d, J=6.0 Hz, 1H), 4.60-4.53 (br. m, 1H), 4.45-4.29 (m, 2H), 4.06 (dd, J=5.6, 6.0 Hz, 1H), 3.99 (dd, J=5.6, 6.0 Hz, 1H), 3.93-3.85 (m, 1H), 2.46 (t, J=7.6 Hz, 2H), 1.72-1.62 (m, 2H), 1.53-1.47 (m, 1H), 1.33-1.25 (m, 31H), 0.89-0.85 (m, 9H). ¹³C NMR (101 MHz, CDCl₃) δ 173.51, 173.43, 150.37, 150.27, 136.44, 129.95, 129.13, 125.52, 120.04, 114.99, 114.22, 113.79, 109.26, 83.29, 78.22, 75.10, 72.13, 68.04, 65.87, 50.35, 40.31, 34.15, 32.07, 29.81, 29.63, 29.51, 29.39, 29.25, 24.84, 23.23, 22.84 (CH2-15 of the fatty acid moiety), 20.89, 14.27, 11.02. HR-MS (ESI-TOF) (m/z): C₄₃H₆₅N₆O₉P, 840.9998; found, 841.4892 [M+H]⁺.
((2R,3S,4R,5R)-5-(4-aminopyrrolo[2,1-f][1,2,4]triazin-7-yl)-5-cyano-2-((((((R)-1-(2-ethyl-butoxy)-1-oxopropan-2-yl)amino)(phenoxy)phosphoryl)oxy)methyl)-4-hydroxy-tetrahydro-furan-3-yl 12-(ethylthio)dodecanoate (4′e). Compound 4′e was obtained by the reaction of 1 with 12-(ethylthio)dodecanoyl chloride (2′e). Yield (33.6 mg, 55%, colorless solid). ¹H NMR (400 MHz, CDCl₃) δ 7.83 (s, 1H), 7.23 (t, J=7.6 Hz, 2H), 7.17 (d, J=7.2 Hz, 1H), 7.08 (dd, J=7.6, 7.2 Hz, 3H), 6.96 (d, J=4.0 Hz, 1H), 5.31 (dd, J=2.8, 2.8 Hz, 1H), 4.81 (d, J=5.2 Hz, 1H), 4.61-4.53 (m, J=6.0 Hz, 1H), 4.41-4.34 (m, 2H), 4.06 (dd, J=5.6, 5.6 Hz, 1H), 3.98 (dd, J=6.0, 5.6 Hz, 1H), 3.94-3.90 (m, 1H), 2.52-2.43 (m, 6H), 1.72-1.63 (m, 2H), 1.61-1.53 (m, 2H), 1.52-1.47 (m, 1H), 1.34-1.23 (m, 24H), 0.87 (t, J=7.6 Hz, 6H). ¹³C NMR (101 MHz, CDCl3), δ 173.85, 173.48, 150.71, 150.38, 137.96, 129.93, 128.42, 125.41, 120.07, 115.23, 114.17, 113.83, 108.41, 83.24, 78.34, 75.07, 72.23, 67.99, 65.82, 50.39, 40.32, 34.17, 33.74, 32.96, 31.80, 29.80, 29.65, 29.57, 29.36, 29.24, 29.11, 28.89, 28.30, 26.04, 24.83, 23.25, 21.09, 14.98, 11.10. HR-MS (ESI-TOF) (m/z): C₄₁H₆₁N₆O₉PS 844.9995; found, 845.4116 [M+H]⁺.
General procedure for the synthesis of di fatty acyl remdesivir conjugates 5′a-b. A solution of 1 (20.40 mg, 0.033 mmol) was dissolved in an anhydrous DCM/THF (2:1, 20 mL) containing DIPEA (˜8 equiv, 47 μL, 0.27 mM). Then this solution o was added gradually on three portions to a solution of freshly prepared fatty acyl chloride (5 equiv, 0.165 mmol) dissolved in (10 mL) of anhydrous DCM. The reaction mixture was kept stirring at 40° C. for 6-9 h. LC/MS was used to monitor the progress of the reaction. Upon completing the reaction, the reaction mixture was cooled down to room temperature, the solvents were evaporated to dryness, and the crude product was dissolved in acetonitrile+water+0.1% TFA. The crude compound was purified using a C18 column as mentioned above.
((2R,3R,4R,5R)-2-(4-aminopyrrolo[2,1-f][1,2,4]triazin-7-yl)-2-cyano-5-((((((R)-1-(2-ethylbutoxy)-1-oxopropan-2-yl)amino)(phenoxy)phosphoryl)oxy)methyl)tetrahydro-furan-3,4-diyl ditetradecanoate (5′a). Compound 5′a was obtained by the reaction of 1 with myristoyl chloride (2′a). Yield (˜17.4 mg, 85.0%, colorless solid). ¹H NMR (400 MHz, CDCl₃) δ 7.94 (s, 1H), 7.29 (t, J=7.6, 2H), 7.15 (dd, J=8.4, 7.2 Hz, 3H), 6.85 (d, J=4.4, 1H), 6.57 (d, J=4.8 Hz, 1H), 6.22 (d, J=5.6 Hz, 1H), 6.15-5.90 (br s, 2H), 5.52 (dt, J=4.8, 0.8 Hz), 4.58 (dd, J=4.0, 4.0 Hz 1H), 4.39 (dd, J=5.6, 4.4 Hz, 2H), 4.06 (dd, J=5.6, 6.0 Hz, 1H), 4.01 (d, J=3.2 Hz, 1H), 3.99 (dd, J=6.0, 6.0 Hz, 2H), 2.42-2.35 (m, 4H), 1.52-1.45 (m, 1H), 1.36-1.24 (m, 47H), 0.89-0.84 (m, 12 Hs). ¹³C NMR (101 MHz, CDCl₃) δ 173.58, 172.71, 171.87, 150.69, 150.62, 146.95, 129.85, 125.12, 122.11, 120.23, 117.02, 115.32, 112.78, 100.77, 82.07, 71.99, 70.45, 67.75, 65.45, 50.36, 40.31, 34.01, 32.06, 29.80, 29.77, 29.63, 29.50, 29.45, 29.39, 29.20, 24.86, 24.75 23.29, 22.72, 21.25, 14.26, 11.06. HR-MS (ESI-TOF) (m/z): C₅₅H₈₇N₆O₁₀P, calcd., 1023.3068; found, 1023.6637 [M+H]⁺.
(2R,3R,4R,5R)-2-(4-aminopyrrolo[2,1-f][1,2,4]triazin-7-yl)-2-cyano-5-((((((R)-1-(2-ethylbutoxy)-1-oxopropan-2-yl)amino)(phenoxy)phosphoryl)oxy)methyl)tetrahydrofuran-3,4-diyl ditetradecanoate (5%). Compound 5′b was obtained by the reaction of 1 with 12-azidododecanoyl chloride (2′c). Yield (15.3 mg, 75%, colorless solid). ¹H NMR (400 MHz, CDCl3) δ 7.76 (s, 1H), 7.28 (t, J=8.0 Hz, 2H), 7.14 (t, J=2.8, 5.6 Hz, 3H), 7.07 (d, J=4.8 Hz, 1H), 6.90 (d, J=4.8 Hz, 1H), 6.01 (d, J=6.4 Hz, 1H), 5.46 (dt, J=4.4, 1.6 Hz, 1H), 4.65-4.56 (m, 1H), 4.42-4.38 (m, 2H), 4.09 (dd, J=6.0, 6.0 Hz, 1H), 4.05-3.95 (m, 2H), 3.81 (d, J=3.2 Hz, 1H), 3.25 (dt, J=6.8, 2.4 Hz, 4H), 2.42-2.35 (m, 4H), 1.69-1.55 (m, 8H), 1.53-1.47 (m, 1H), 1.39-1.25 (m, 39H), 0.88 (t, J=7.6 Hz, 6H). ¹³C NMR (101 MHz, CDCl₃) δ 173.47, 172.71, 171.73, 150.85, 150.37, 137.26, 130.01, 126.89, 125.44, 120.19, 114.63 114.51, 114.29, 107.99, 82.66, 78.91, 72.62, 70.06, 67.92, 65.41, 51.61, 50.42, 40.33, 34.03, 33.92, 32.06, 29.79, 29.60, 29.40, 29.28, 29.18, 28.97, 28.79, 26.85 & 24.84, 24.70, 23.30, 22.84, 11.10. HR-MS (ESI-TOF) (m/z): C₅₁H₇₇N₁₂O₁₀P, 1049.2077; found, 1049.6035 [M+H]⁺.

The attachment of the long-chain fatty acid analogs to RDV enhanced their lipophilicity as shown by calculated partition coefficients (C Log P) (Table 1). Difatty acyl conjugates (5′a and 5′b) exhibited significantly higher lipophilicity due to a large number of methylene chains as compared with a smaller number of methylene chains in mono fatty acyl conjugates 3′a-e and 4′a-e (Table 1). Monopalmitoyl conjugates Ye and 4′e were more lipophilic than mono fatty acyl conjugates with shorter chain lengths, such as tetradecanoyl derivatives 3′a and 4′a. The higher lipophilicity is expected to improve the permeability properties in order to achieve a higher concentration of the prodrug in the infected cells. The enhanced lipophilicity of these conjugates, relative to RDV, should increase their ability to cross the plasma membrane. This postulate is based on the observation that increasing the lipophilicity of FTC, FLT, and 3TC improved cell permeability.

TABLE 1
Calculated partition coefficient of fatty acyl Remdesivir conjugates.

| | | CLogP |
|---|---|---|
| Compound | Fatty acyl chain | (calcd)^{a} |
| RDV | H | 1.25 |
| 3′a | CH₃(CH₂)₁₂CO— | 8.45 |
| 3′b | CH₃(CH₂)₉O(CH₂)₂CO— | 6.83 |
| 3′c | N₃(CH₂)₁₁CO— | 7.66 |
| 3′d | CH₃(CH₂)₁₄CO— | 9.51 |
| 3′e | CH₃CH₂S(CH₂)₁₁CO— | 7.54 |
| 4′a | CH₃(CH₂)₁₂CO— | 8.45 |
| 4′b | CH₃(CH₂)₉O(CH₂)₂CO— | 6.83 |
| 4′c | N₃(CH₂)₁₁CO— | 7.67 |
| 4′d | CH₃(CH₂)₁₄CO— | 9.51 |
| 4′e | CH₃CH₂S(CH₂)₁₁CO— | 7.54 |
| 5′a | CH₃(CH₂)₁₂CO— | 15.65 |
| 5′b | N₃(CH₂)₁₁CO— | 14.08 |

^{a}Calculated partition coefficient using ChemBioDraw Ultra 20.0.

### Additional Embodiments

In view of the described compositions and methods, hereinbelow are described certain more particularly described embodiments of the inventions. These particularly described embodiments should not, however, be interpreted to have any limiting effect on any different claims containing different or more general teachings described herein, or that the “particular” embodiments are somehow limited in some way other than the inherent meanings of the language and formulas literally used therein.

Embodiment 1. A compound comprising at least one substituted nucleoside selected from the group consisting of Formulas I or II

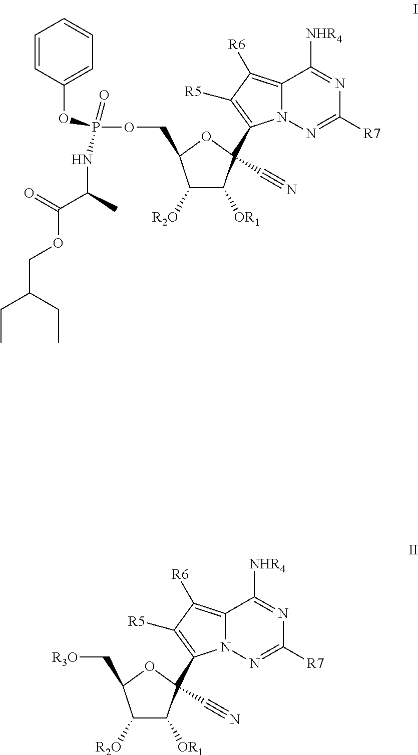

wherein

R₁, R₂, and R₃ are each independently selected from Z-CO-, an anionic polymer complexed with a cleavable linker, a fatty acid analog complexed with a cleavable linker, a fatty alcohol analog complexed with a cleavable linker, a carboxylic ester side chain of a linear or cyclic peptide, a polycarboxylic ester aryl or heteroaryl, carbopol, or a phosphodiester; wherein

the fatty acid analog is selected from the group consisting of X′(CH₂)ₙY′(CH₂)nCO- and CH₃(CH₂)ₙCH(Br)CO—;

the fatty alcohol analog is selected from the group consisting of X′_{(CH2)}nY′(CH₂)ₙCH₂O- and CH₃(CH₂)ₙCH(Br)CH₂O—;

Z is selected from X(CH₂)ₙY(CH₂)ₙ—, CH₃(CH₂)ₙCH(Br)—, suramin, cellulose acetate, or an anionic polymer;

X is selected from CH₃, N₃, alkyl-S, alkyl-O, aryl-O, aryl-S, alkyl-NH, aryl-NH, Br, Cl, F, I, OH, NH₂, COOH, CHO, CH₃S, aryl, heteroaryl, phenyl, substituted phenyl, alkyne, alkene, suramin, cellulose acetate, or an anionic polymer;

X′ is selected from CH₃, N₃, alkyl-S, alkyl-O, aryl-O, aryl-S, alkyl-NH, aryl-NH, Br, Cl, F, I, OH, NH₂, COOH, CHO, CH₃S, aryl, heteroaryl, phenyl, substituted phenyl, suramin, cellulose acetate, or an anionic polymer;

Y is selected from CH₂, O, S, NH, 1,2,3-triazole;

Y′ is selected from CH₂, O, S, NH;

independently, n is selected from 0-18;

R₄ is selected from H or R₁; and

R₅, R₆, and R₇ are each independently selected from H, N₃, F, CN, Cl, Br, F, I, OH, NH₂, SH alkyl, alkene, alkyne, aryl, O-alkyl, O-aryl.

Embodiment 2. The compound of embodiment 1, wherein the nucleoside is selected from the group consisting of remdesivir or GS 441524.
Embodiment 3. The compound of embodiment 1 or 2, wherein the substituted nucleoside exhibits to treat an infection or reduce transmission of viral diseases such as coronaviruses or display antiviral activity.
Embodiment 4. The compound of one of embodiments 1 to 3 comprising a fatty acid, fatty alcohol substituent-generating ester, or fatty alcohol substituent-generating ether conjugate.
Embodiment 5. The compound of one of embodiments 1 to 4, wherein one or more fatty acid or fatty alcohol is directly or indirectly coupled to the compound of Formula I or II via attachment to one or more free hydroxyl or amino group.
Embodiment 6. The compound of one of embodiments 1 to 5, wherein the compound comprises a first substitution at a nucleoside base and a second substitution at one of positions 1′, 2′, 3′, 4′, and 5′ of a carbohydrate moiety.
Embodiment 7. The compound of one of embodiments 1 to 6, comprising a double bond between C3′ and C4′ in a carbohydrate moiety.
Embodiment 8. The compound of one of embodiments 1 to 7, wherein the fatty acid of Formula I or II comprises X(CH₂)ₙY(CH₂)ₙCOOH or CH₃(CH₂)ₙCH(Br)COOH and wherein the fatty alcohol is X(CH₂)ₙY(CH₂)ₙCH₂OH or CH₃(CH₂)ₙCH(Br)CH₂OH, wherein n=0 to 21; X=CH₃, N₃, alkyl-S, alkyl-O, aryl-O, aryl-S, alkyl-NH, aryl-NH, Br, Cl, F, I, OH, NH₂, COOH, CHO, CH₃S, aryl, heteroaryl, phenyl, alkene, alkyne, or substituted phenyl; and Y=CH₂, O, S, NH, or 1,2,3-triazole.
Embodiment 9. The compounds of one of embodiments 1 to 8, comprising one to three substituent(s) selected from the group consisting of 2-hydroxybenzyl alcohol, 4,4′-dihydroxy-3,3′-di-(hydroxymethyl)diphenylmethane, 4,6-dihydroxy-1,3-benzenedimethanol, 4,4′,4″-methanetriyltris(2-(hydroxymethyl)phenol), a polycarboxylic acid, tribenzenetriacetic acid, hydroxybenzendicarboxylic acid, [(hydroxyphenylene)dixoy]diacetic acid, tris(carboxymethoxy)benzene, a triazine-tricarboxylic acid derivative, triazinetriyltroxy)triacetic acid, 1,3,5-triazine-2,4,6-tricarboxylic acid), an anionic polymer, cellulose sulfate, cellulose sulfate acetate, dextran sulfate, naphthalene sulfonate derivatives, polystyrene sulfonate, carrageenan, a polycarboxylic acid, polyvinylpyrrolidone, a polyanionic compounds, a polyphosphorylated polymer, suramin, cyclodextrin sulfate, a multisulfated peptide, a multiphosphorylated peptide, and an alkyl chains.
Embodiment 10. The compound of one of embodiments 1 to 9, wherein the compound is acylated on one or more NH₂ and OH group by a fatty acid.
Embodiment 11. The compound of embodiment 11, wherein the fatty acid is selected from the group consisting of 12-azidododecanoic acid, 12-thioethydodecanoic acid, tetradecanoic acid, 4-oxatetradecanoic acid, a fatty acyl chloride, and a fatty acid anhydride, wherein the fatty acid is coupled to the compound using HBTU, DIC, and DIPEA as coupling or activating reagents.
Embodiment 12. The compound of one of embodiments 1 to 11, comprising a mono fatty acyl or fatty ether conjugated at a 3′-, 4′-, or 5′-hydroxyl group.
Embodiment 13. The compound of one of embodiments 1 to 12 comprising a difatty or trifatty acyl or ether conjugated at a 3′-, 4′-, and 5′-hydroxyl group.
Embodiment 14. The compound of one of embodiments 1 to 13 comprising a fatty acyl amide or alkyl amino-substituted conjugate.
Embodiment 15. The compound of one of embodiments 1 to 14, wherein the compound comprises a combination of multiple fatty acyl, fatty ether, and fatty acyl amides each coupled to a hydroxyl or amine group.
Embodiment 16. The compound of one of embodiments 1 to 14, wherein the compound comprises a direct ester or ether derivative of a fatty acid or fatty alcohol, which are coupled to the nucleosides through a linker or scaffolds.
Embodiment 17. The compound of embodiment 16, wherein the linker or scaffold is selected from the group consisting of a phosphoramidate, a phosphotriester, a phosphodiester, a phosphomonoester, a triglyceride, a linear peptide backbone, a cyclic peptide backbone, a hydroxyphenyldicarboxylic acid derivative, a compound comprising one or more multicyclosaligenyl groups, tribenzenetriacetic acid, hydroxybenzendicarboxylic acid, [(hydroxyphenylene)dixoy]diacetic acid, tris(carboxymethoxy)benzene, (triazinetriyltroxy)triacetic acid, triazine-tricarboxylic acid derivatives, such as (triazinetriyltroxy)triacetic acid, and 1,3,5-triazine-2,4,6-tricarboxylic acid.
Embodiment 18. The compound of one of embodiments 1 to 14, wherein the compound is directly or indirectly coupled to a second compound.
Embodiment 19. The compound of embodiment 18, wherein the second compound is selected from the group consisting of a nucleoside, a polymer, a polyanionic molecule, cellulose sulfate, cellulose sulfate acetate, dextran sulfate, a naphthalene sulfonate derivative, polystyrene sulfonate, carrageenan, a polycarboxylic acid, polyvinylpyrrolidone, cyclodextrin sulfate, a polyanionic compound, a polyphosphorylated polymer, suramin, a multisulfated peptide, a multiphosphorylated peptide, and an alkyl chain.
Embodiment 20. The compound of one of embodiments 1 to 19, wherein the compound is coupled to a scaffold selected from the group consisting of a linear or cyclic peptide, and wherein the linear or cyclic peptide comprises glutamic acid or aspartic acid coupled to the nucleoside and serine or threonine coupled to the fatty acid.
Embodiment 21. The compound of embodiment 20, wherein the linear or cyclic peptide comprises up to 25 amino acids.
Embodiment 22. The compound of embodiment 20 or 21, wherein the linear peptide or cyclic peptide comprises L amino acids, D amino acids, or a mixture thereof.
Embodiment 23. The compound of one of embodiments 1 to 22, comprising a scaffold comprising one to three 2-hydroxybenzyl alcohol conjugated through a phosphotriester to the nucleosides or fatty alcohol.
Embodiment 24. The compound of embodiments 23, wherein the phosphotriester is a cyclosaligenyl phosphotriester.
Embodiment 25. The compound of one of embodiments 1 to 24 comprising one or more nucleoside or nucleotide analog directly or indirectly coupled to a scaffold in the absence of a fatty acid or fatty alcohol.
Embodiment 26. A pharmaceutical composition comprising a compound of one of embodiments 1 to 25, in an amount that is effective against a coronavirus.
Embodiment 27. The pharmaceutical composition of embodiment 26, wherein the coronavirus is selected from the group consisting of SARS, SARS-COV2, and MERS.
Embodiment 28. The pharmaceutical composition of embodiment 26 or 27 comprising a carrier or excipient.
Embodiment 29. The pharmaceutical composition of one of embodiments 26 to 28, wherein the pharmaceutical composition is formulated as an injectable, a solid or semi-solid form, a tablet, a film, a gel, a cream, an ointment, a spray, a solution, a suspension, a micellar suspension, a powder or granule, an encapsulated granule, an atomized mist, or a pessary.
Embodiment 30. A method for the synthesis of a compounds of one of embodiments 1 to 24, comprising coupling an analog of a fatty acid or a fatty ether to a nucleoside analog.
Embodiment 31. The method of embodiment 30, wherein the nucleoside analog is remdesivir or GS 441524.
Embodiment 32. The method of embodiment 30 or 31, wherein the fatty acid is coupled to the nucleoside using HBTU, DIC, and DIPEA as a coupling or activating reagent.
Embodiment 33. A method of treating a viral disease, comprising:
obtaining a pharmaceutical formulation comprising a compound of one of embodiments 1 to 25; and
applying an effective amount of the pharmaceutical formulation to an individual in need of treatment.
Embodiment 34. The method of embodiment 33, wherein application comprises topical application of the pharmaceutical formulation to a body surface.
Embodiment 35. The method of embodiment 34, wherein the body surface is a mucus membrane,
Embodiment 36. The method of embodiment 33, wherein application comprises injection of the pharmaceutical formulation.
Embodiment 37. The method of embodiment 36, wherein injection is selected from the group consisting of subcutaneous injection, intramuscular injection, intraocular injection, intravenous injection, and infusion.
Embodiment 38. The method of embodiment 33, wherein application comprises inhalation of the pharmaceutical formulation.
Embodiment 39. The method of one of embodiments 33 to 38, wherein treatment is prophylactic.
Embodiment 40. The method of one of embodiments 33 to 38, wherein the individual has an active viral infection but is asymptomatic.
Embodiment 41. The method of one of embodiments 33 to 40, wherein the pharmaceutical formulation comprises a second active compound.
Embodiment 42. The method of embodiment 41, wherein the second active compound is selected from the group consisting of an antiviral compound, an antibacterial compound, an antifungal compound, an anti-inflammatory compound, and a bronchodilator.
Embodiment 43. The method of embodiment 42, wherein the antiviral compound is a second compound as in one of claims 1 to 25.
Embodiment 44. The compound of one of the embodiments of 1 to 24 that is used as long-acting antiviral agents alone or in combination with other antiviral agents.
Embodiment 45. The compound of one of the embodiments of 1 to 24 that the compound leads to the sustained releases of active substrates at adequate concentrations and higher uptake into infected cells.

### REFERENCES

1. de Wit, E.; Feldmann, F.; Cronin, J.; Jordan, R.; Okumura, A.; Thomas, T., Scott, D.; Cihlar, T.; Feldmann, H. Prophylactic and therapeutic remdesivir (GS-5734) treatment in the rhesus macaque model of MERS-CoV infection. Proc. Natl. Acad. Sci. U.S.A. 2020, 117, 6771-6776.
2. Sheahan, T. P.; Sims, A. C.; Graham, R. L.; Menachery, V. D.; Gralinski, L. E.; Case, J. B.; Leist, S. R.; Pyrc, K.; Feng, J. Y.; Trantcheva, I.; Bannister, R.; Park, Y.; Babusis, D.; Clarke, M. O.; Mackman, R. L.; Spahn, J. E.; Palmiotti, C. A.; Siegel, D.; Ray, A. S.; Cihlar, T.; Jordan, R.; Denison, M. R.; Baric, R. S. Broad-spectrum antiviral GS-5734 inhibits both epidemic and zoonotic coronaviruses. Science Translational Medicine 2017, 9 (396), eaa13653.
3. Grein, J.; Ohmagari, N.; Shin, D.; Diaz, G.; Asperges, E.; Castagna, A., et al. Compassionate use of remdesivir for patients with severe Covid-19. The New England Journal of Medicine. Apr. 10, 2020, doi: 10.1056/NEJMoa2007016.
4. Agostini, M. L.; Andres, E. L.; Sims, A. C.; Graham, R. L.; Sheahan, T. P.; Lu, X., Smith, E. C.; Case, J. B.; Feng, J. Y.; Jordan, R.; Ray, A. S.; Cihlar, T.; Siegel, D.; Mackman, R. L.; Clarke, M. O.; Baric, R. S.; Denison, M. R. Coronavirus susceptibility to the antiviral remdesivir (GS-5734) is mediated by the viral polymerase and the proofreading exoribonuclease. mBio. March 2018, 9(2). pii: e00221-18.
5. Herdewijn, P.; Balzarini, J.; De Clercq, E.; Pauwels, R.; Baba, M.; Broder, S.; Vanderhaeghe, H. 3′-Substituted 2′,3′-dideoxynucleoside analogs as potential anti-HIV (HTLV-III/LAV) agents. J. Med. Chem. 1987, 30, 1270-1278.
6. Parang, K.; Knaus, E. E.; Wiebe, L. I. Synthesis, in vitro anti-HIV activity, and biological stability of 5′-O-myristoyl analog derivatives of 3′-fluoro-2′,3′-dideoxythymidine (FLT) as potential prodrugs of FLT. Nucleosides & Nucleotides 1998, 17, 987-1008.
7. Parang, K.; Wiebe, L. I.; Knaus, E. E.; Huang, J. S.; Tyrrell, D. L. In vitro anti-hepatitis B virus activities of 5′-O-myristoyl analog derivatives of 3′-fluoro-2′,3′-dideoxythymidine (FLT) and 3′-azido-2′,3′-dideoxythymidine (AZT). J. Pharm. Pharmaceut. Sci. 1998, 1, 107-113.
8. Agarwal, H. K.; Doncel, G.; Parang, K. Synthesis and anti-HIV activities of phosphate triester derivatives of 3′-fluoro-2′,3′-dideoxythymidine and 3′-azido-2′,3′-dideoxythymidine. Tetrahedron Lett. 2008, 49, 4905-4907.
9. Agarwal, H. K.; Loethan, K.; Mandal, D.; Doncel, G. F.; Parang, K. Synthesis and anti-HIV activities of fatty acyl ester derivatives of 2′,3′-didehydro-2′,3′-dideoxythymidine. Bioorg. Med. Chem. Lett. 2011, 21, 1917-1921.
10. Agarwal, H. K.; Chhikara, B. S.; Hanley, M. J.; Ye, G.; Doncel, G. F.; Parang, K. Synthesis and biological evaluation of fatty acyl ester derivatives of (−)-2′,3′-dideoxy-3′-thiacytidine. J. Med. Chem. 2012, 55, 4861-4871.
11. Agarwal, H. K.; Chhikara, B. S.; Bhavaraju, S.; Mandal, D.; Doncel, G. F.; Parang, K. Emtricitabine prodrugs with improved anti-HIV activity and cellular uptake. Mol. Pharm. 2013, 10, 467-476.

The compositions and methods of the appended claims are not limited in scope by the specific compositions and methods described herein, which are intended as illustrations of a few aspects of the claims and any compositions and methods that are functionally equivalent are intended to fall within the scope of the claims. Various modifications of the compositions and methods in addition to those shown and described herein are intended to fall within the scope of the appended claims. Further, while only certain representative compositions and method steps disclosed herein are specifically described, other combinations of the compositions and method steps also are intended to fall within the scope of the appended claims, even if not specifically recited. Thus, a combination of steps, elements, components, or constituents may be explicitly mentioned herein; however, other combinations of steps, elements, components, and constituents are included, even though not explicitly stated. The term “comprising” and variations thereof as used herein is used synonymously with the term “including” and variations thereof and are open, non-limiting terms. Although the terms “comprising” and “including” have been used herein to describe various embodiments, the terms “consisting essentially of” and “consisting of” can be used in place of “comprising” and “including” to provide for more specific embodiments of the invention and are also disclosed. Other than in the examples, or where otherwise noted, all numbers expressing quantities of ingredients, reaction conditions, and so forth used in the specification and claims are to be understood at the very least, and not as an attempt to limit the application of the doctrine of equivalents to the scope of the claims, to be construed in light of the number of significant digits and ordinary rounding approaches.

1. A compound of Formula I-a 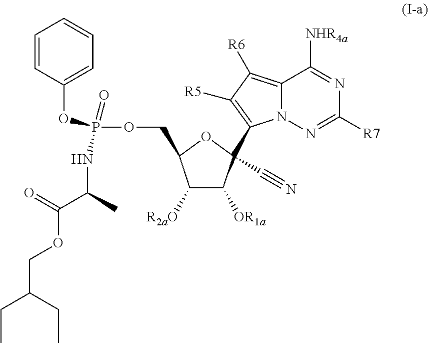
or a pharmaceutically acceptable salt thereof,
wherein:
R₁ₐ, R₂ₐ, and R₄ₐ are each independently selected from hydrogen, Zₐ—, and Zₐ—C(O)—, wherein at least one of Ria, R₂ₐ, and R₄ₐ is not hydrogen;
Zₐ is selected from Xₐ(CH₂)ₙYₐ(CH₂)ₙ—, Xₐ(CH₂)ₙYₐ(CH₂)ₙCH₂—, CH₃(CH₂)ₙCH(Br)—, CH₃(CH₂)ₙCH(Br)CH₂—, suramin, cellulose acetate, or an anionic polymer;
Xₐ is selected from CH₃, N₃, (C₁-C₆ alkyl)-S-, (aryl)-S—, (C₁-C₆ alkyl)-O—, (aryl)-O—, (C₁-C₆ alkyl)-NH—, (aryl)-NH—, Br, Cl, F, I, OH, NH₂, COOH, CHO, aryl, heteroaryl, C₂-C₆ alkynyl, C₂-C₆ alkenyl, suramin, cellulose acetate, or an anionic polymer;
Yₐ is selected from CH₂, O, S, NH, and heteroaryl;
n is independently selected at each occurrence from 0-18; and
R₅, R₆, and R₇ are independently selected from hydrogen, N₃, OH, CN, F, Cl, Br, I, NH₂, SH, C₁-C₆ alkyl, C₂-C₆ alkenyl, C₂-C₆ alkynyl, aryl, —O—(C₁-C₆ alkyl), and —O-(aryl).

2. The compound of claim 1, wherein R₁ₐ is Zₐ— or Zₐ—C(O)—.

3. (canceled)

4. The compound of claim 1, wherein R₂ₐ is Zₐ— or Zₐ—C(O)—.

5. (canceled)

6. The compound of claim 1, wherein R₄ₐ is Zₐ— or Zₐ—C(O)—.

7. (canceled)

8. The compound of claim 1, wherein R₁ₐ is selected from Zₐ— and Zₐ—C(O)—, and R₂ₐ and R₄ₐ are each hydrogen.

9. The compound of claim 1, wherein R₂ₐ is selected from Zₐ— and Zₐ—C(O)—, and R₁ₐ and R₄ₐ are both hydrogen.

10. The compound of claim 1, wherein R₁ₐ and R₂ₐ are each independently selected from Zₐ— and Zₐ—C(O)—, and R₄ₐ is hydrogen.

11. The compound of claim 1, wherein R₄ₐ is selected from Zₐ— and Zₐ—C(O)—, and R₁ₐ and R₂ₐ are both hydrogen.

12. The compound of claim 1, wherein Zₐ is selected from Xₐ(CH₂)ₙYₐ(CH₂)ₙ— and Xₐ(CH₂)ₙYₐ(CH₂)ₙCH₂—.

13. The compound of claim 1, wherein Xₐ is CH₃, N₃, or CH₃CH₂S.

14. (canceled)

15. (canceled)

16. The compound of claim 1, wherein Yₐ is CH₂ or O.

17. (canceled)

18. The compound of claim 1, wherein Zₐ is selected from CH₃(CH₂)₁₁—, CH₃(CH₂)₁₂—, CH₃(CH₂)₁₃—, CH₃(CH₂)₁₄—, CH₃(CH₂)₁₆—, N₃(CH₂)₁₁—, CH₃(CH₂)₉O(CH₂)₂—, CH₃(CH₂)₁₁O(CH₂)₂—, and CH₃CH₂S(CH₂)₁₁—.

19. The compound of claim 1, wherein R₅ is hydrogen, R₆ is hydrogen, or R₇ is hydrogen.

20. (canceled)

21. (canceled)

22. The compound of claim 1 selected from: 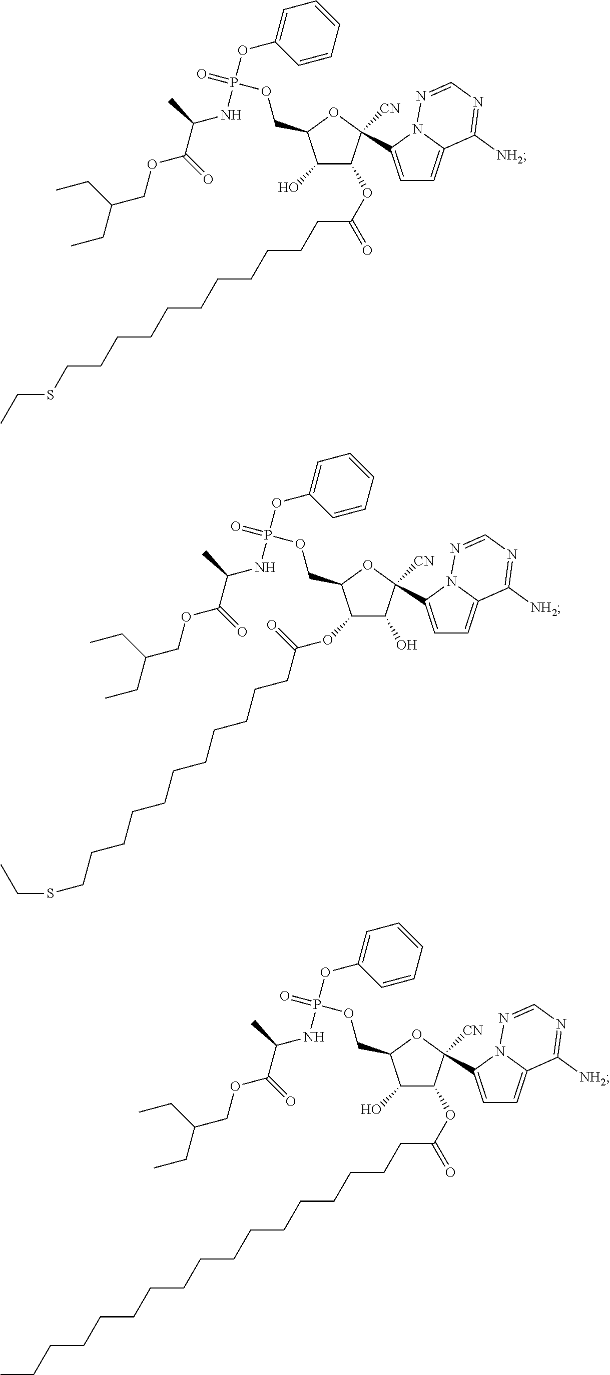 [SMILES: CCSCCCCCCCCCCCC(=O)O[C@@H]1[C@H](O)[C@@H](COP(=O)(N[C@H](C)C(=O)OCC(CC)CC)Oc2ccccc2)O[C@@]1(C#N)c1ccc2c(N)ncnn12 ; CCSCCCCCCCCCCCC(=O)O[C@H]1[C@@H](O)[C@](C#N)(c2ccc3c(N)ncnn23)O[C@@H]1COP(=O)(N[C@H](C)C(=O)OCC(CC)CC)Oc1ccccc1 ; CCCCCCCCCCCCCCCCCC(=O)O[C@@H]1[C@H](O)[C@@H](COP(=O)(N[C@H](C)C(=O)OCC(CC)CC)Oc2ccccc2)O[C@@]1(C#N)c1ccc2c(N)ncnn12] 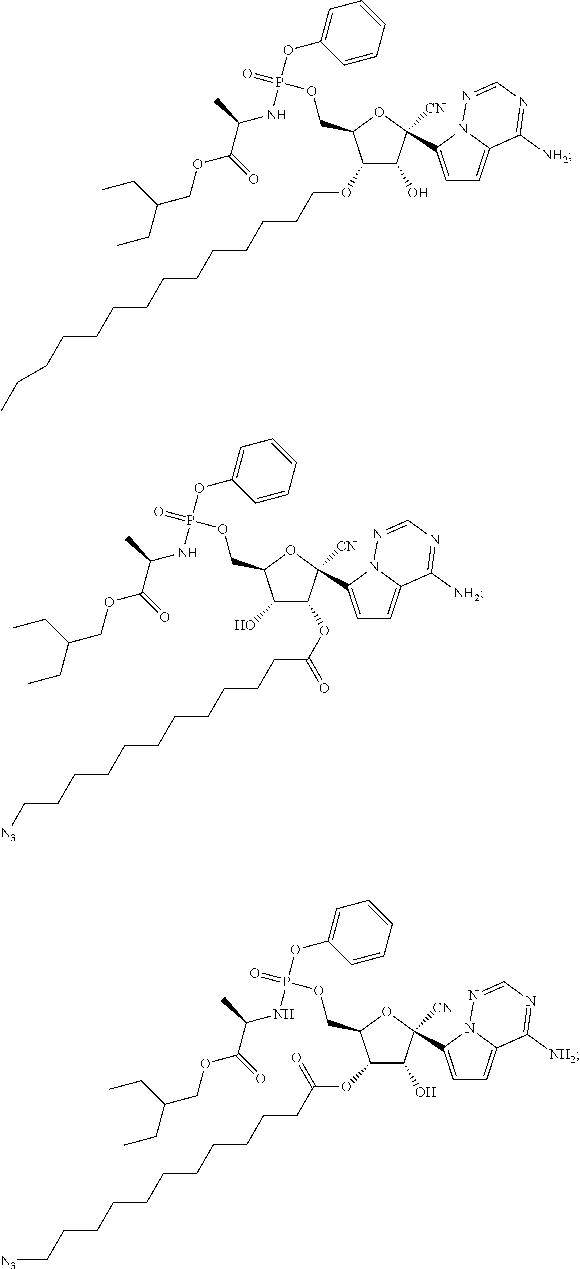 [SMILES: CCCCCCCCCCCCCCO[C@H]1[C@@H](O)[C@](C#N)(c2ccc3c(N)ncnn23)O[C@@H]1COP(=O)(N[C@H](C)C(=O)OCC(CC)CC)Oc1ccccc1 ; CCC(CC)COC(=O)[C@@H](C)NP(=O)(OC[C@H]1O[C@@](C#N)(c2ccc3c(N)ncnn23)[C@H](OC(=O)CCCCCCCCCCCN=[N+]=[N-])[C@@H]1O)Oc1ccccc1 ; CCC(CC)COC(=O)[C@@H](C)NP(=O)(OC[C@H]1O[C@@](C#N)(c2ccc3c(N)ncnn23)[C@H](O)[C@@H]1OC(=O)CCCCCCCCCCCN=[N+]=[N-])Oc1ccccc1] 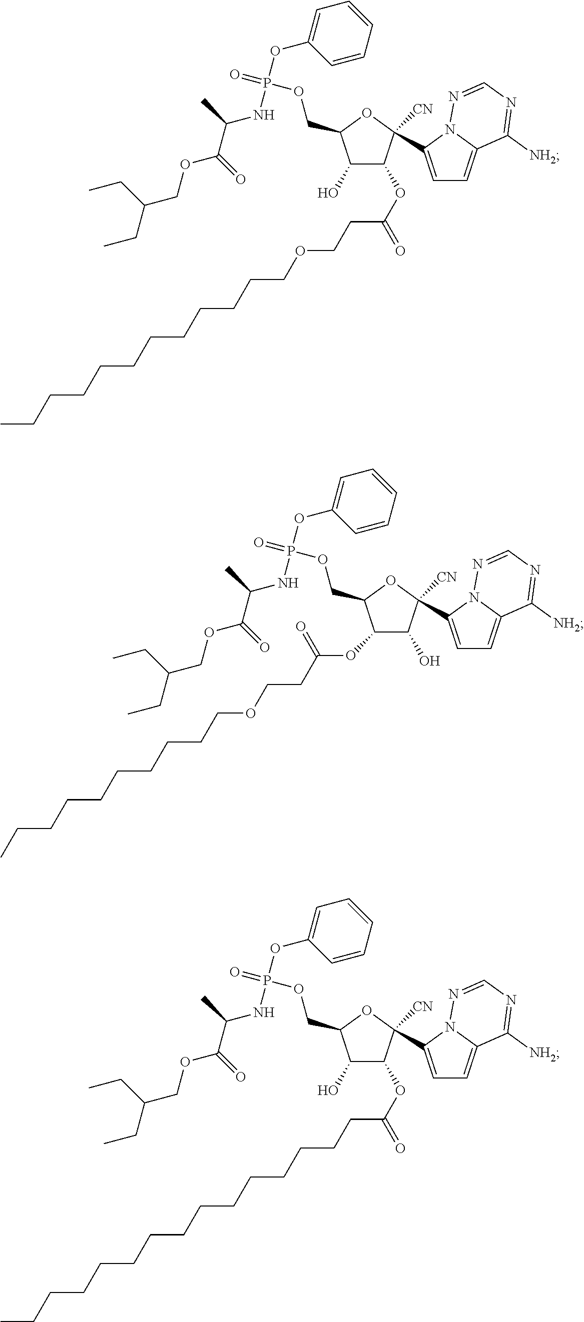 [SMILES: CCCCCCCCCCCCOCCC(=O)O[C@@H]1[C@H](O)[C@@H](COP(=O)(N[C@H](C)C(=O)OCC(CC)CC)Oc2ccccc2)O[C@@]1(C#N)c1ccc2c(N)ncnn12 ; CCCCCCCCCCOCCC(=O)O[C@H]1[C@@H](O)[C@](C#N)(c2ccc3c(N)ncnn23)O[C@@H]1COP(=O)(N[C@H](C)C(=O)OCC(CC)CC)Oc1ccccc1 ; CCCCCCCCCCCCCCCC(=O)O[C@@H]1[C@H](O)[C@@H](COP(=O)(N[C@H](C)C(=O)OCC(CC)CC)Oc2ccccc2)O[C@@]1(C#N)c1ccc2c(N)ncnn12] 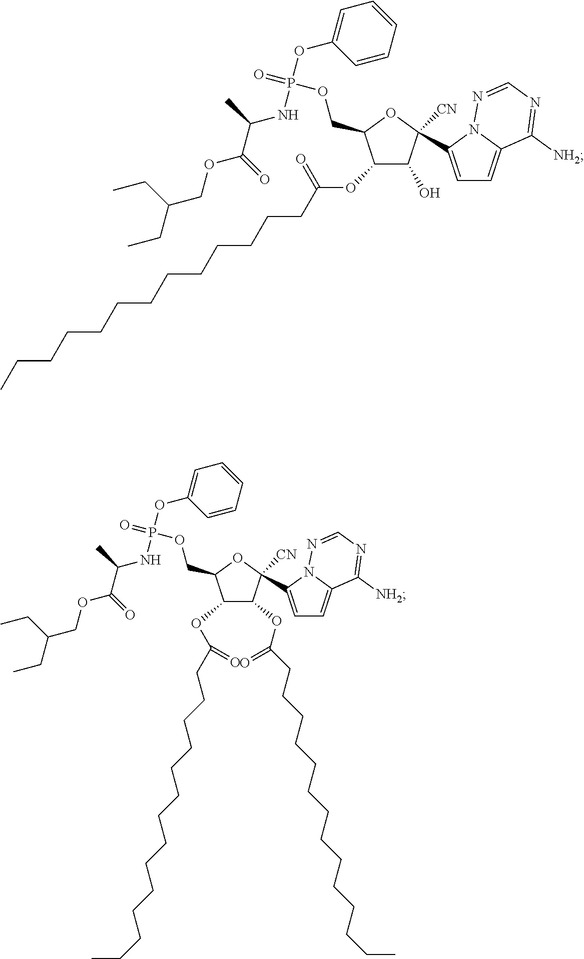 [SMILES: CCCCCCCCCCCCCC(=O)O[C@H]1[C@@H](O)[C@](C#N)(c2ccc3c(N)ncnn23)O[C@@H]1COP(=O)(N[C@H](C)C(=O)OCC(CC)CC)Oc1ccccc1 ; CCCCCCCCCCCCCCC(=O)O[C@H]1[C@@H](OC(=O)CCCCCCCCCCCCCC)[C@](C#N)(c2ccc3c(N)ncnn23)O[C@@H]1COP(=O)(N[C@H](C)C(=O)OCC(CC)CC)Oc1ccccc1] 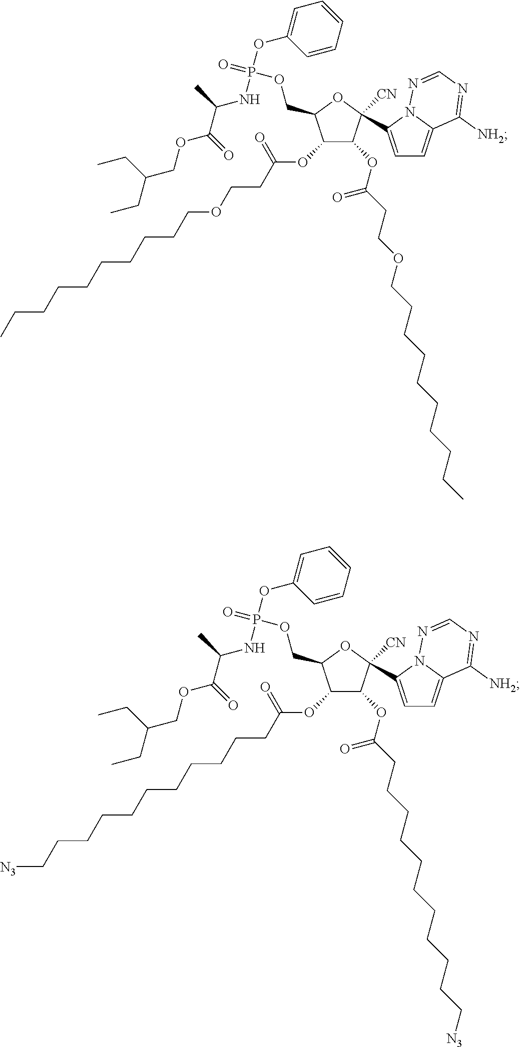 [SMILES: CCCCCCCCCCOCCC(=O)O[C@H]1[C@@H](OC(=O)CCOCCCCCCCCCC)[C@](C#N)(c2ccc3c(N)ncnn23)O[C@@H]1COP(=O)(N[C@H](C)C(=O)OCC(CC)CC)Oc1ccccc1 ; CCC(CC)COC(=O)[C@@H](C)NP(=O)(OC[C@H]1O[C@@](C#N)(c2ccc3c(N)ncnn23)[C@H](OC(=O)CCCCCCCCCCCN=[N+]=[N-])[C@@H]1OC(=O)CCCCCCCCCCCN=[N+]=[N-])Oc1ccccc1] 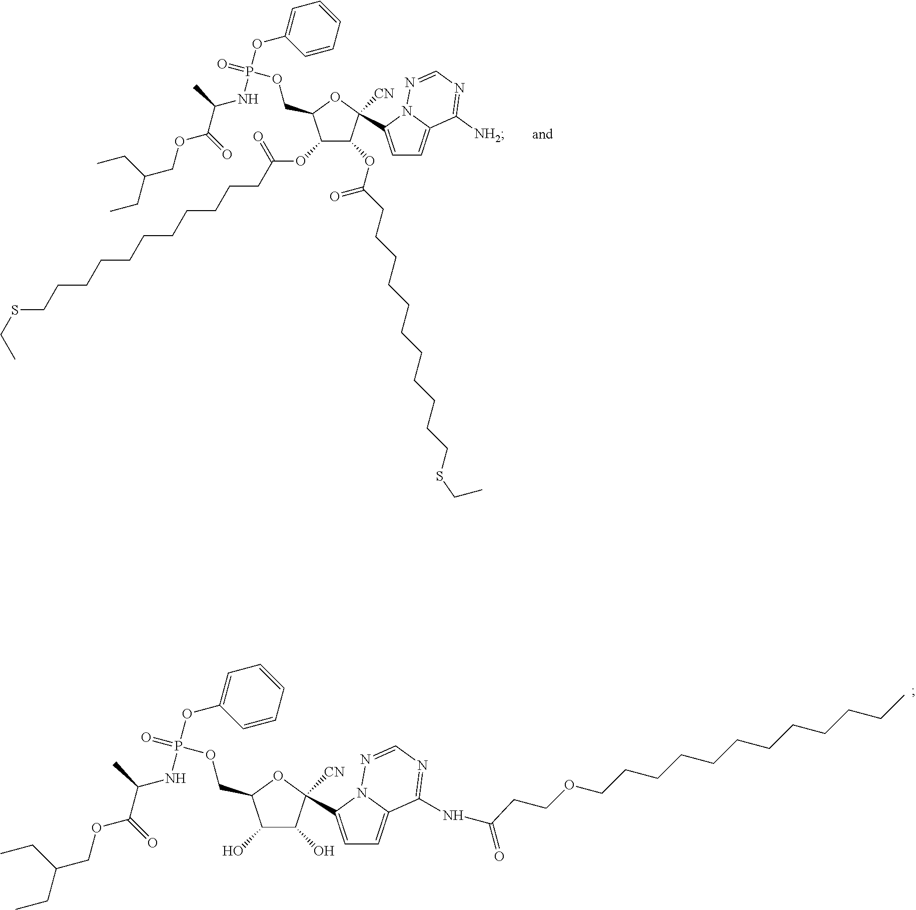 [SMILES: CCSCCCCCCCCCCCC(=O)O[C@H]1[C@@H](OC(=O)CCCCCCCCCCCSCC)[C@](C#N)(c2ccc3c(N)ncnn23)O[C@@H]1COP(=O)(N[C@H](C)C(=O)OCC(CC)CC)Oc1ccccc1 ; CCCCCCCCCCCCOCCC(=O)Nc1ncnn2c([C@]3(C#N)O[C@H](COP(=O)(N[C@H](C)C(=O)OCC(CC)CC)Oc4ccccc4)[C@@H](O)[C@H]3O)ccc12]
or a pharmaceutically acceptable salt thereof.

23-45. (canceled)

46. A pharmaceutical composition comprising a compound of claim 1, or a pharmaceutically acceptable salt thereof, and a pharmaceutically acceptable carrier.

47. The pharmaceutical composition of claim 46 formulated as an injectable, a solid or semi-solid form, a tablet, a film, a gel, a cream, an ointment, a spray, a solution, a suspension, a micellar suspension, a powder or granule, an encapsulated granule, an atomized mist, or a pessary.

48. The pharmaceutical composition of claim 46, further comprising a second active compound.

49. The pharmaceutical composition of claim 48, wherein the second active compound is selected from the group consisting of an antiviral compound, an antibacterial compound, an antifungal compound, an anti-inflammatory compound, and a bronchodilator.

50. (canceled)

51. A method of treating or preventing an infection with a virus in a subject comprising administering to the subject a therapeutically effective amount of a compound of claim 1.

52. The method of claim 51, wherein the virus is a coronavirus.

53. The method of claim 52, wherein the coronavirus is SARS-CoV, MERS-CoV, or SARS-CoV-2.

54-70. (canceled)
